(19) United States
(12) Patent Application Publication
(10) Pub. No.: US 2011/0319362 A1
(43) Pub. Date: Dec. 29, 2011

(54) STAT3 LIGANDS AND THERAPEUTIC USES THEREOF

(76) Inventors: Shaomeng Wang, Saline, MI (US); Jianyong Chen, Ann Arbor, MI (US); Cindy Gomez, Ann Arbor, MI (US); Longchuan Bai, Ann Arbor, MI (US); Zaneta Nikolovska-Coleska, Ann Arbor, MI (US); Yu-Jun Zhao, Jieshou (CN)

(21) Appl. No.: 13/128,941

(22) PCT Filed: Dec. 7, 2009
(86) PCT No.: PCT/US09/66932

§ 371 Date: Sep. 13, 2011

Related U.S. Application Data
(60) Provisional application No. 61/120,517, filed on Dec. 8, 2008.

(51) Int. Cl. A61K 31/675; C07D 487/04; A61P 35/00; A61K 31/407; A61K 31/4178; C07F 9/6561; C07D 495/04

(52) U.S. Cl. 514/80; 540/461; 514/413; 514/397

(57) ABSTRACT

Inhibitors of STAT3 are disclosed. Methods of using the STAT3 inhibitors in the treatment of diseases and conditions wherein inhibition of STAT3 provides a benefit, like cancers, also are disclosed.

### CROSS-REFERENCE TO RELATED APPLICATION

This application claims the benefit of U.S. Provisional patent application No. 61/120,517, filed Dec. 8, 2008, incorporated herein by reference in its entirety.

### FIELD OF THE INVENTION

The present invention relates to STAT3 inhibitors and to therapeutic methods of treating conditions and diseases wherein inhibition of STAT3 provides a benefit.

### BACKGROUND OF THE INVENTION

Signal Transducer and Activator of Transcription 3 (STAT3) belongs to the STAT family of proteins, which are both signal transducers and transcription factors. STAT proteins originally were discovered as latent cytoplasmic transcription factors that mediate cytokine and growth factor responses (J. E. Darnell, Jr., Recent Prog. Horm. Res. 51, 391-408 (1996); J. E. Darnell, Jr., Science 277, 1630-1635 (1997)). At least seven members in this family have been identified, namely, STAT1, STAT2, STAT3, STAT4, STAT5A, STAT5B, and STAT6, which are encoded by distinct genes.

The STAT family mediates several physiological effects, including growth and differentiation, survival, development, and inflammation. STAT3 has received particular attention among the seven STAT family members because it is hyperactivated in many human tumors.

In particular, STAT3 is well established as a critical molecule in biological processes leading to cancer development. Under normal biological conditions, STAT3 activation is rapid and transient. In contrast to normal STAT signaling, many human solid and hematological tumors harbor aberrant STAT3 activity. Activated STAT3 mediates critical gene expression changes and molecular events that dysregulate cell growth and apoptosis, promote angiogenesis, invasion, metastasis, and the development of resistance to apoptosis, and suppress the host's immune surveillance of the tumor, thereby making constitutively-active STAT3 a critical mediator of carcinogenesis and tumor progression.

Abnormal activation of STAT3 has been linked to a number of cancers. STAT3 is abnormally activated with high frequency in the carcinomas of the breast, head and neck squamous cell carcinoma, ovarian carcinoma, and skin melanomas. Abnormal STAT3 activation also correlates with the progression of diverse hematopoietic malignancies, such as various leukemias and lymphomas, and STAT3 is frequently activated in both multiple myeloma cell lines and tumor cell lines derived from patient bone marrows.

Initial work demonstrated that inhibition of persistently activated STAT3 specifically suppressed cancer cell survival and included tumor regression. Overall, studies have provided compelling evidence of the critical role of aberrant STAT3 in malignant transformation and tumorigenesis. Accordingly, inhibition of STAT3 signaling is an effective therapeutic approach to treat cancers.

STAT3 has received particular attention among the seven members of the seven members of the STAT family because it is considered a target for the treatment of human tumors. Inhibition of STAT3 signaling has increased the apoptotic rate of STAT3-dependent tumor cells. Because the function of the STAT3 SH2 domain is crucial for both STAT3 activation and nuclear translocation, STAT3 signaling can be inhibited by small molecules that impair the function of the STAT3 SH2 domain. Therefore, effective inhibition of aberrantly activated STAT3 represents an excellent target for anticancer drug design. While numerous compounds have been reported to inhibit STAT3 signaling, the vast majority act on targets other than STAT3.

Despite intense efforts, the design and discovery of truly effective STAT3 inhibitors remains a challenge. Accordingly, a need still exists in the art for potent STAT3 inhibitors having physical and pharmacological properties that permit use of the inhibitors in therapeutic applications. The present invention provides compounds designed to bind to STAT3 and inhibit STAT3 activity.

### SUMMARY OF THE INVENTION

The present invention is directed to inhibitors of STAT3 and to methods of using the inhibitors in a therapeutic treatment of conditions and diseases wherein inhibition of STAT3 activity provides a benefit. The present compounds are potent inhibitors of STAT3 activation and nuclear translocation, and induce apoptosis of STAT3-dependent cancer cell lines. The present compounds therefore are useful for the inhibition of STAT3 activation and activity, and for the disruption of aberrantly high STAT3 activity in cancer cell lines and tumor models.

More particularly, the present invention is directed to compounds having a structural formula (I):

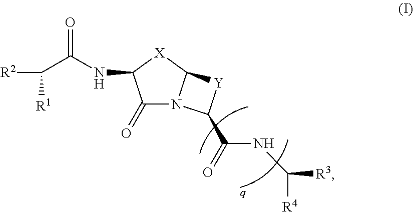

wherein X is (CH₂)ₙ and n is 1-6, wherein one CH₂ can be substituted by a heteroatom and CH₂ optionally can be substituted;

Y is (CH₂)ₘ and m is 1-3, wherein one CH₂ can be substituted by a heteroatom and CH₂ optionally can be substituted;

q is 0 or 1;

R¹ is

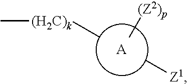

A is phenyl or a 5 or 6-membered heteroaryl ring, k is 0, 1, or 2, and p is 0 or 1, or R¹ is (CH₂)₁₋₆P(O)(OR^{a})₂;

Z¹, Z², independently, are OPO(OR^{a})₂, CH₂PO₃(R^{a})₂, OCH₂PO₃(H)(R^{a}), OCHFPO₃(R^{a})₂, (CH₂)₁₋₆CO₂R^{a}, (CH₂)₁₋₆P(O)(OH)(R^{a}), OCF₂PO₃(R^{a})₂, OCH(COOR^{a})₂, O(CH₂)₁₋₃CH(COOR^{a})₂, O(CH₂)₁₋₃COOR^{a}, O(CH₂)₁₋₃COR^{a}, OR^{a}, CON(R^{a})₂, or COOR^{a};

R² is H, NR^{a}R^{b}, NR^{a}C(═O)R^{b}, NR^{a}SOR^{b}, NR^{a}SO₂R^{b}, NR^{a}C(═O)OR^{b}, NR^{a}C(═O)NR^{b}R^{c}, NR^{a}C(═S)NR^{b}R^{c}, or NR^{a}C(═NH)NR^{b}R^{c};

or R² is null and R¹ is

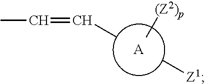

or R¹ and R² are taken together with the carbon atom to which they are attached to form a 5- to 10-membered monocyclic or bicyclic heteroaryl group substituted having a Z¹ group;

R³ is (CH₂)ⱼC(═O)NR^{a}R^{b}, (CH₂)ⱼNR^{a}C(═O)R^{b}, (CH₂)ⱼC(═O)R^{a}, (CH₂)ⱼNR^{a}(C═O)NR^{b}R^{c}, (CH₂)ⱼCH(OH)CH₂OR^{a}, C₁₋₆alkyl, NR^{a}C(═O)OR^{b}, NR^{a}R^{b}, (CH₂)ⱼNR^{a}(═NH)R^{b}R^{c}, C₁₋₆alkylNR^{a}R^{b}, (CH₂)ⱼNR^{a}C(═S)NR^{b}R^{c}, (CH₂)ⱼNR^{a}R^{b},R³ is (CH₂)ⱼC(═O)NR^{a}R^{b}, (CH₂)ⱼNR^{a}C(═O)R^{b}, (CH₂)ⱼC(═O)R^{a}, (CH₂)ⱼNR^{a}C(═O)NR^{b}R^{c}, (CH₂)ⱼCH(OH)CH₂OR^{a}, C₁₋₆alkyl, NR^{a}C(═O)OR^{b}, NR^{a}R^{b}, C₁₋₆alkylNR^{a}R^{b}, (CH₂)ⱼNR^{a}C(═S)NR^{b}R^{c}, (CH₂)ⱼNR^{a}R^{b},

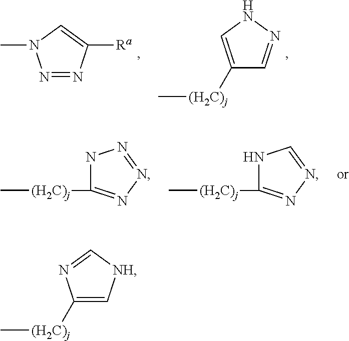

and j is 1, 2, 3, or 4; and

R⁴ is H, R^{a}, or CONR^{a}R^{b}; and

R^{a}, R^{b}, R^{c}, independently, is hydrogen, alkyl, substituted alkyl, aryl, substituted aryl, heteroaryl, substituted heteroaryl, C₃₋₈cycloalkyl, heterocycloalkyl, C_{1 -6}alkyleneheterocycloalkyl, substituted C₁₋₆alkyleneheterocycloalkyl, C₁₋₆alkylenearyl, substituted C₁₋₆alkylenearyl, C₁₋₆alkyleneheteroaryl, substituted C₁₋₆alkyleneheteroaryl, and (CH₂)₁₋₃(OCH₂)₁₋₃(OCH₂CH₂)₁₋₆NHR^{d}; and

R^{d} is hydrogen or

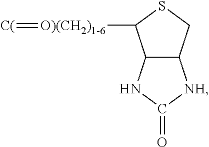

or a pharmaceutically acceptable salt, prodrug, hydrate, or solvate thereof.

In some preferred embodiments, the STAT3 inhibitor has a structural formula (II):

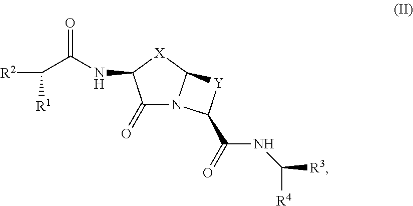

wherein X is (CH₂)ₙ and n is 1-6, wherein one CH₂ can be substituted by a O, S, or NR^{a}, and CH₂ optionally can be substituted;

Y is (CH₂)ₘ and m is 1-3, wherein one CH₂ can be substituted by a O, S, or NR^{a}, and CH₂ optionally can be substituted;

R¹ is

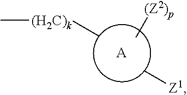

A is phenyl or a 5 or 6-membered heteroaryl ring, k is 0, 1, or 2, and p is 0 or 1, or R¹ is (CH₂)₁₋₆P(O)(OR^{a})₂;

Z¹, Z², independently, are OPO(OR^{a})₂, CH₂PO₃(R^{a})₂, OCH₂PO₃(H)(R^{a}), OCHFPO₃(R^{a})₂, (CH₂)₁₋₆CO₂R^{a}, (CH₂)₁₋₆P(O)(OH)(R^{a}), OCF₂PO₃(R^{a})₂, OCH(COOR^{a})₂, O(CH₂)₁₋₃CH(COOR^{a})₂, O(CH₂)₁₋₃COOR^{a}, O(CH₂)₁₋₃COR^{a}, OR^{a}, CON(R^{a})₂, or COOR^{a};

R² is H, NR^{a}R^{b}, NR^{a}C(═O)R^{b}, NR^{a}SOR^{b}, NR^{a}SO₂R^{b}, NR^{a}C(═O)OR^{b}, NR^{a}C(═O)NR^{b}R^{c}, NR^{a}C(═S)NR^{b}R^{c}, or NR^{a}C(═NH)NR^{b}R^{c};

or R² is null and R¹ is

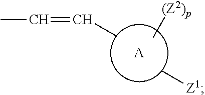

or R¹ and R² are taken together with the carbon atom to which they are attached to form a 5- to 10-membered monocyclic or bicyclic heteroaryl group substituted having a Z¹ group;

R³ is (CH₂)ⱼC(═O)NR^{a}R^{b}, (CH₂)ⱼNR^{a}C(═O)R^{b}, (CH₂)ⱼC(═O)R^{a}, (CH₂)ⱼNR^{a}(C═O)NR^{b}R^{c}, (CH₂)ⱼCH(OH)CH₂OR^{a}, C₁₋₆alkyl, NR^{a}C(═O)OR^{b}, NR^{a}R^{b}, (CH₂)ⱼNR^{a}(═NH)R^{b}R^{c}, C₁₋₆alkylNR^{a}R^{b}, (CH₂)ⱼNR^{a}C(═S)NR^{b}R^{c}, (CH₂)ⱼNR^{a}R^{b},R³ is (CH₂)ⱼC(═O)NR^{a}R^{b}, (CH₂)ⱼNR^{a}C(═O)R^{b}, (CH₂)ⱼC(═O)R^{a}, (CH₂)ⱼNR^{a}C(═O)NR^{b}R^{c}, (CH₂)ⱼCH(OH)CH₂OR^{a}, C₁₋₆alkyl, NR^{a}C(═O)OR^{b}, NR^{a}R^{b}, C₁₋₆alkylNR^{a}R^{b}, (CH₂)ⱼNR^{a}C(═S)NR^{b}R^{c}, (CH₂)ⱼNR^{a}R^{b},

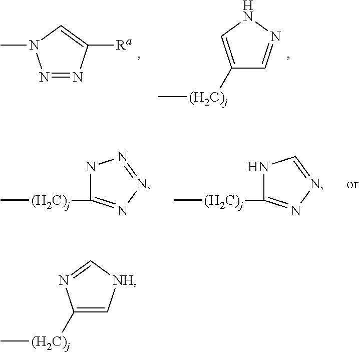

and j is 1, 3, or 4; and

R⁴ is H, R^{a}, or CONR^{a}R^{b}; and

R^{a}, R^{b}, R^{c}, independently, is hydrogen, alkyl, substituted alkyl, aryl, substituted aryl, heteroaryl, substituted heteroaryl, C₃₋₈cycloalkyl, heterocycloalkyl, C₁₋₆alkyleneheterocycloalkyl, substituted C₁₋₆alkyleneheterocycloalkyl, C₁₋₆alkylenearyl, substituted C₁₋₆alkylenearyl, C₁₋₆alkyleneheteroaryl, substituted C₁₋₆alkyleneheteroaryl, and (CH₂)₁₋₃(OCH₂)₁₋₃(OCH₂CH₂)₁₋₆NHR^{d}; and

R^{d} is hydrogen or

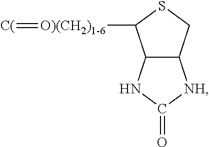

or a pharmaceutically acceptable salt, prodrug, hydrate, or solvate thereof.

In one embodiment, the present invention provides a method of treating a condition or disease by administering a therapeutically effective amount of a compound of structural formula (I) or (II) to an individual in need thereof. The disease or condition of interest is treatable by inhibition of STAT3, for example, a cancer.

Another embodiment of the present invention is to provide a composition comprising (a) a STAT3 inhibitor of structural formula (I) or (II) and (b) an excipient and/or pharmaceutically acceptable carrier useful in treating diseases or conditions wherein inhibition of STAT3 provides a benefit.

Another embodiment of the present invention is to utilize a composition comprising a compound of structural formula (I) or (II) and a second therapeutically active agent in a method of treating an individual for a disease or condition wherein inhibition of STAT3 provides a benefit.

In a further embodiment, the invention provides for use of a composition comprising a STAT3 inhibition of structural formula (I) or (II) and an optional second therapeutic agent for the manufacture of a medicament for treating a disease or condition of interest, e.g., a cancer.

Still another embodiment of the present invention is to provide a kit for human pharmaceutical use comprising (a) a container, (b1) a packaged composition comprising a STAT3 inhibitor of structural formula (I) or (II), and, optionally, (b2) a packaged composition comprising a second therapeutic agent useful in the treatment of a disease or condition of interest, and (c) a package insert containing directions for use of the composition or compositions, administered simultaneously or sequentially, in the treatment of the disease or condition.

The STAT3 inhibitor of structural formula (I) or (II) and the second therapeutic agent can be administered together as a single-unit dose or separately as multi-unit doses, wherein the STAT3 inhibitor of structural formula (I) or (II) is administered before the second therapeutic agent or vice versa. It is envisioned that one or more dose of a STAT3 inhibitor of structural formula (I) or (II) and/or one or more dose of a second therapeutic agent can be administered.

In one embodiment, a STAT3 inhibitor of structural formula (I) or (II) and a second therapeutic agent are administered simultaneously. In related embodiments, a STAT3 inhibitor of structural formula (I) or (II) and second therapeutic agent are administered from a single composition or from separate compositions. In a further embodiment, the STAT3 inhibitor of structural formula (I) or (II) and second therapeutic agent are administered sequentially. A STAT3 inhibitor of structural formula (I) or (II), as used in the present invention, can be administered in an amount of about 0.005 to about 500 milligrams per dose, about 0.05 to about 250 milligrams per dose, or about 0.5 to about 100 milligrams per dose.

These and other aspects and features of the present invention will become apparent from the following detailed description of the preferred embodiments.

### DETAILED DESCRIPTION OF THE PREFERRED EMBODIMENTS

The present invention is described in connection with preferred embodiments. However, it should be appreciated that the invention is not limited to the disclosed embodiments. It is understood that, given the description of the embodiments of the invention herein, various modifications can be made by a person skilled in the art. Such modifications are encompassed by the claims below.

As used herein, the terms “treat,” “treating,” “treatment,” and the like refer to eliminating, reducing, or ameliorating a disease or condition and/or symptoms associated therewith. Although not precluded, treating a disease or condition does not require that the disease, condition or symptoms associated therewith be completely eliminated. As used herein, the term “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound of the invention to an individual in need of such treatment.

Within the meaning of the invention, “treatment” also includes relapse prophylaxis or phase prophylaxis, as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy.

The term “therapeutically effective amount” or “effective dose” as used herein refers to an amount of the active ingredient(s) that is(are) sufficient, when administered by a method of the invention, to efficaciously deliver the active ingredient(s) for the treatment of condition or disease of interest to an individual in need thereof. In the case of a cancer or other proliferation disorder, the therapeutically effective amount of the agent may reduce (i.e., retard to some extent and preferably stop) unwanted cellular proliferation; reduce the number of cancer cells; reduce the tumor size; inhibit (i.e., retard to some extent and preferably stop) cancer cell infiltration into peripheral organs; inhibit (i.e., retard to some extent and preferably stop) tumor metastasis; inhibit, to some extent, tumor growth; reduce STAT3 signaling in the target cells; and/or relieve, to some extent, one or more of the symptoms associated with the cancer. To extent the administered compound or composition prevents growth and/or kills existing cancer cells, it may be cytostatic and/or cytotoxic.

The term “container” means any receptacle and closure therefor suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

“Concurrent administration,” “administered in combination,” “simultaneous administration” and similar phrases mean that two or more agents are administered concurrently to the subject being treated. By “concurrently,” it is meant that each agent is administered simultaneously or sequentially in any order at different points in time. However, if not administered simultaneously, they are, in one aspect, administered sufficiently closely in time so as to provide the desired treatment effect of the combination of agents. Suitable dosing intervals and dosing order of the agents will be readily apparent to those skilled in the art. It also is contemplated that two or more agents are administered from separate compositions, and in one aspect, one composition is administered prior to administration of the other composition. Prior administration refers to administration of the agents within one day (24 hours). It is further contemplated that one agent is administered subsequent to administration of the other agent. Subsequent administration is meant to describe administration from 30 minutes of the second agent up to one day (24 hours) after administration of the first agent. Within 24 hours may include administration after 30 minutes, 1, 2, 3, 4, 5, 6, 7, 8, 9, 10, 11, 12, 16, 20, or 24 hours.

The use of the terms “a”, “an”, “the”, and similar referents in the context of describing the invention (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the invention and is not a limitation on the scope of the invention unless otherwise claimed. No language in the specification should be construed as indicating any non-claimed element as essential to the practice of the invention.

The present invention is directed to STAT3 inhibitors having a structural formula (I):

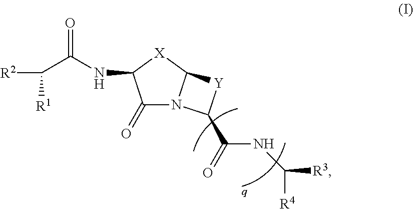

wherein X is (CH₂)ₙ and n is 1-6, wherein one CH₂ can be substituted by a heteroatom and CH₂ optionally can be substituted;

Y is (CH₂)ₘ and m is 1-3, wherein one CH₂ can be substituted by a heteroatom and CH₂ optionally can be substituted;

q is 0 or 1;

R¹ is

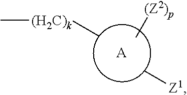

A is phenyl or a 5 or 6-membered heteroaryl ring, k is 0, 1, or 2, and p is 0 or 1, or R¹ is (CH₂)₁₋₆P(O)(OR^{a})₂;

Z¹, Z², independently, are OPO(OR^{a})₂, CH₂PO₃(R^{a})₂, OCH₂PO₃(H)(R^{a}), OCHFPO₃H₂, (CH₂)₁₋₆CO₂R^{a}, (CH₂)₁₋₆P(O)(OH)(R^{a}), OCF₂PO₃(R^{a})₂, OCH(COOR^{a})₂, O(CH₂)₁₋₃CH(COOR^{a})₂, O(CH₂)₁₋₃COOR^{a}, O(CH₂)₁₋₃COR^{a}, OR^{a}, CON(R^{a})₂, or COOR^{a};

R² is H, NR^{a}R^{b}, NR^{a}C(═O)R^{b}, NR^{a}SOR^{b}, NR^{a}SO₂R^{b}, NR^{a}C(═O)OR^{b}, NR^{a}C(═O)NR^{b}R^{c}, NR^{a}C(═S)NR^{b}R^{c}, or NR^{a}C(═NH)NR^{b}R^{c};

or R² is null and R¹ is

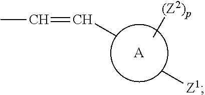

or R¹ and R² are taken together with the carbon atom to which they are attached to form a 5- to 10-membered monocyclic or bicyclic heteroaryl group substituted having a Z¹ group;

R³ is (CH₂)ⱼC(═O)NR^{a}R^{b}, (CH₂)ⱼNR^{a}C(═O)R^{b}, (CH₂)ⱼC(═O)R^{a}, (CH₂)ⱼNR^{a}(C═O)NR^{b}R^{c}, (CH₂)ⱼCH(OH)CH₂OR^{a}, C₁₋₆alkyl, NR^{a}C(═O)OR^{b}, NR^{a}R^{b}, (CH₂)ⱼNR^{a}(═NH)R^{b}R^{c}, C₁₋₆alkylNR^{a}R^{b}, (CH₂)ⱼNR^{a}C(═S)NR^{b}R^{c}, (CH₂)ⱼNR^{a}R^{b},R³ is (CH₂)ⱼC(═O)NR^{a}R^{b}, (CH₂)ⱼNR^{a}C(═O)R^{b}, (CH₂)ⱼC(═O)R^{a}, (CH₂)ⱼNR^{a}C(═O)NR^{b}R^{c}, (CH₂)ⱼCH(OH)CH₂OR^{a}, C₁₋₆alkyl, NR^{a}C(═O)OR^{b}, NR^{a}R^{b}, C₁₋₆alkylNR^{a}R^{b}, (CH₂)ⱼNR^{a}C(═S)NR^{b}R^{c}, (CH₂)ⱼNR^{a}R^{b},

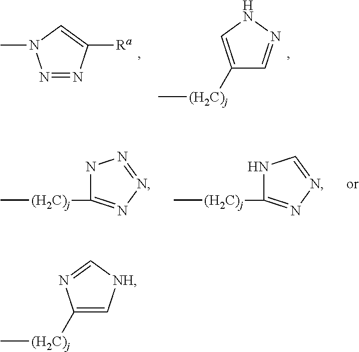

and j is 1, 2, 3, or 4; and

R⁴ is H, R^{a}, or CONR^{a}R^{b}; and

R^{a}, R^{b}, R^{c}, independently, is hydrogen, alkyl, substituted alkyl, aryl, substituted aryl, heteroaryl, substituted heteroaryl, C₃₋₈cycloalkyl, heterocycloalkyl, C₁₋₆alkyleneheterocycloalkyl, substituted C₁₋₆alkyleneheterocycloalkyl, C₁₋₆alkylenearyl, substituted C₁₋₆alkylenearyl, C₁₋₆alkyleneheteroaryl, substituted C₁₋₆alkyleneheteroaryl, and (CH₂)₁₋₃(OCH₂)₁₋₃(OCH₂CH₂)₁₋₆NHR^{d}; and

R^{d} is hydrogen or

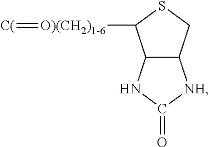

or a pharmaceutically acceptable salt, prodrug, hydrate, or solvate thereof.

In some preferred embodiments, the STAT3 inhibitor has a structural formula (II):

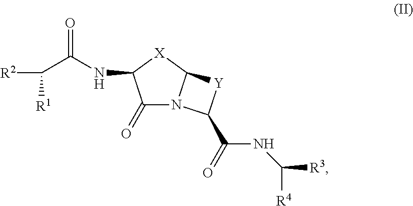

wherein X is (CH₂)ₙ and n is 1-6, wherein one CH₂ can be substituted by a O, S, or NR^{a}, and CH₂ optionally can be substituted;

Y is (CH₂)ₘ and m is 1-3, wherein one CH₂ can be substituted by a O, S, or NR^{a}, and CH₂ optionally can be substituted;

R¹ is

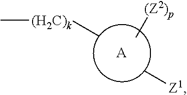

A is phenyl or a 5 or 6-membered heteroaryl ring, k is 0, 1, or 2, and p is 0 or 1, or R¹ is (CH₂)₁₋₆P(O)(OR^{a})₂;

Z¹, Z², independently, are OPO(OR^{a})₂, CH₂PO₃(R^{a})₂, OCH₂PO₃(H)(R^{a}), OCHFPO₃(R^{a})₂, (CH₂)₁₋₆CO₂R^{a}, (CH₂)₁₋₆P(O)(OH)(R^{a}), OCF₂PO₃(R^{a})₂, OCH(COOR^{a})₂, O(CH₂)₁₋₃CH(COOR^{a})₂, O(CH₂)₁₋₃COOR^{a}, O(CH₂)₁₋₃COR^{a}, OR^{a}, CON(R^{a})₂, or COOR^{a};

R² is H, NR^{a}R^{b}, NR^{a}C(═O)R^{b}, NR^{a}SOR^{b}, NR^{a}SO₂R^{b}, NR^{a}C(═O)OR^{b}, NR^{a}C(═O)NR^{b}R^{c}, NR^{a}C(═S)NR^{b}R^{c}, or NR^{a}C(═NH)NR^{b}R^{c}:

or R² is null and R¹ is

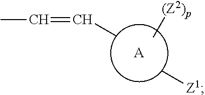

or R¹ and R² are taken together with the carbon atom to which they are attached to form a 5- to 10-membered monocyclic or bicyclic heteroaryl group substituted having a Z¹ group;

R³ is (CH₂)ⱼC(═O)NR^{a}R^{b}, (CH₂)ⱼNR^{a}C(═O)R^{b}, (CH₂)ⱼC(═O)R^{a}, (CH₂)ⱼNR^{a}(C═O)NR^{b}R^{c}, (CH₂)ⱼCH(OH)CH₂OR^{a}, C₁₋₆alkyl, NR^{a}C(═O)OR^{b}, NR^{a}R^{b}, (CH₂)ⱼNR^{a}(═NH)R^{b}R^{c}, C₁₋₆alkylNR^{a}R^{b}, (CH₂)ⱼNR^{a}C(═S)NR^{b}R^{c}, (CH₂)ⱼNR^{a}R^{b},R³ is (CH₂)ⱼC(═O)NR^{a}R^{b}, (CH₂)ⱼNR^{a}C(═O)R^{b}, (CH₂)ⱼC(═O)R^{a}, (CH₂)ⱼNR^{a}C(═O)NR^{b}R^{c}, (CH₂)ⱼCH(OH)CH₂OR^{a}, C₁₋₆alkyl, NR^{a}C(═O)OR^{b}, NR^{a}R^{b}, C₁₋₆alkylNR^{a}R^{b}, (CH₂)ⱼNR^{a}C(═S)NR^{b}R^{c}, (CH₂)ⱼNR^{a}R^{b},

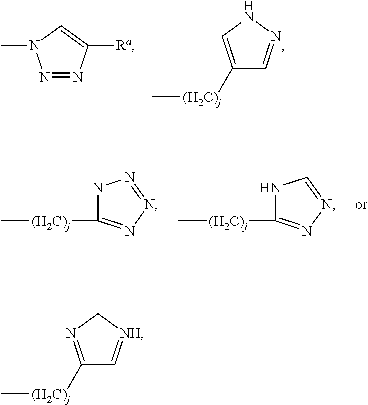

and j is 1, 2, 3, or 4; and

R⁴ is H, R^{a}, or CONR^{a}R^{b}; and

R^{a}, R^{b}, R^{c}, independently, is hydrogen, alkyl, substituted alkyl, aryl, substituted aryl, heteroaryl, substituted heteroaryl, C₃₋₈cycloalkyl, heterocycloalkyl, C₁₋₆alkyleneheterocycloalkyl, substituted C₁₋₆alkyleneheterocycloalkyl, C₁₋₆alkylenearyl, substituted C₁₋₆alkylenearyl, C₁₋₆alkyleneheteroaryl, substituted C₁₋₆alkyleneheteroaryl, and (CH₂)1-3(OCH₂)₁₋₃(OCH₂CH₂)₁₋₆NHR^{d}; and

R^{d} is hydrogen or

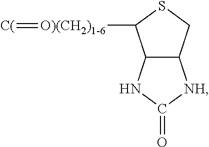

or a pharmaceutically acceptable salt, prodrug, hydrate, or solvate thereof.

The compounds of structural formula (I) and (II) inhibit STAT3 and are useful in the treatment of a variety of diseases and conditions. In particular, the compounds of structural formula (I) and (II) are used in methods of treating a disease or condition wherein inhibition of STAT3 provides a benefit, for example, cancers. The method comprises administering a therapeutically effective amount of a compound of structural formula (I) or (II) to an individual in need thereof. The present methods also encompass administering a second therapeutic agent to the individual in addition to the compound of structural formula (I) or (II). The second therapeutic agent is selected from drugs known as useful in treating the disease or condition afflicting the individual in need thereof, e.g., a chemotherapeutic agent and/or radiation known as useful in treating a particular cancer.

As used herein, the term “alkyl” refers to straight chained and branched saturated C₁₋₂₀ hydrocarbon groups, nonlimiting examples of which include methyl, ethyl, and straight chain and branched propyl, butyl, pentyl, hexyl, heptyl, and octyl, and C₁₀, C₁₂, C₁₄, C₁₆, C₁₈, and C₂₀ alkyl groups. The term Cₙ means the alkyl group has “n” carbon atoms. The term “alkylene” refers to an alkyl group having a substituent. An alkyl, e.g., methyl, or alkylene, e.g., —CH₂—, group can be substituted with halo, trifluoromethyl, trifluoromethoxy, hydroxy, alkoxy, nitro, cyano, alkylamino, or amino groups, for example.

As used herein, the term “halo” is defined as fluoro, chloro, bromo, and iodo.

The term “hydroxy” is defined as —OH.

The term “alkoxy” is defined as —OR, wherein R is alkyl.

The term “amino” is defined as —NH₂, and the term “alkylamino” is defined as —NR₂, wherein at least one R is alkyl and the second R is alkyl or hydrogen.

The term “nitro” is defined as —NO₂.

The term “cyano” is defined as —CN.

The term “trifluoromethyl” is defined as —CF₃.

The term “trifluoromethoxy” is defined as —OCF₃.

As used herein, groups such as

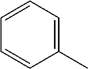

is an abbreviation for

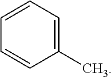

As used herein, groups such as C₁₋₃alkylphenyl means a C₁₋₃alkyl group bonded to a phenyl ring, for example,

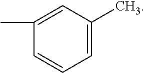

Groups such as C₁₋₃alkylenephenyl means a phenyl group bonded to a C₁₋₃alkylene group, for example

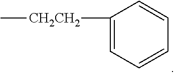

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four, groups independently selected from, for example, halo, alkyl, alkenyl, —OCF₃, —NO₂, —CN, —NC, —OH, alkoxy, amino, alkylamino, —CO₂H, —CO₂alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic or bicyclic ring system containing one or two aromatic rings and containing at least one nitrogen, oxygen, or sulfur atom in an aromatic ring. Unless otherwise indicated, a heteroaryl group can be unsubstituted or substituted with one or more, and in particular one to four, substituents selected from, for example, halo, alkyl, alkenyl, —OCF₃, —NO₂, —CN, —NC, —OH, alkoxy, amino, alkylamino, —CO₂H, —CO₂alkyl, aryl, and heteroaryl. Examples of heteroaryl groups include, but are not limited to, thienyl, furyl, oxazolyl, quinolyl, thiophenyl, isoquinolyl, indolyl, triazinyl, triazolyl, isothiazolyl, isoxazolyl, imidazolyl, benzothiazolyl, pyrimidinyl, thiazolyl, thiadiazolyl, pyridinyl, pyridazinyl, pyrazolyl, pyrazinyl, quinolyl, tetrazolyl, oxazolyl, pyrrolyl, benzimidazolyl, benzofuranyl, cinnolinyl, indazolyl, indolizinyl, phthalazinyl, triazinyl, isoindolyl, purinyl, oxadiazolyl, furazanyl, benzofurazanyl, benzothiophenyl, benzotriazolyl, benzothiazolyl, benzisothiazolyl, benzoxazolyl, benzisoxazolyl, benzimidazolyl, quinazolinyl, quinoxalinyl, napththyridinyl, dihydroquinolyl, tetrahydroquinolyl, dihydroisoquinolyl, tetrahydroisoquinolyl, benzofuryl, furopyridinyl, pyrrolopyrimidinyl, and azaindolyl.

Additional aryl and heteroaryl groups are disclosed in Appendix A, which constitutes a portion of the present disclosure.

As used herein, the term “C₃₋₈cycloalkyl” means a monocyclic aliphatic ring containing three to eight carbon atoms.

As used herein, the term “heterocycloalkyl” means a monocyclic or a bicyclic aliphatic ring containing 5 to 10 total atoms, of which one to five of the atoms are independently selected from nitrogen, oxygen, and sulfur and the remaining atoms are carbon.

In accordance with the present invention, X is (CH₂)ₙ, wherein n is 1-6. In preferred embodiments, n is 1-4. In some embodiments, one CH₂ group can be replaced by a heteroatom selected from O, S, and Me. In this embodiment, R^{a} typically is hydrogen or alkyl. One or more CH₂ group also can be substituted, independently, with halo, CF₃, OCF₃, OH, alkoxy, NO₂, CN, alkylamino, or amino.

Further, Y is (CH₂)ₘ, wherein m is 1-3. In preferred embodiments, m is 2 or 3. Like the X moiety, one CH₂ group of the Y moiety can be replaced by a heteroatom selected from O, S, and NR^{a}. In this embodiment, R^{a} typically is hydrogen or alkyl. One or more CH₂ group also can be substituted, independently, with halo, CF₃, OCF₃, OH, alkoxy, NO₂, CN, alkylamino, or amino.

In some preferred embodiments of the invention, q is 1.

The “A” ring of the R¹ group can be, for example,

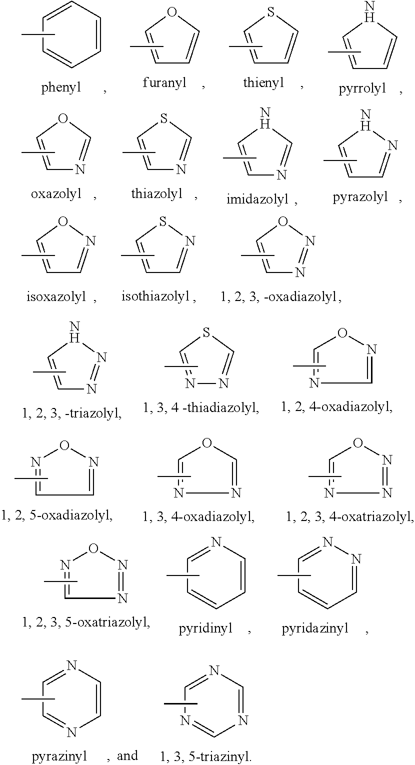

One preferred “A” ring is phenyl.

In some preferred embodiments of R¹, k is 1 or 2 or p is 0 or 1. In various embodiments, Z¹ is OPO₃(R^{a})₂, OCH(CO₂R^{a})₂, (CH₂)₂CO₂R^{a}, OR^{a}, OCH₂CO₂R^{a}, or (CH₂)₁₋₄PO₃(R^{a})₂. Specific Z¹ groups include, but are not limited to, OPO₃H₂, OCH(CO₂H)₂, (CH₂)₂CO₂(tBu), (CH₂)₂CO₂H, OH, OCH₂CO₂C₂H₅, OCH(CO₂C₂H₅)₂, OCH₂CO₂H, OPO(OCH₃)₂, CH₃PO₃H₂, CH₂P(O)(OH)(CH₃), (CH₂)₄P(O)(OH)(CH₃), OCH₂PO₃H₂, and OCH₂PO₃(H)(C₄H₁₀).

In various embodiments, Z² is CO₂R^{a}, for example, CO₂H.

In preferred embodiments, R² is H, N(R^{a})₂, or NR^{a}C(═O)R^{b}. Specific R² groups include, but are not limited to, H, NH₂, NHC(═O)CH₃, N(CH₃)₂, NHCH₃,

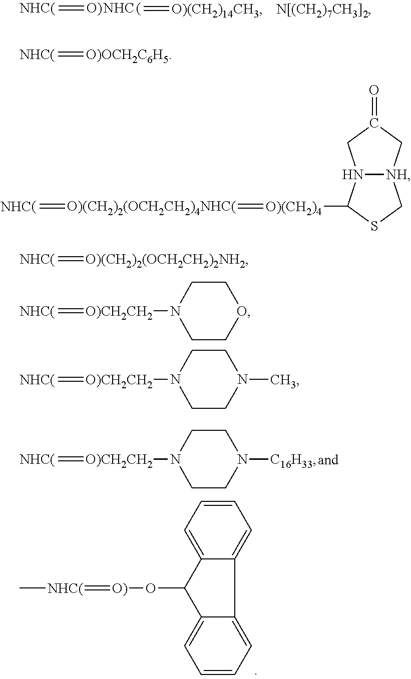

In preferred embodiments, R³ is (CH₂)ⱼC(═O)NR^{a}R^{b}, C₁₋₆alkyl, NR^{a}R^{b}, (CH₂)ⱼCH(OH)CH₂OR^{a}, NR^{a}C(═O)OR^{b},

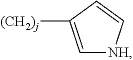

(CH₂)ⱼNR^{a}R^{b}, (CH₂)ⱼNR^{a}C(═O)R^{b}, (CH₂)ⱼNR^{a}(═NH)NR^{b}R^{c}, and

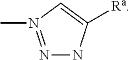

Specific R³ groups include, but are not limited to, (CH₂)₂C(═O)NH₂, CH₂CH(OH)CH₂OH, CH₃, CH₃CH₂, NH₂, NHC(═O)OCH₂C₆H₅, (CH₂)₃NH₂, (CH₂)₃N(CH₃)₂, (CH₂)₃NHC(═O)CH₃, (CH₂)₁₋₃NH(═NH)NH, (CH₂)₃NH₂, (CH₂)₂C(═O)NH(CH₃), (CH₂)₂C(═O)N(CH₃)₂,

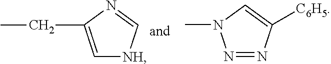

In preferred embodiments, R⁴ is H or C(═O)NR^{a}R^{b}. Specific R⁴ groups include, but are not limited to, H, C(═O)NHCH₂C₆H₅, C(═O)NHCH₂CH₂C₆H₅, C(═O)NH(CH₂)₄C₆H₅, C(═O)NH(CH₂)₆C₆H₅, and C(═O)NHCH₃.

Nonlimiting examples of heteroaryl groups wherein R¹ and R² are taken together with the carbon to which the attached include

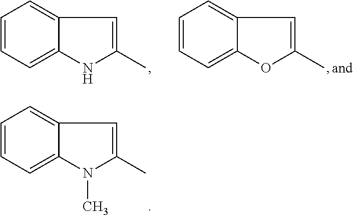

In other preferred embodiments, a compound of the present invention has a structure:

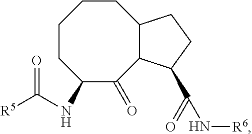

wherein
R⁵ is

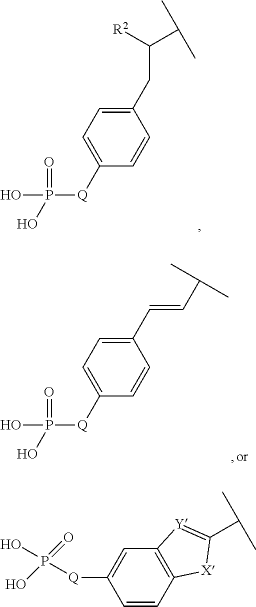

Q is O, CR₂, OCH₂, CF₂, CFH;
X′ is O, NH;
Y′ is CH, N, O; and
R⁶ is

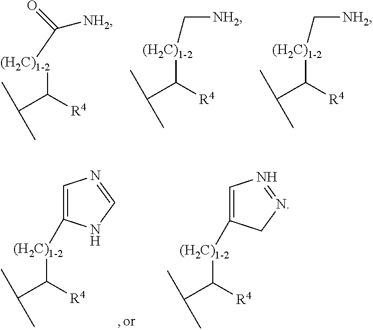

Additionally, salts, prodrugs, hydrates, and solvates of the present compounds also are included in the present invention and can be used in the methods disclosed herein. The present invention further includes all possible stereoisomers and geometric isomers of the compounds of structural formula (I) and (II). The present invention includes both racemic compounds and optically active isomers. When a compound of structural formula (I) or (II) is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-808 (1997). Resolution of the final product, an intermediate, or a starting material can be achieved by any suitable method known in the art. Additionally, in situations where tautomers of the compounds of structural formula (I) or (II) are possible, the present invention is intended to include all tautomeric forms of the compounds.

Prodrugs of compounds of structural formula (I) and (II) are included in the present invention. It is well established that a prodrug approach, wherein a compound is derivatized into a form suitable for formulation and/or administration, then released as a drug in vivo, has been successfully employed to transiently (e.g., bioreversibly) alter the physicochemical properties of the compound (see, H. Bundgaard, Ed., “Design of Prodrugs,” Elsevier, Amsterdam, (1985); R. B. Silverman, “The Organic Chemistry of Drug Design and Drug Action,” Academic Press, San Diego, chapter 8, (1992); K. M. Hillgren et al., Med. Res. Rev., 15, 83 (1995))

Compounds of the present invention can contain one or more functional groups. The functional groups, if desired or necessary, can be modified to provide a prodrug. Suitable prodrugs include, for example, acid derivatives, such as amides and esters. It also is appreciated by those skilled in the art that N-oxides can be used as a prodrug.

The following are examples of prodrugs for the compound of Example 36:

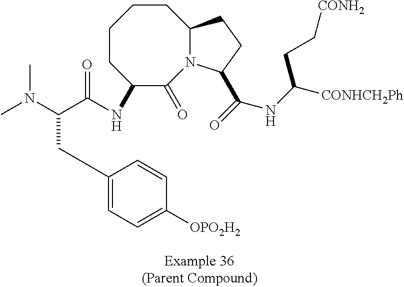

### EXAMPLES OF PRODRUGS

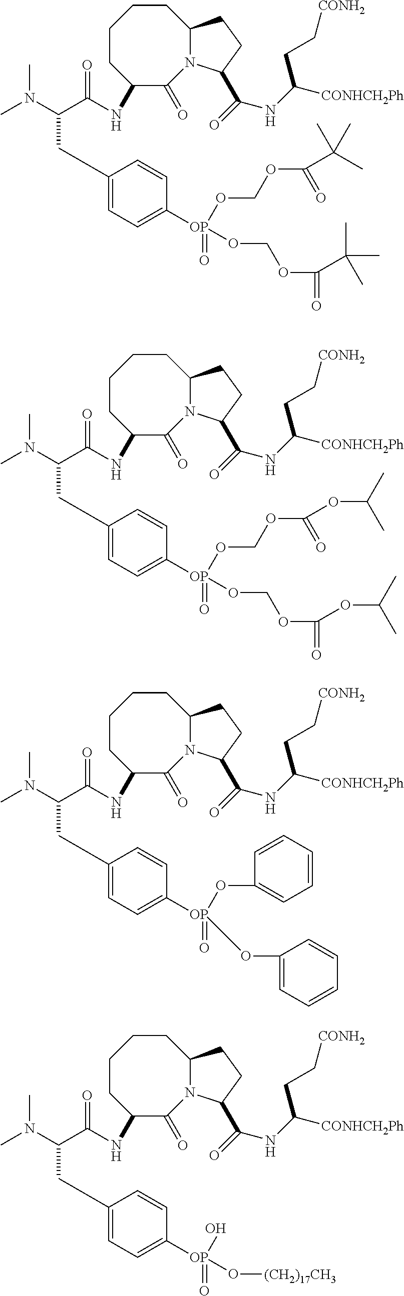

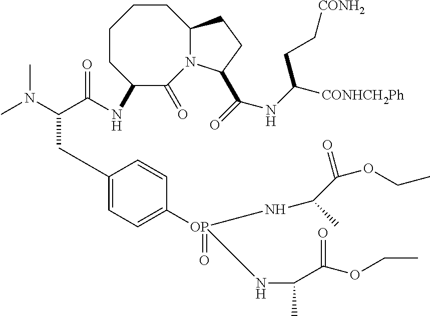

### Additional Prodrugs are Illustrated Below in Table 2.

Compounds of the invention can exist as salts. Pharmaceutically acceptable salts of the compounds of the invention often are preferred in the methods of the invention. As used herein, the term “pharmaceutically acceptable salts” refers to salts or zwitterionic forms of the compounds of structural formula (I) and (II). Salts of compounds of formula (I) and (II) can be prepared during the final isolation and purification of the compounds or separately by reacting the compound with an acid having a suitable cation. The pharmaceutically acceptable salts of compounds of structural formula (I) and (II) can be acid addition salts formed with phamiaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the invention include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to compounds of the present invention appearing herein is intended to include compounds of structural formula (I) and (II) as well as pharmaceutically acceptable salts, hydrates, solvates, or prodrugs thereof.

The compounds of structural formula (I) and (II) also can be conjugated or linked to auxiliary moieties that promote a beneficial property of the compound in a method of therapeutic use. Such conjugates can enhance delivery of the compounds to a particular anatomical site or region of interest (e.g., a tumor), enable sustained therapeutic concentrations of the compounds in target cells, alter pharmacokinetic and pharmacodynamic properties of the compounds, and/or improve the therapeutic index or safety profile of the compounds. Suitable auxiliary moieties include, for example, amino acids, oligopeptides, or polypeptides, e.g., antibodies, such as monoclonal antibodies and other engineered antibodies; and natural or synthetic ligands to receptors in target cells or tissues. Other suitable auxiliaries include fatty acid or lipid moieties that promote biodistribution and/or uptake of the compound by target cells (see, e.g., Bradley et al., Clin. Cancer Res. (2001) 7:3229).

Specific compounds of the present invention include, but are not limited to, compounds having the structure set forth below in Tables 1 and 2.

In some embodiments, a compound of structural formula (I) or (II) is a selective STAT3 inhibition which, because of a low affinity for other members of the STAT family, e.g., STAT1 and STAT5, give rise to fewer side effects than compounds that are non-selective STAT ligands.

The present invention provides STAT3 inhibitors, as exemplified by compounds of structural formula (I) and (II), for the treatment of a variety of diseases and conditions wherein inhibition of STAT3 has a beneficial effect. Preferably, a compound of structural formula (I) or (II) is selective for STAT3 over the other STAT family members by a factor of at least 100, and more preferably by a factor of at least 1000.

In one embodiment, the present invention relates to a method of treating an individual suffering from a disease or condition wherein inhibition of the STAT3 provides a benefit comprising administering a therapeutically effective amount of a compound of structural formula (I) or (II) to an individual in need thereof.

In some embodiments, a compound of structural formula (I) or (II) may be useful in the treatment of diseases and conditions wherein activation of STAT3 provides a benefit, such as immune response diseases and hypoxic or ischemic conditions or disorders.

The methods described herein relate to the use of a compound of structural formula (I) or (II) and an optional second therapeutic agent useful in the treatment of diseases and conditions wherein inhibition of STAT3 provides a benefit. The method of the present invention can be accomplished by administering a compound of structural formula (I) or (II) as the neat compound or as a pharmaceutical composition. Administration of a phaimaceutical composition, or neat compound of structural formula (I) or (II), can be performed during or after the onset of the disease or condition of interest. Typically, the pharmaceutical compositions are sterile, and contain no toxic, carcinogenic, or mutagenic compounds that would cause an adverse reaction when administered.

In many embodiments, a compound of structural formula (I) or (II) is administered in conjunction with a second therapeutic agent useful in the treatment of a disease or condition wherein inhibition of STAT3 provides a benefit. The second therapeutic agent is different from the compound of structural formula (I) or (II). A compound of structural formula (I) or (II) and the second therapeutic agent can be administered simultaneously or sequentially. In addition, the compound of structural formula (I) or (II) and second therapeutic agent can be administered from a single composition or two separate compositions. A compound of structural formula (I) or (II) and the optional second therapeutic agent can be administered simultaneously or sequentially to achieve the desired effect.

The second therapeutic agent is administered in an amount to provide its desired therapeutic effect. The effective dosage range for each second therapeutic agent is known in the art, and the second therapeutic agent is administered to an individual in need thereof within such established ranges.

The present invention therefore is directed to compositions and methods of treating diseases or conditions wherein inhibition of STAT3 provides a benefit. The present invention also is directed to pharmaceutical compositions comprising a compound of structural formula (I) or (II) and a second therapeutic agent useful in the treatment of diseases and conditions wherein inhibition of STAT3 provides a benefit. Further provided are kits comprising a compound of structural formula (I) or (II) and, optionally, a second therapeutic agent useful in the treatment of diseases and conditions wherein inhibition of STAT3 provides a benefit, packaged separately or together, and an insert having instructions for using these active agents.

A compound of structural formula (I) or (II) and the second therapeutic agent can be administered together as a single-unit dose or separately as multi-unit doses, wherein the compound of structural formula (I) or (II) is administered before the second therapeutic agent or vice versa. One or more dose of the compound of structural formula (I) or (II) and/or one or more dose of the second therapeutic agent can be administered. The compounds of structural formula (I) or (II) therefore can be used in conjunction with one or more second therapeutic agents, for example, but not limited to, anticancer agents.

Within the meaning of the present invention, the term “disease” or “condition” denotes disturbances and/or anomalies that as a rule are regarded as being pathological conditions or functions, and that can manifest themselves in the form of particular signs, symptoms, and/or malfunctions. As demonstrated below, a compound of structural formula (I) or (II) is a potent inhibition of STAT3 and can be used in treating diseases and conditions wherein inhibition of STAT3 provides a benefit.

The diseases and conditions that can be treated in accordance to the invention include, for example, cancers. A variety of cancers can be treated including, but not limited to: carcinomas, including bladder (including accelerated and metastic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, renal, and skin (including squamous cell carcinoma); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma, hematopoietic tumors of myeloid lineage, including acute and chronic myelogenous leukemias, myelodysplastic syndrome, myeloid leukemia, and promyelocytic leukemia; tumors of the central and peripheral nervous system, including astrocytoma, neuroblastoma, glioma, and schwannomas; tumors of mesenchymal origin, including fibrosarcoma, rhabdomyoscarcoma, and osteosarcoma; and other tumors, including melanoma, xenoderma pigmentosum, keratoactanthoma, seminoma, thyroid follicular cancer, teratocarcinoma, renal cell carcinoma (RCC), pancreatic cancer, myeloma, myeloid and lymphoblastic leukemia, neuroblastoma, and glioblastoma.

Additional forms of cancer treatable by the STAT3 inhibitors of the present invention include, for example, adult and pediatric oncology, growth of solid tumors/malignancies, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer (including small cell carcinoma, cutaneous T cell lymphoma, Hodgkin's lymphoma, non-Hodgkin's lymphoma, cancer of the adrenal cortex, ACTH-producing tumors, nonsmall cell cancers, breast cancer, including small cell carcinoma and ductal carcinoma), gastrointestinal cancers (including stomach cancer, colon cancer, colorectal cancer, and polyps associated with colorectal neoplasia), pancreatic cancer, liver cancer, urological cancers (including bladder cancer, such as primary superficial bladder tumors, invasive transitional cell carcinoma of the bladder, and muscle-invasive bladder cancer), prostate cancer, malignancies of the female genital tract (including ovarian carcinoma, primary peritoneal epithelial neoplasms, cervical carcinoma, uterine endometrial cancers, vaginal cancer, cancer of the vulva, uterine cancer and solid tumors in the ovarian follicle), malignancies of the male genital tract (including testicular cancer and penile cancer), kidney cancer (including renal cell carcinoma, brain cancer (including intrinsic brain tumors, neuroblastoma, astrocytic brain tumors, gliomas, and metastatic tumor cell invasion in the central nervous system), bone cancers (including osteomas and osteosarcomas), skin cancers (including malignant melanoma, tumor progression of human skin keratinocytes, and squamous cell cancer), thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma. Accordingly, administration of a present STAT3 inhibitor is expected to enhance treatment regimens.

It is theorized, but not relied upon, that the present compounds target the SH2 domain to serve as inhibitors of STAT3 function by (1) preventing docking to cell surface receptors or adapter molecules, thus preventing phosphorylation of Tyr 705, subsequent dimerization, nuclear translocation, and gene expression and/or (2) by breaking up dimers of prephosphorylated protein thus preventing translocation to the nucleus and DNA binding, which inhibits expression of downstream genes involved in survival, cell cycling, or angiogenesis. See J. S. McMurray, “Structural Basis for the Binding of High Affinity Phosphopeptides to Stat3,” Biopolymer, Volume 90, pages 69-79 (2007).

In the present method, a therapeutically effective amount of one or more compound (I) or (II), typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A compound of structural formula (I) or (II) can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a compound of structural formula (I) or (II) is administered in an effective amount to achieve its intended purpose. The exact formulation, route of administration, and dosage is determined by an individual physician in view of the diagnosed condition or disease. Dosage amount and interval can be adjusted individually to provide levels of a compound of structural formula (I) or (II) that is sufficient to maintain therapeutic effects.

Toxicity and therapeutic efficacy of the compounds of structural formula (I) and (II) can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. Compounds that exhibit high therapeutic indices are preferred. The data obtained from such data can be used in formulating a dosage range for use in humans. The dosage preferably lies within a range of circulating compound concentrations that include the ED₅₀ with little or no toxicity. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a compound of structural formula (I) or (II) required for use in therapy varies with the nature of the condition being treated, the length of time that activity is desired, and the age and the condition of the patient, and ultimately is determined by the attendant physician. Dosage amounts and intervals can be adjusted individually to provide plasma levels of the STAT3 inhibitor that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a present STAT3 inhibitor can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

The dosage of a composition containing a STAT3 inhibitor of structural formula (I) or (II), or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, 10 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. The above dosages are exemplary of the average case, but there can be individual instances in which higher or lower dosages are merited, and such are within the scope of this invention. In practice, the physician determines the actual dosing regimen that is most suitable for an individual patient, which can vary with the age, weight, and response of the particular patient.

A compound of structural formula (I) or (II) used in a method of the present invention can be administered in an amount of about 0.005 to about 500 milligrams per dose, about 0.05 to about 250 milligrams per dose, or about 0.5 to about 100 milligrams per dose. For example, a compound of structural formula (I) or (II) can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

In the treatment of a cancer, a compound of structural formula (1) or (II) can be administered with a chemotherapeutic agent and/or radiation.

Embodiments of the present invention employ electromagnetic radiation of: gamma-radiation (10⁻²⁰ to 10⁻¹³ m), X-ray radiation (10⁻¹² to 10⁻⁹ m), ultraviolet light (10 nm to 400 nm), visible light (400 nm to 700 nm), infrared radiation (700 nm to 1 mm), and microwave radiation (1 mm to 30 cm).

Many cancer treatment protocols currently employ radiosensitizers activated by electromagnetic radiation, e.g., X-rays. Examples of X-ray-activated radiosensitizers include, but are not limited to, metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR), 5-iododeoxyuridine (IUdR), bromodeoxycytidine, fluorodeoxyuridine (FUdR), hydroxyurea, cis-platin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, PHOTOFRIN®, benzoporphyrin derivatives, NPe6, tin etioporphyrin (SnET2), pheoborbide-a, bacteriochlorophyll-a, naphthalocyanines, phthalocyanines, zinc phthalocyanine, and therapeutically effective analogs and derivatives of the same.

Radiosensitizers can be administered in conjunction with a therapeutically effective amount of one or more compounds in addition to a present STATS inhibitor, such compounds including, but not limited to, compounds that promote the incorporation of radiosensitizers to the target cells, compounds that control the flow of therapeutics, nutrients, and/or oxygen to the target cells, chemotherapeutic agents that act on the tumor with or without additional radiation, or other therapeutically effective compounds for treating cancer or other disease. Examples of additional therapeutic agents that can be used in conjunction with radiosensitizers include, but are not limited to, 5-fluorouracil (5-FU), leucovorin, oxygen, carbogen, red cell transfusions, perfluorocarbons (e.g., FLUOSOLW®-DA), 2,3-DPG, BW12C, calcium channel blockers, pentoxifylline, antiangiogenesis compounds, hydralazine, and L-BSO.

The chemotherapeutic agent can be any pharmacological agent or compound that induces apoptosis. The pharmacological agent or compound can be, for example, a small organic molecule, peptide, polypeptide, nucleic acid, or antibody. Chemotherapeutic agents that can be used include, but are not limited to, alkylating agents, antimetabolites, hormones and antagonists thereof, natural products and their derivatives, radioisotopes, antibodies, as well as natural products, and combinations thereof For example, a STAT3 inhibitor of the present invention can be administered with antibiotics, such as doxorubicin and other anthracycline analogs, nitrogen mustards, such as cyclophosphamide, pyrimidine analogs such as 5-fluorouracil, cis-platin, hydroxyurea, taxol and its natural and synthetic derivatives, and the like. As another example, in the case of mixed tumors, such as adenocarcinoma of the breast, where the tumors include gonadotropin-dependent and gonadotropin-independent cells, the compound can be administered in conjunction with leuprolide or goserelin (synthetic peptide analogs of LH-RH). Other antineoplastic protocols include the use of an inhibitor compound with another treatment modality, e.g., surgery or radiation, also referred to herein as “adjunct anti-neoplastic modalities.” Additional chemotherapeutic agents useful in the invention include hormones and antagonists thereof, radioisotopes, antibodies, natural products, and combinations thereof.

Examples of chemotherapeutic agents useful in a method of the present invention are listed in the following table.

TABLE 1

Alkylating agents
Nitrogen mustards
mechlorethamine
cyclophosphamide
ifosfamide
melphalan
chlorambucil
uracil mustard
temozolomide
Nitrosoureas
carmustine (BCNU)
lomustine (CCNU)
semustine (methyl-CCNU)
chlormethine
streptozocin
Ethylenimine/Methyl-melamine
triethylenemelamine (TEM)
triethylene thiophosphoramide
(thiotepa)
hexamethylmelamine
(HMM, altretamine)
Alkyl sulfonates
busulfan
pipobroman
Triazines
dacarbazine (DTIC)
Antimetabolites
Folic Acid analogs
methotrexate
trimetrexate
pemetrexed
(Multi-targeted antifolate)
Pyrimidine analogs
5-fluorouracil
fluorodeoxyuridine
gemcitabine
cytosine arabinoside
(AraC, cytarabine)
5-azacytidine
2,2′-difluorodeoxy-cytidine
floxuridine
pentostatine
Purine analogs
6-mercaptopurine
6-thioguanine
azathioprine
2′-deoxycoformycin
(pentostatin)
erythrohydroxynonyl-adenine (EHNA)
fludarabine phosphate
2-chlorodeoxyadenosine
(cladribine, 2-CdA)
Type I Topoisomerase Inhibitors
camptothecin
topotecan
irinotecan
Biological response modifiers
G-CSF
GM-CSF
Differentiation Agents
retinoic acid derivatives
Hormones and antagonists
Adrenocorticosteroids/antagonists
prednisone and equivalents
dexamethasone
ainoglutethimide
Progestins
hydroxyprogesterone caproate
medroxyprogesterone acetate
megestrol acetate
Estrogens
diethylstilbestrol
ethynyl estradiol/equivalents
Antiestrogen
tamoxifen
Androgens
testosterone propionate
fluoxymesterone/equivalents
Antiandrogens
flutamide
gonadotropin-releasing
hormone analogs
leuprolide
Natural products
Antimitotic drugs
Taxanes
paclitaxel
Vinca alkaloids
vinblastine (VLB)
vincristine
vinorelbine
vindesine
Taxotere ® (docetaxel)
estramustine
estramustine phosphate
Epipodophylotoxins
etoposide
teniposide
Antibiotics
actimomycin D
daunomycin (rubidomycin)
doxorubicin (adriamycin)
mitoxantroneidarubicin
bleomycin
splicamycin (mithramycin)
mitromycin-C
dactinomycin
aphidicolin
epirubicin
idarubicin
daunorubicin
mithramycin
deoxy co-formycin
Enzymes
L-asparaginase
L-arginase
Radiosensitizers
metronidazole
misonidazole
desmethylmisonidazole
pimonidazole
etanidazole
nimorazole
RSU 1069
EO9
RB 6145
Nonsteroidal antiandrogens
SR4233
flutamide
nicotinamide
5-bromodeozyuridine
5-iododeoxyuridine
bromodeoxycytidine
Miscellaneous agents
Platinium coordination complexes
cisplatin
carboplatin
oxaliplatin
anthracenedione
mitoxantrone
Substituted urea
hydroxyurea
Methylhydrazine derivatives
N-methylhydrazine (MIH)
procarbazine
Adrenocortical suppressant
mitotane (o,p′-DDD)
ainoglutethimide
Cytokines
interferon (α, β, γ)
interleukin-2
Photosensitizers
hematoporphyrin derivatives
PHOTOFRIN ®
benzoporphyrin derivatives
Npe6
tin etioporphyrin (SnET2)
pheoboride-a
bacteriochlorophyll-a
naphthalocyanines
phthalocyanines
zinc phthalocyanines
Radiation
X-ray
ultraviolet light
gamma radiation
visible light
infrared radiation
microwave radiation

Microtubule affecting agents interfere with cellular mitosis and are well known in the art for their cytotoxic activity. Microtubule affecting agents useful in the invention include, but are not limited to, allocolchicine (NSC 406042), halichondrin B (NSC 609395), colchicines (NSC 757), colchicines derivatives (e.g., NSC 33410), dolastatin 10 (NSC 376128), maytansine (NSC 153858), rhizoxin (NSC 332598), paclitaxel (NSC 125973), TAXOL® derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, eopthilone B, and discodeiniolide (see Service, (1996) Science, 274:2009) estramustine, nocodazole, MAP4, and the like. Examples of such agents are also described in Bulinski (1997) J. Cell Sci. 110:3055 3064; Panda (1997) Proc. Nat'l. Acad. Sci. USA 94:10560-10564; Muhlradt (1997) Cancer Res. 57:3344-3346; Nicolaou (1997) Nature 397:268-272; Vasquez (1997) Mol. Biol. Cell. 8:973-985; and Panda (1996) J. Biol. Chem. 271:29807-29812.

Cytostatic agents that may be used include, but are not limited to, hormones and steroids (including synthetic analogs): 17-α-ethinylestadiol, diethylstilbestrol, testosterone, prednisone, fluoxymesterone, dromostanolone propionate, testolactone, megestrolacetate, methylprednisolone, methyl-testosterone, prednisolone, triamcinolone, hlorotrianisene, hydroxyprogesterone, aminogluthimide, estramustine, medroxyprogesteroneacetate, leuprolide, flutamide, toremifene, zoladex.

Other cytostatic agents are antiangiogenics such as matrix metalloproteinase inhibitors, and other VEGF inhibitors, such as anti-VEGF antibodies and small molecules such as ZD6474 and SU668. Anti-Her2 antibodies also may be utilized. An EGFR inhibitor is EKB-569 (an irreversible inhibitor). Also included are antibody C225 immunospecific for the EGFR and Src inhibitors.

Also suitable for use as a cytostatic agent is CASODEX® (bicalutamide, Astra Zeneca) which renders androgen-dependent carcinomas non-proliferative. Yet another example of a cytostatic agent is the antiestrogen TAMOXIFEN® which inhibits the proliferation or growth of estrogen dependent breast cancer. Inhibitors of the transduction of cellular proliferative signals are cytostatic agents. Representative examples include epidermal growth factor inhibitors, Her-2 inhibitors, MEK-1 kinase inhibitors, MAPK kinase inhibitors, PI3 inhibitors, Src kinase inhibitors, and PDGF inhibitors.

The compounds of the present invention typically are administered in admixture with a pharmaceutical carrier selected with regard to the intended route of administration and standard pharmaceutical practice. Pharmaceutical compositions for use in accordance with the present invention are formulated in a conventional manner using one or more physiologically acceptable carriers comprising excipients and auxiliaries that facilitate processing of compounds of structural formula (I) or (II).

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the compound of structural formula (I) or (II) is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a compound of structural formula (I) or (II). When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a compound of structural formula (I) or (II).

When a therapeutically effective amount of a compound of structural formula (I) or (II) is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle. A compound of structural formula (I) or (II) can be infused with other fluids over a 10-30 minute span or over several hours.

Compounds of structural formula (I) or (II) can be readily combined with pharmaceutically acceptable carriers well-known in the art. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the compound of structural formula (I) or (II) to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

A compound of structural formula (I) or (II) can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a compound of structural formula (I) or (II) can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

A compound of structural formula (I) or (II) also can be formulated in rectal compositions, such as suppositories or retention enemas, e.g., containing conventional suppository bases. In addition to the formulations described previously, the compound of structural formula (I) or (II) also can be formulated as a depot preparation. Such long-acting formulations can be administered by implantation (for example, subcutaneously or intramuscularly) or by intramuscular injection. Thus, for example, the compounds of structural formula (I) or (II) can be formulated with suitable polymeric or hydrophobic materials (for example, as an emulsion in an acceptable oil) or ion exchange resins.

In particular, the compounds of structural formula (I) or (II) can be administered orally, buccally, or sublingally in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The compounds of structural formula (I) or (II) also can be injected parenterally, for example, intravenously, intramuscularly, subcutaneously, or intracoronarily. For parenteral administration, the STAT3 inhibitors are best used in the form of a sterile aqueous solution which can contain other substances, for example, salts or monosaccharides, such as mannitol or glucose, to make the solution isotonic with blood.

As an additional embodiment, the present invention includes kits which comprise one or more compounds or compositions packaged in a manner that facilitates their use to practice methods of the invention. In one simple embodiment, the kit includes a compound or composition described herein as useful for practice of a method (e.g., a composition comprising a compound of structural formula (I) or (II) and an optional second therapeutic agent), packaged in a container, such as a sealed bottle or vessel, with a label affixed to the container or included in the kit that describes use of the compound or composition to practice the method of the invention. Preferably, the compound or composition is packaged in a unit dosage form. The kit further can include a device suitable for administering the composition according to the intended route of administration.

Prior STAT3 inhibitors possessed properties that hindered their development as therapeutic agents. In accordance with an important feature of the present invention, compounds of structural formula (I) and (II) were synthesized and evaluated as inhibitors for STAT3. For example, compounds of the present invention typically have a bonding affinity (IC₅₀) to STAT3 of less than 100 μM, less than 25 μM, less than 10 μM, and less than 1 μM.

### Synthesis of Compounds

Compounds of the present invention and were prepared as follows.

Unless otherwise stated all temperatures are in degrees Celsius. Also, in these examples and elsewhere, abbreviations have the following meanings:

| | |
|---|---|
| C₆H₅ | phenyl |
| tBu | tertiary butyl |
| Boc | tert-butoxycarbonyl |
| Pd/C | palladium on carbon |
| H₂ | hydrogen gas |
| EtOH | ethanol |
| HCl | hydrochloric acid |
| Cbz | benzyloxycarbonyl |
| NaHCO₃ | sodium bicarbonate |
| LiOH | lithium hydroxide |
| EDCI | 1-(3-dimethylaminopropyl)-3-ethylcarbodimide |
| | hydrochloride |
| HOBT | 1-hydroxybenzotriazole |
| DIPEA | diisopropylethylamine |
| DCM | dichloromethane |
| MeOH | methyl alcohol |
| TFA | trifluoroacetic acid |
| TES | N-[tris(hydroxymethyl)methyl]-2-aminoethanesulfonic |
| | acid |
| NaBH(OAc)₃ | sodium triacetoxyborohydride |
| CH₃CN | acetonitrite |
| K₂CO₃ | potassium carbonate |
| TMSBr | trimethylsilyl bromide |
| Fmoc | 9-fluorenylmethoxycarbonyl |
| Tr | triphenylmethyl (trityl) |
| CDCl₃ | deuterated chloroform |
| CH₃OH—Cl₄ | deuterated methanol |
| and MeOH-d₄ | |
| m | multiplet |
| s | singlet |
| MHz | megahertz |
| δ | chemical shift |
| μM | micromolar |
| J | coupling constant in Hertz |
| dd | doublet of doublets |
| d | doublet |

Solvents and reagents were obtained commercially and used without further purification. NMR spectra were recorded on a Bruker Avance300 spectrometer (300 MHz). Chemical shifts (δ) are reported as δ values (ppm) downfield relative to an internal standard, with multiplicities reported in the usual manner.

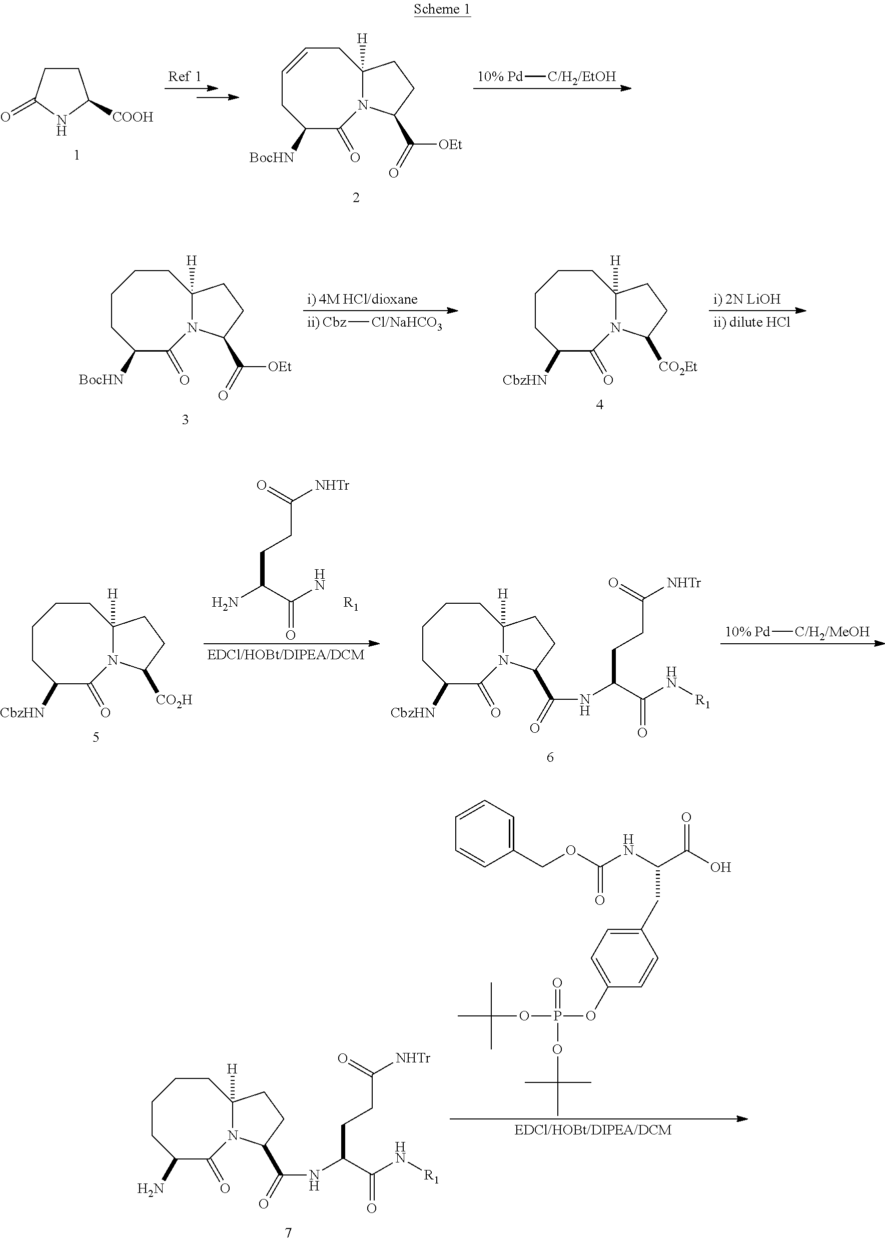

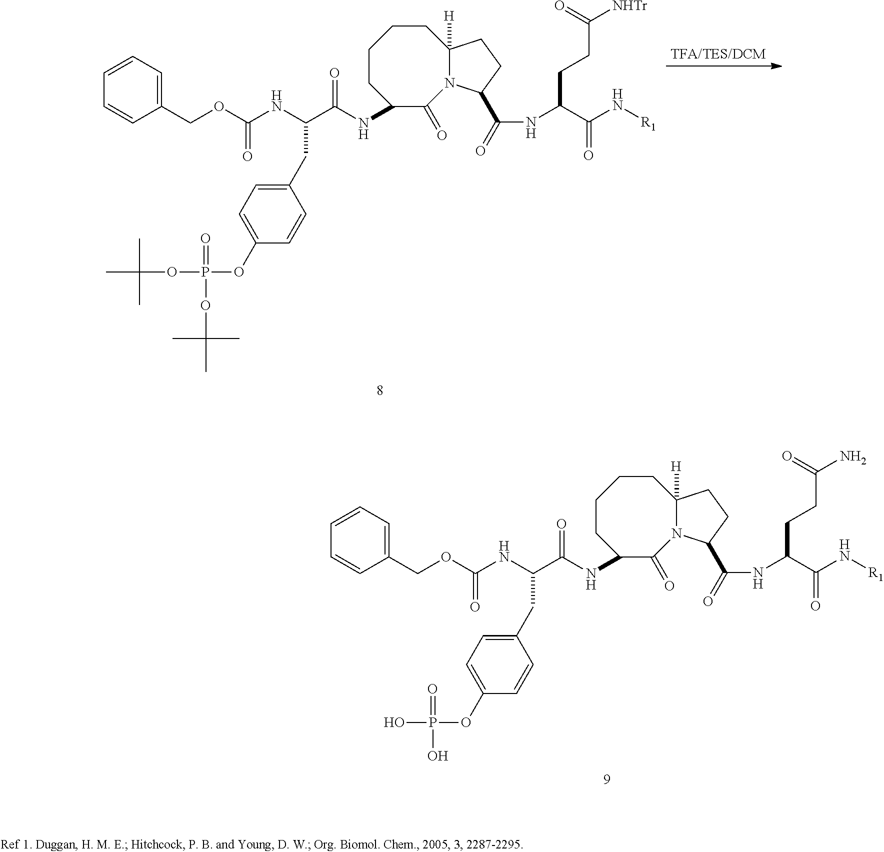

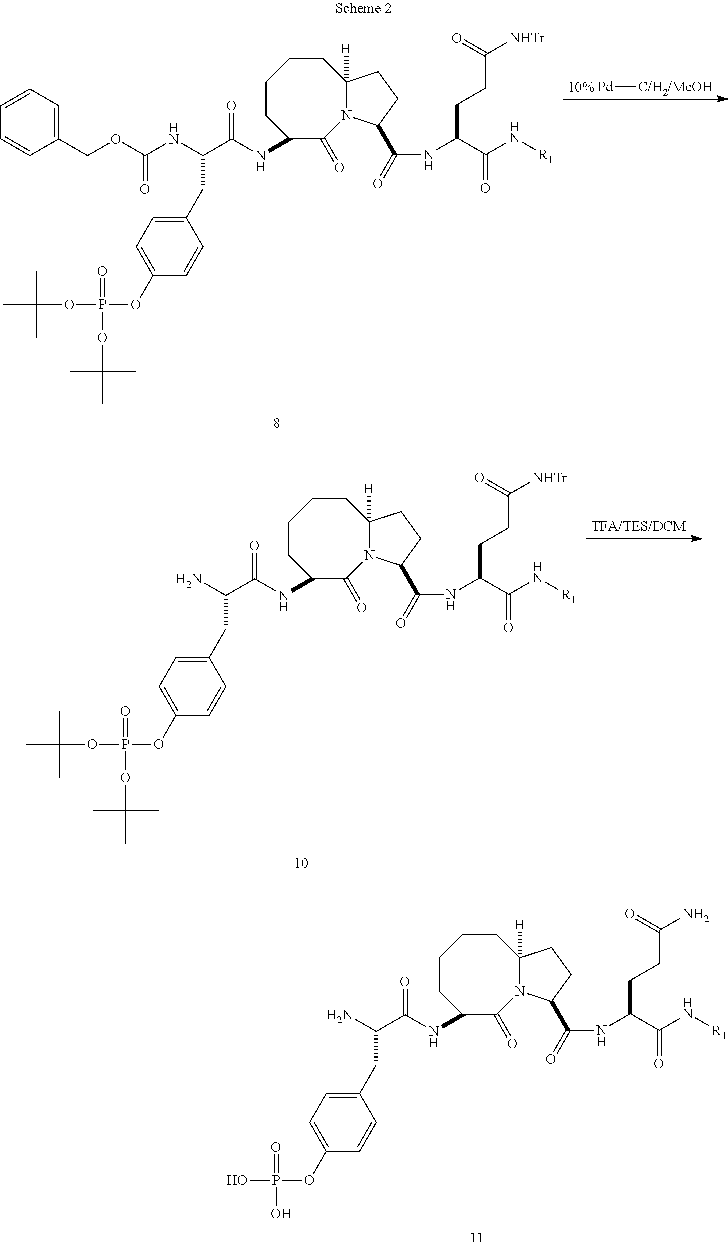

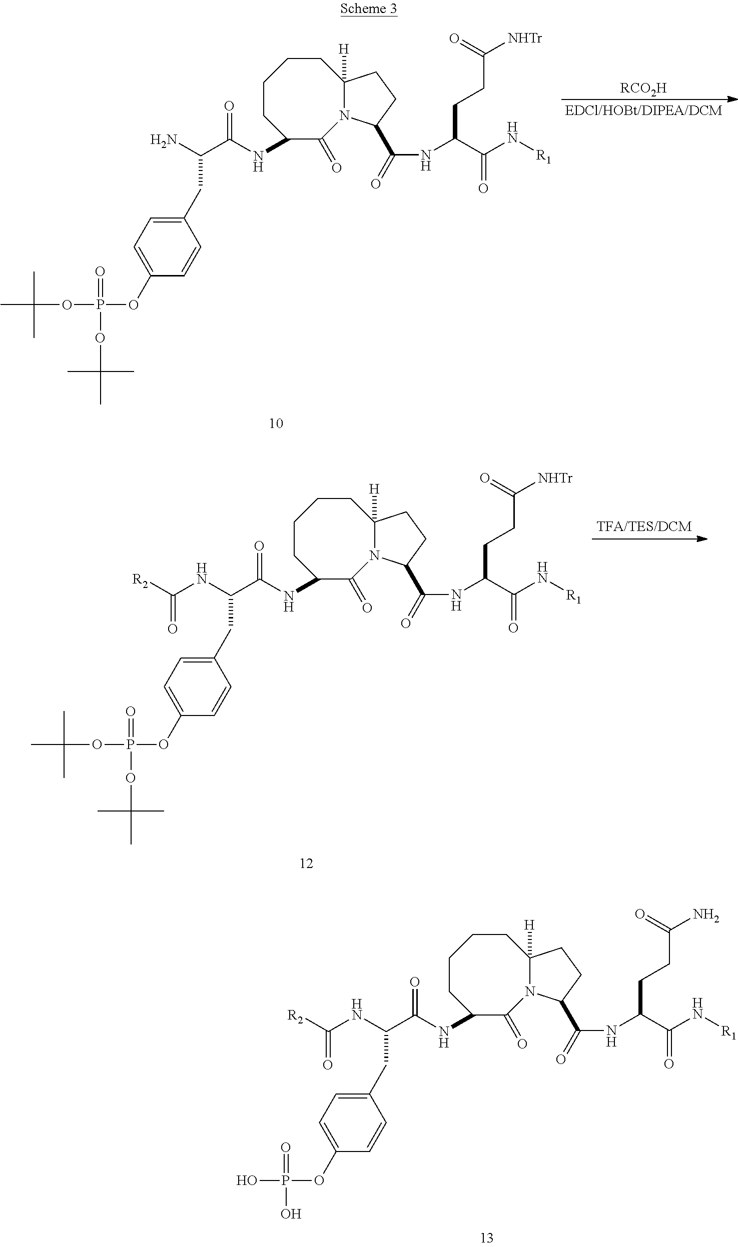

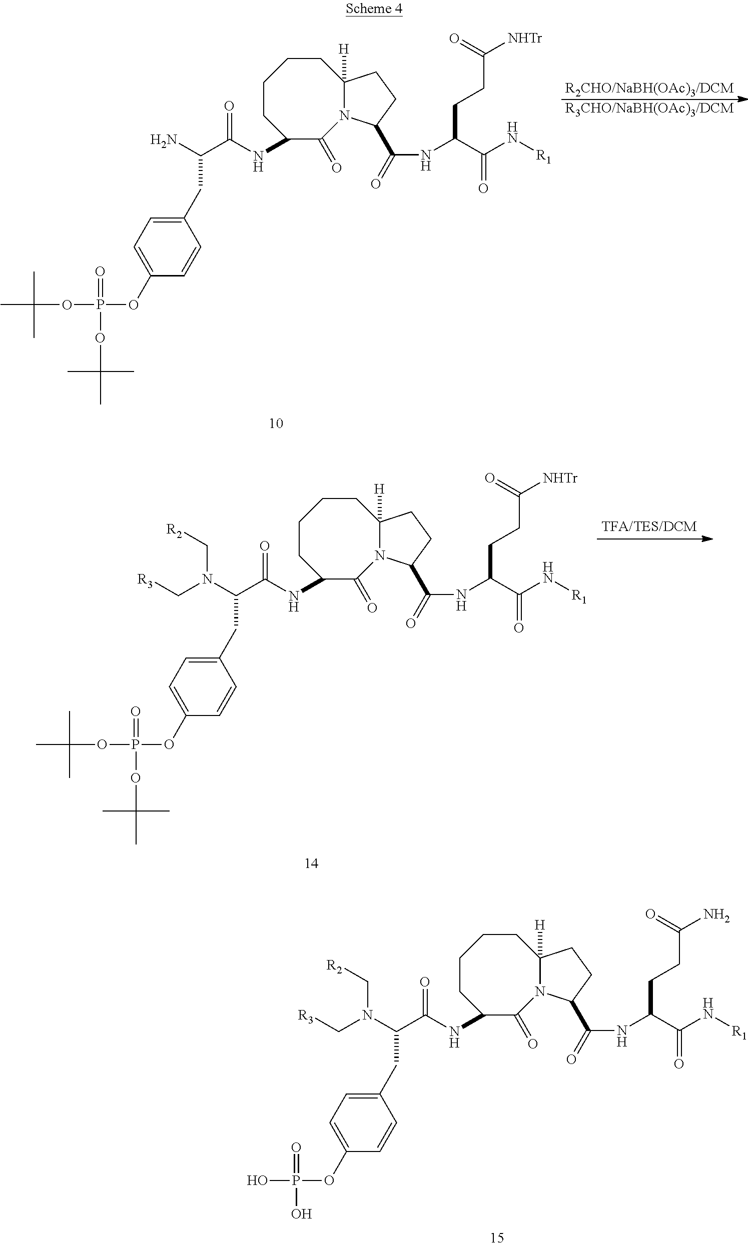

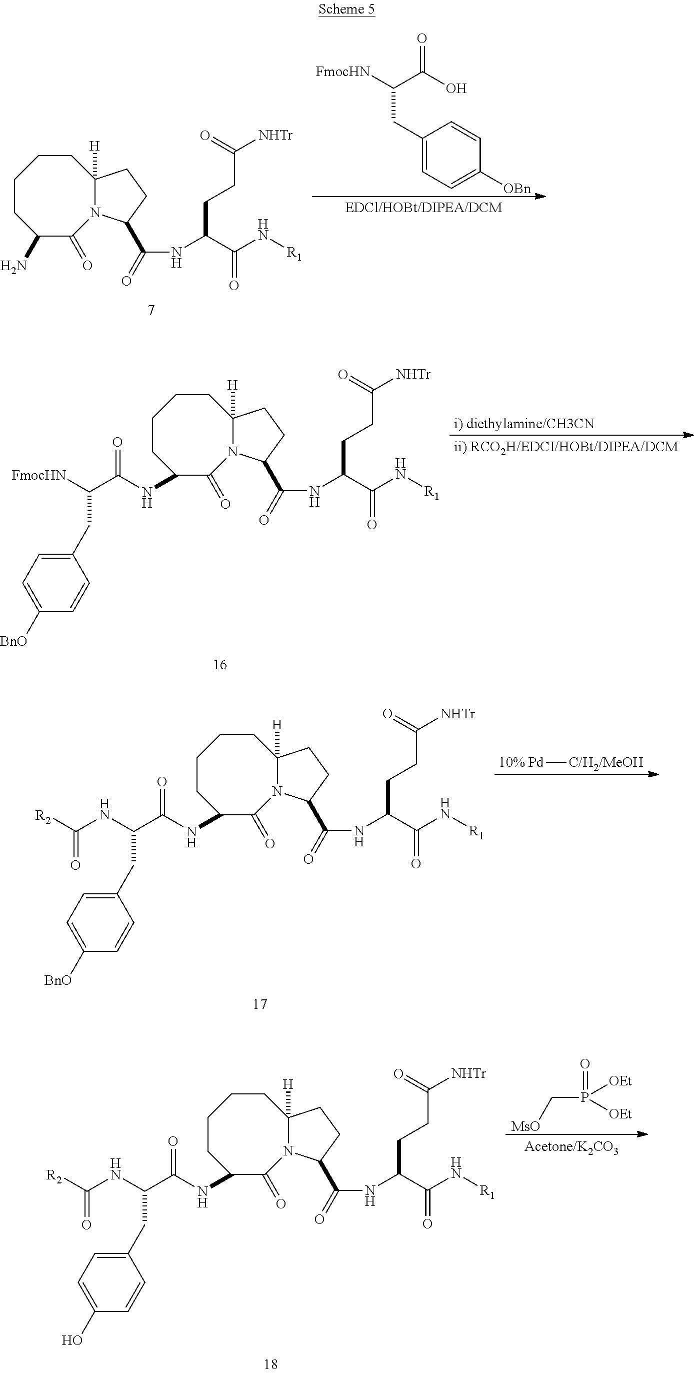

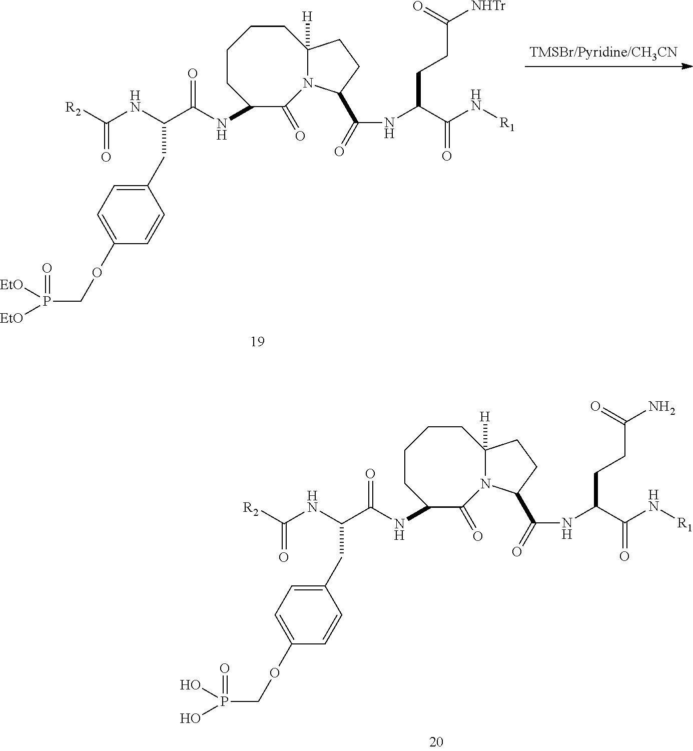

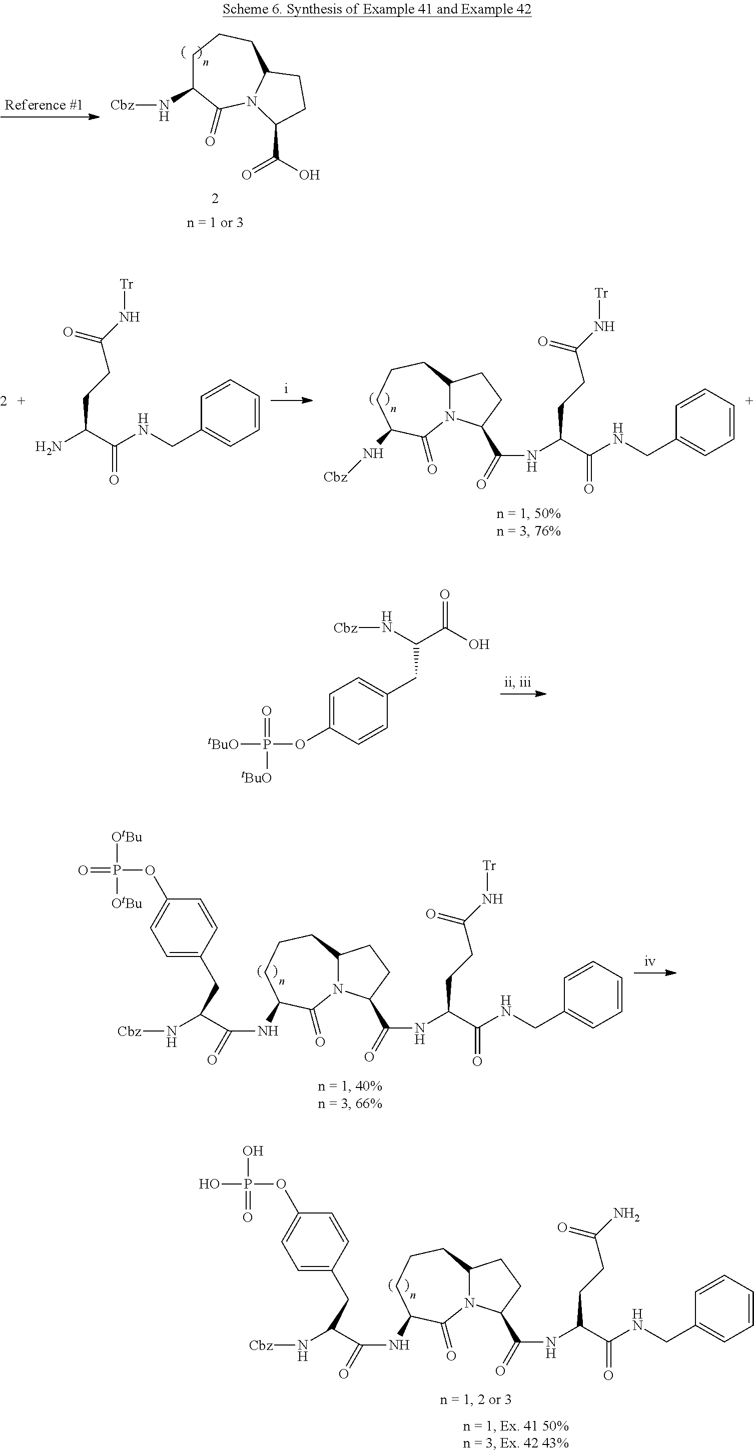

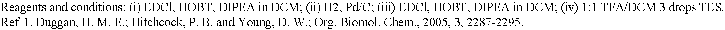

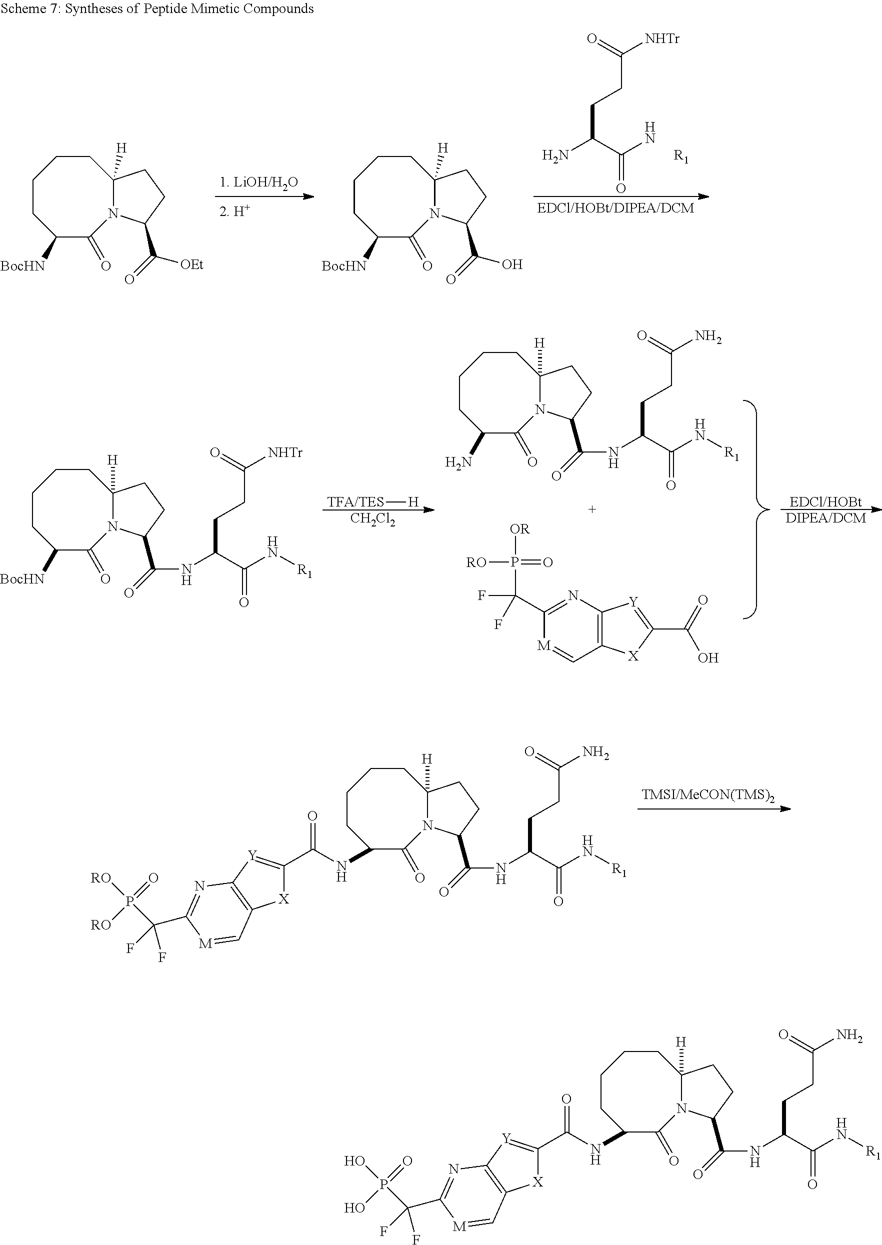

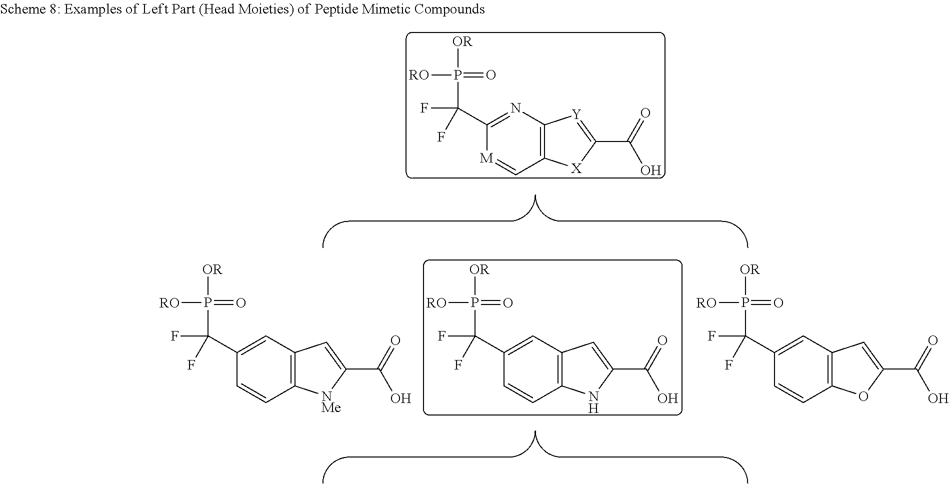

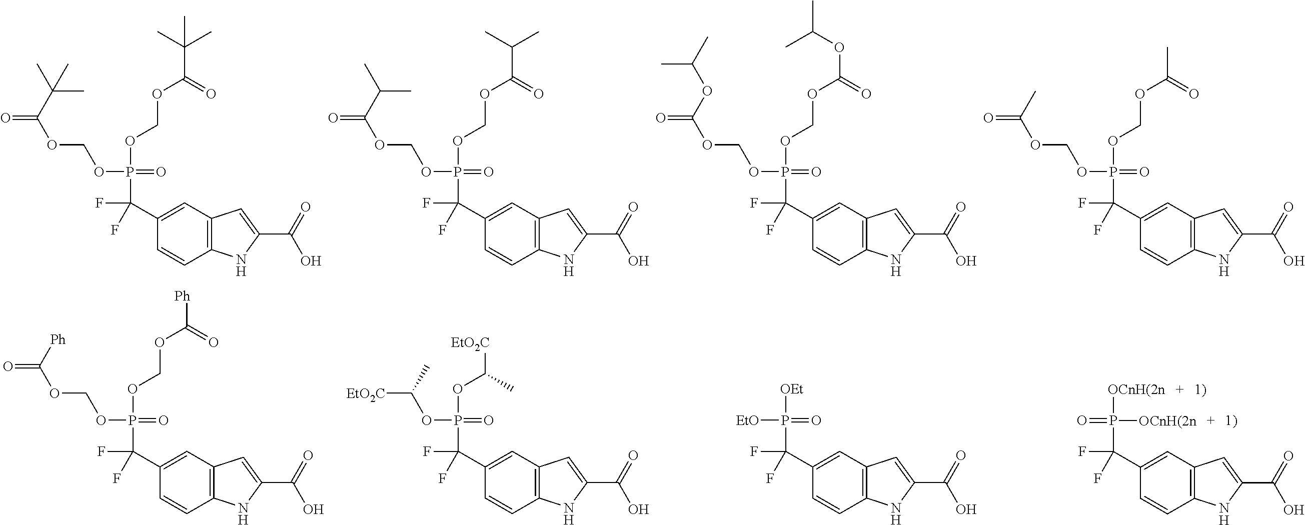

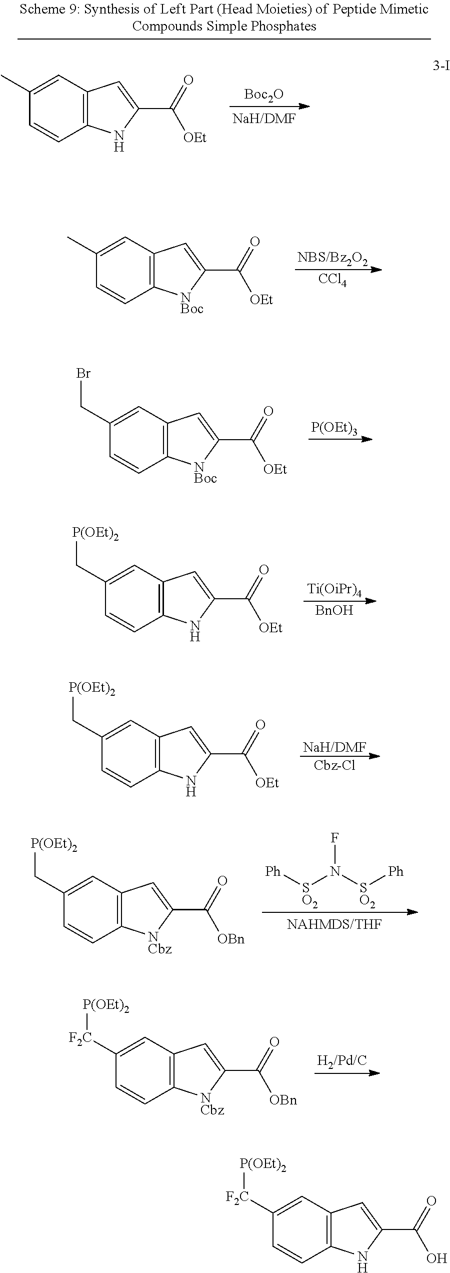

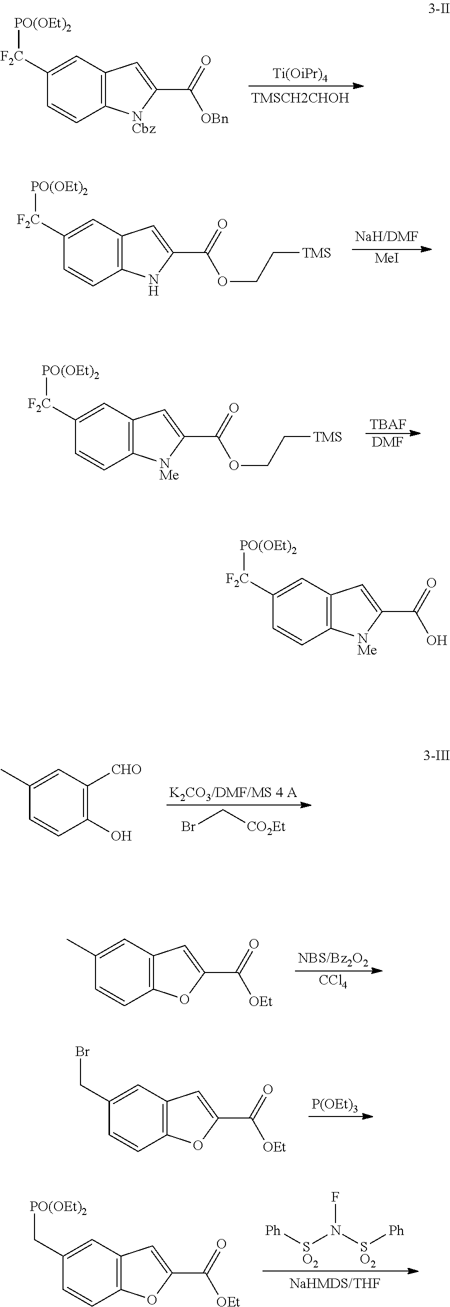

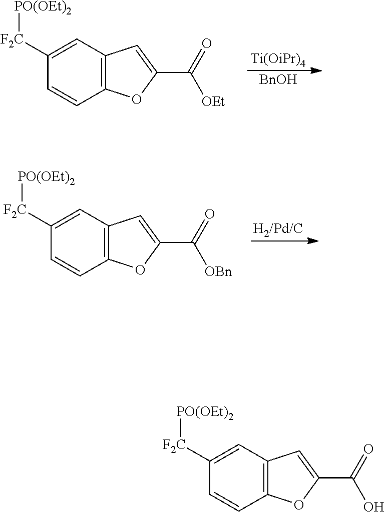

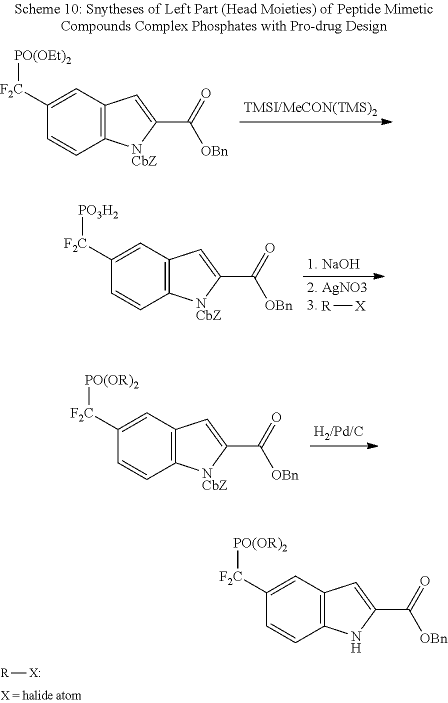

Additional regents and intermediates:

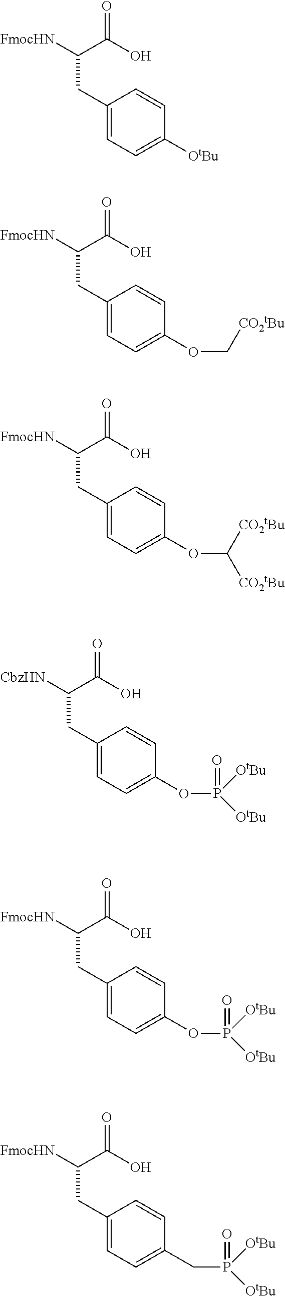

### Example 1

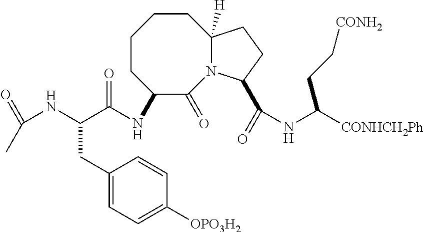

Analytical data: ¹H NMR (300 MHz, MeOH-d₄) δ ppm 7.40-7.20 (m, 7H), 7.18-7.08 (m, 2H), 4.90-4.80 (m, 1H), 4.78-4.55 (m, 1H), 4.50-4.20 (m, 5H), 3.20-3.02 (m, 1H), 2.90-2.75 (m, 1H), 2.55-2.12 (m, 6H), 2.10-1.50 (m, 10H), 1.90 (s, 3H); ¹³C NMR (75 MHz, MeOH-d4) δ ppm 173.4, 172.7, 172.0, 171.4, 138.8, 130.4, 128.5, 127.5, 127.2, 120.2, 61.9, 60.5, 54.9, 53.2, 50.1, 43.0, 37.1, 36.3, 34.4, 32.5, 31.6, 28.3, 27.6, 25.7, 22.4, 21.3.

### Example 2

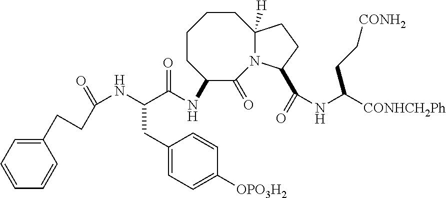

Analytical data: ¹H NMR (300 MHz, MeOH-d₄) δ ppm 7.50-7.00 (m, 14H), 4.85-4.70 (m, 1H), 4.65-4.52 (m, 1H), 4.50-4.20 (m, 5H), 3.10-2.95 (m, 2H), 2.90-2.73 (m, 4H), 2.50-2.08 (m, 6H), 2.07-1.50 (m, 10H); ¹³C NMR (75 MHz, MeOH-d4) δ ppm 175.2, 174.5, 173.7, 172.9, 172.4, 142.1, 139.8, 135.3, 131.4, 129.6, 129.5, 129.4, 128.5, 128.3, 127.2, 121.3, 63.0, 61.5, 55.8, 54.2, 51.1, 44.1, 38.6, 38.1, 37.4, 35.5, 33.5, 32.8, 29.3, 28.7, 26.7, 23.4.

### Example 3

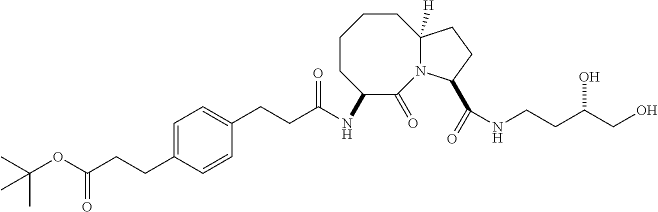

Analytical data: ¹H NMR (300 MHz, CDCl₃) δ ppm 7.10 (s, 4H), 6.93 (t, 1H, J=7.3 Hz), 6.79 (d, 1H, J=7.6 Hz), 7.49-7.87 (m, 1H), 4.43-4.30 (m, 3H), 3.77-3.27 (m, 5H), 3.19-3.06 (m, 1H), 2.96-2.81 (m, 5H), 2.54-2.42 (m, 4H), 2.33-2.02 (m, 4H), 1.93-1.44 (m, 10H), 1.42 (s, 9H); ¹³C NMR (75 MHz, CDCl₃) δ ppm 172.79, 172.39, 171.97, 171.64, 138.59, 128.40, 128.33, 80.39, 69.12, 66.55, 61.45, 59.73, 49.58, 38.02, 37.12, 36.34, 36.18, 35.42, 33.04, 32.36, 31.11, 30.68, 28.07, 26.46, 25.42, 22.66.

### Example 4

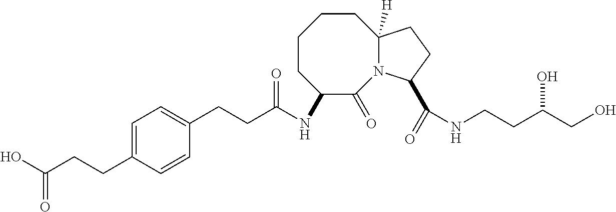

Analytical data: ¹H NMR (300 MHz, CDCl₃) δ ppm 7.20-7.11 (m, 5H), 6.95 (d, 1H, J=7.6 Hz), 5.00-4.87 (m, 2H), 4.35-4.20 (m, 2H), 3.80-3.40 (m, 5H), 3.15-2.80 (m, 5H), 2.70-2.50 (m, 4H), 2.30-2.10 (m, 4H), 2.00-1.30 (m, 10H); ¹³C NMR (75 MHz, CDCl₃) δ ppm 176.08, 173.14, 172.66, 138.32, 138.28, 128.58, 128.54, 69.28, 66.12, 61.85, 60.21, 49.54, 38.13, 36.09, 35.54, 35.09, 32.69, 32.43, 31.46, 30.42, 26.95, 25.37, 22.37.

### Example 5

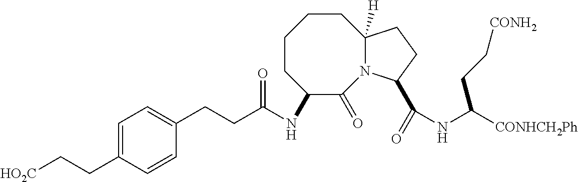

Analytical data: ¹H NMR (300 MHz, CDCl₃) δ ppm 7.85 (d, 1H, J=7.6 Hz), 7.70 (t, J=7.2 Hz, 1H), 7.30-6.50 (m, 12H), 4.95-4.80 (m, 1H), 4.70-4.50 (m, 1H), 4.45-4.15 (m, 4H), 2.90-2.75 (m, 4H), 2.65-1.20 (m, 20H); ¹³C NMR (75 MHz, CDCl₃) δ ppm 177.69, 176.63, 173.14, 172.79, 172.39, 172.23, 138.74, 137.89, 129.08, 128.82, 127.95, 127.79, 62.17, 60.48, 52.58, 49.97, 43.89, 38.08, 36.48, 35.70, 35.33, 32.82, 31.83, 31.66, 30.59, 29.37, 27.61, 25.76, 22.66.

### Example 6

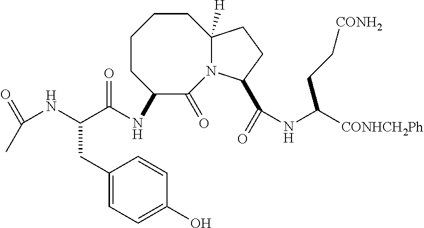

Analytical data: ¹H NMR (300 MHz, CH₃OH-d₄) δ ppm 7.32-7.18 (m, 5H), 7.03 (d, 2H, J=8.6 Hz), 6.67 (d, J=8.6 Hz, 2H), 4.80 (dd, 1H, J=12.2Hz, 5.0 Hz), 4.53 (dd, 1H, J=9.5 Hz, 5.2 Hz), 4.43-4.22 (m, 5H), 2.98 (dd, 1H, J=14.0 Hz, 5.2 Hz), 2.72 (dd, 1H, J=9.5 Hz, 14.0 Hz), 2.48-2.05 (m, 5H), 2.00-1.55 (m, 11H), 1.87 (s, 3H); ¹³C NMR (75 MHz, CH₃OH-d₄) δ ppm 178.88, 175.21, 174.48, 174.18, 174.04, 173.35, 158.11, 140.65, 132.08, 130.40, 130.02, 129.35, 129.09, 117.03, 63.79, 62.31, 57.10, 52.04, 44.93, 38.93, 38.19, 36.36, 34.34, 33.43, 30.19, 29.50, 27.52, 24.26, 23.21.

### Example 7

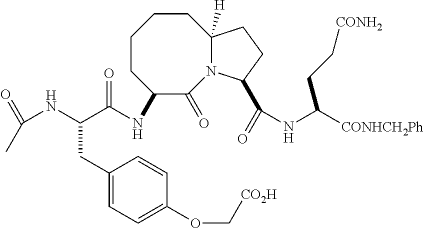

Analytical data: ¹H NMR (300 MHz, CH₃OH-d₄) δ ppm 7.32-7.20 (m, 5H), 7.15 (d, 2H, J=8.6 Hz), 6.82 (d, 2H, J8.6 Hz), 4.82 (dd, 1H, J=11.9 Hz, 4.6 Hz), 4.63 (s, 2H), 4.56 (dd, 1H, J=9.2 Hz, 5.4 Hz), 4.46-4.32 (m, 5H), 2.50-1.52 (m, 16H), 1.90 (s, 3H); ¹³C NMR (75 MHz, CH₃OH-d₄) δ ppm 177.08, 173.38, 172.62, 172.20, 172.14, 171.84, 171.45, 157.34, 138.74, 130.38, 130.34, 128.55, 127.48, 127.23, 64.99, 61.92, 60.46, 55.08, 53.10, 50.16, 43.08, 37.01, 36.32, 34.47, 32.50, 31.56, 28.31, 27.64, 25.64, 22.37, 21.38.

### Example 8

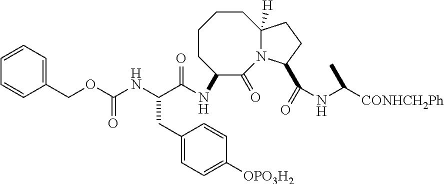

Analytical data: ¹H NMR (300 MHz, CH₃OH-d₄) δ ppm 7.40-7.15 (m, 12H), 7.09 (d, 2H, J=8.6 Hz), 5.02 (s, 2H), 4.90-4.80 (m, 1H), 4.45-4.25 (m, 6H), 3.10 (dd, 1H, J=14.0 Hz, 4.3 Hz), 2.80 (dd, J=14.0 Hz, 10 Hz), 2.30-1.50 (m, 12H), 1.42 (d, 3H, J=7.2 Hz); ¹³C NMR (75 MHz, CH₃OH-d₄) δ ppm 173.95, 173.04, 172.50, 171.47, 157.27, 150.60, 138.87, 137.08, 134.01, 130.53, 128.49, 127.97, 127.71, 127.41, 127.16, 120.20, 66.59, 61.70, 60.40, 56.57, 43.00, 36.37, 34.47, 32.48, 27.54, 25.66, 22.43, 16.99.

### Example 9

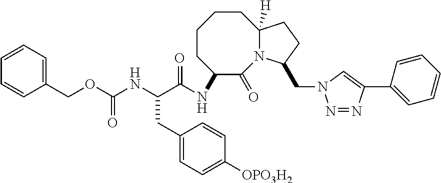

Analytical data: ¹H NMR (300 MHz, CH₃OH-d₄) δ ppm 8.37 (s, 1H), 7.81 (d, 2H, J=7.1 Hz), 7.45-7.15 (m, 10H), 7.12 (d, 2H, J=7.1 Hz), 5.01 (s, 2H), 4.90-4.15 (m, 6H), 3.20-3.10 (m, 1H), 2.85-2.70 (m, 1H), 2.30-1.30 (m, 12H); ¹³C NMR (75 MHz, CH₃OH-d₄) δ ppm 172.46, 172.37, 157.28, 150.53, 147.74, 137.09, 134.08, 130.53, 129.02, 128.48, 128.43, 127.97, 127.68, 125.69, 122.32, 120.30, 120.23, 66.58, 59.92, 58.95, 56.65, 52.65, 50.49, 37.94, 37.31, 31.62, 27.15, 25.37, 2324.

### Example 10

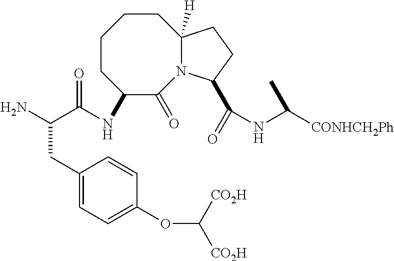

Analytical data: ¹H NMR (300 MHz, CH₃OH-d₄) δ ppm 8.3 (t, 1H), 7.25-7.00 (m, 7H), 6.80 (d, 2H, J=7.8 Hz), 5.20 (s, 1H), 4.40-3.80 (m, 6H), 3.20-2.70 (m, 2H), 2.25-1.35 (m, 12H), 1.27 (d, 3H, J=7.1 Hz).

### Example 11

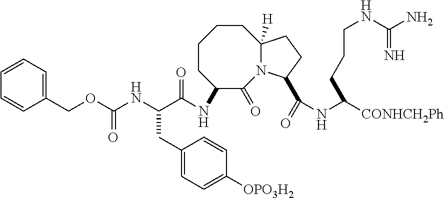

Analytical data: ¹H NMR (300 MHz, CH₃OH-d₄) δ ppm 8.40 (t, 1H), 7.75 (d, 1H), 7.45-7.00 (m, 14H), 5.05 (s, 2H), 4.80-4.65 (m, 1H), 4.55-4.15 (m, 6H), 3.30-2.80 (m, 4H), 2.35-1.50 (m, 16H).

### Example 12

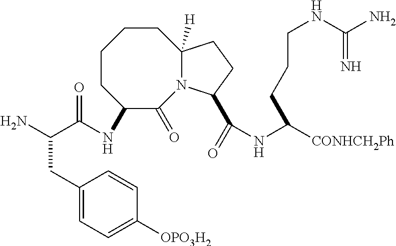

Analytical data: ¹H NMR (300 MHz, CH₃OH-d₄) δ ppm 7.50-7.00 (m, 9H), 4.60-3.95 (m, 7H), 3.30-2.80 (m, 4H), 2.40-1.50 (m, 16H).

### Example 13

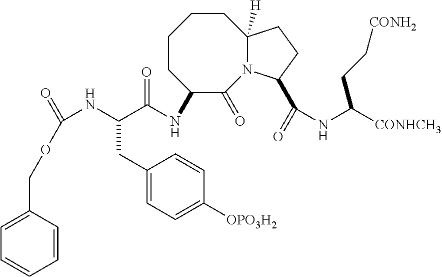

Analytical data: ¹H NMR (300 MHz, CH₃OH-d₄) δ ppm 7.40-7.00 (m, 9H), 5.01 (s, 2H), 4.92-4.80 (m, 1H), 4.50-4.20 (m, 4H), 3.20-2.70 (m, 2H), 2.71 (s, 3H), 2.50-1.45 (m, 16H).

### Example 14

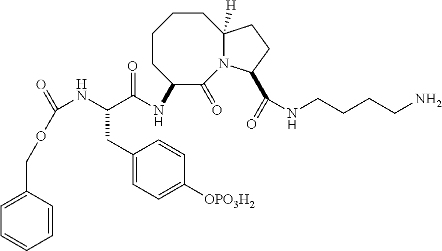

Analytical data: ¹H NMR (300 MHz, CH₃OH-d₄) δ ppm 7.40-7.01 (m, 9H), 5.03 (s, 2H), 4.92-4.78 (m, 1H), 3.45-3.25 (m, 3H), 3.20-2.70 (m, 6H), 2.35-1.50 (m, 16H).

### Example 15

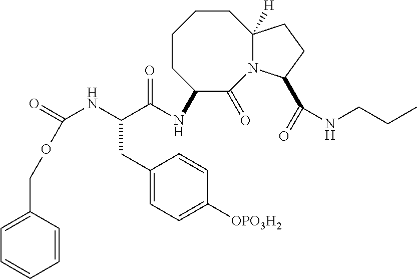

Analytical data: ¹H NMR (300 MHz, CH₃OH-d₄) δ ppm 7.40-7.15 (m, 7H), 7.10 (d, 2H, J=8.14 Hz), 5.01 (s, 2H), 4.90-4.80 (m, 1H), 4.53-4.20 (m, 3H), 3.25-3.04 (m, 3H), 2.80 (dd, 1H, J=10.2 Hz, 3.4 Hz), 2.30-1.42 (m, 14H), 0.95 (t, 3H, J=7.3 Hz); ¹³C NMR (75 MHz, CH₃OH-d₄) δ ppm 173.23, 172.52, 171.35, 157.28, 137.09, 134.13, 130.57, 130.36, 128.49, 127.97, 127.69, 120.25, 120.19, 67.14, 62.01, 60.48, 56.56, 50.14, 41.30, 37.46, 36.24, 34.57, 32.49, 27.87, 25.71, 22.62, 22.33, 10.78.

### Example 16

Analytical data: ¹H NMR (300 MHz, CH₃OH-d₄) δ ppm 7.40-7.00 (m, 9H), 5.01 (s, 2H), 4.90-4.78 (m, 1H), 4.50-4.20 (m, 3H), 3.30-3.02 (m, 3H), 2.87-2.70 (m, 1H), 2.35 (t, 2H, J=7.2 Hz), 2.30-1.45 (m, 14H); ¹³C NMR (75 MHz, CH₃OH-d₄) δ ppm 174.32, 173.38, 172.52, 171.35, 157.27, 137.10, 134.11, 130.57, 128.49, 127.98, 127.69, 120.25, 62.02, 60.47, 56.56, 51.09, 50.12, 38.66, 37.47, 36.23, 34.60, 32.49, 30.96, 27.85, 25.70, 24.74, 22.34.

### Example 17

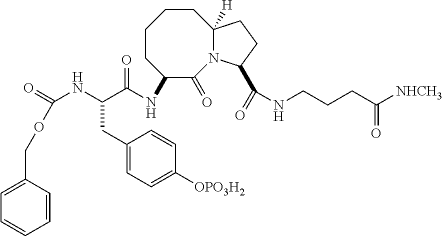

Analytical data: ¹H NMR (300 MHz, CH₃OH-d₄) δ ppm 7.40-7.15 (m, 7H), 7.10 (d, 2H, J=8.0 Hz), 5.02 (s, 2H), 4.92-4.75 (m, 1H), 4.50-4.25 (m, 3H), 3.25-3.05 (m, 2H), 2.80 (s, 3H), 2.60-1.50 (m, 16H); ¹³C NMR (75 MHz, CH₃OH-d₄) δ ppm 176.72, 174.05, 172.54, 171.61, 157.26, 137.09, 134.07, 130.63, 128.53, 128.03, 127.71, 120.27, 66.61, 62.19, 60.57, 56.57, 50.21, 38.24, 37.41, 36.30, 34.43, 32.55, 31.66, 27.91, 26.61, 25.95, 25.70, 22.28.

### Example 18

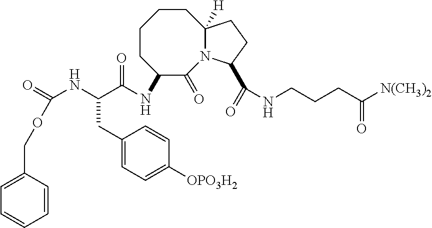

Analytical data: ¹H NMR (300 MHz, CH₃OH-d₄) δ ppm 7.40-7.14 (m, 7H), 7.10 (d, 2H, J=7.7 Hz), 5.02 (s, 2H), 4.90-4.70 (m, 1H), 4.55-4.20 (m, 3H), 3.30-2.95 (m, 8H), 2.90-2.50 (m, 3H), 2.40-1.45 (m, 14H); ¹³C NMR (75 MHz, CH₃OH-d₄) δ ppm 175.42, 174.14, 172.52, 171.46, 157.27, 137.11, 134.14, 130.66, 128.56, 128.05, 127.70, 120.29, 66.61, 62.21, 60.49, 56.59, 50.20, 38.72, 38.24, 37.43, 36.29, 34.47, 32.57, 29.39, 27.92, 25.69, 25.28, 22.32.

### Example 19

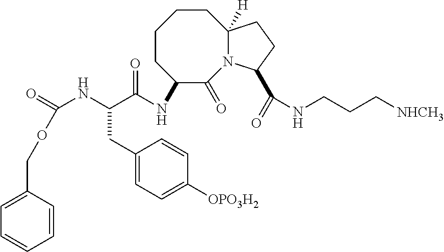

Analytical data: ¹H NMR (300 MHz, CH₃OH-d₄) δ ppm 7.40-7.20 (m, 7H), 7.10 (d, 2H, J=7.6 Hz), 4.90-4.78 (m, 1H), 4.45-4.20 (m, 3H), 3.55-3.45 (m, 1H), 3.25-2.75 (m, 5H), 2.65 (s, 3H), 2.35-1.45 (m, 14H); ¹³C NMR (75 MHz, CH₃OH-d₄) δ ppm 174.83, 172.42, 171.37, 157.25, 137.10, 133.98, 130.59, 128.51, 128.02, 127.69, 120.25, 66.59, 62.09, 60.49, 56.58, 50.09, 46.63, 37.32, 36.33, 35.64, 34.41, 32.92, 32.55, 27.81, 26.63, 25.68, 22.28.

### Example 20

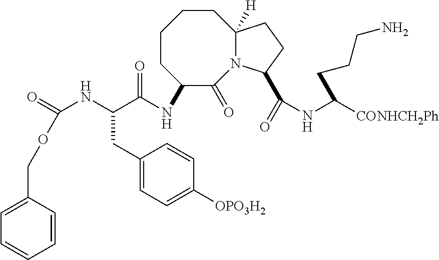

Analytical data: ¹H NMR (300 MHz, CH₃OH-d₄) δ ppm 7.35-7.15 (m, 12H), 7.10 (d, 2H, J=8.2 Hz), 5.01 (m, 2H), 4.92-4.75 (m, 1H), 4.45-4.20 (m, 6H), 3.15-2.75 (m, 4H), 2.30-1.46 (m, 16H).

### Example 21

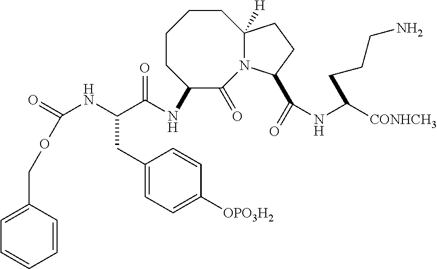

Analytical data: ¹H NMR (300 MHz, CH₃OH-d₄) δ ppm 7.40-7.20 (m, 7H), 7.10 (d, 2H, J=8.0 Hz), 4.90-4.79 (m, 1H), 4.45-4.25 (m, 4H), 3.20-2.80 (m, 4H), 2.75 (s, 3H), 2.40-1.46 (m, 16H).

### Example 22

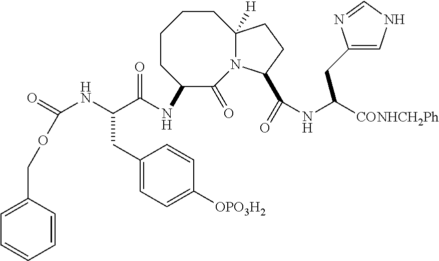

Analytical data: ¹H NMR (300 MHz, CH₃OH-d₄) δ ppm 8.65 (s, 1H), 7.40-7.15 (m, 13H), 7.10 (d, 2H, J=8.2 Hz), 5.02 (s, 2H), 4.85-4.60 (m, 2H), 4.50-4.20 (m, 5H), 3.30-2.80 (m, 4H), 2.35-1.50 (m, 12H).

### Example 23

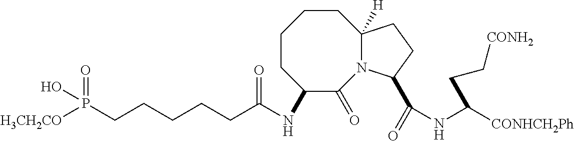

Analytical data: ¹H NMR (300 MHz, CH₃OH-d₄) δ ppm 7.35-7.15 (m, 5H), 4.92-4.75 (m, 1H), 4.50-4.25 (m, 5H), 4.05 (m, 2H), 2.55-1.35 (m, 26H), 1.27 (t, 3H, J=7.0 Hz).

### Example 24

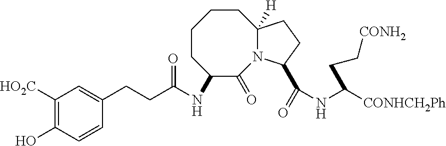

Analytical data: ¹H NMR (300 MHz, CH₃OH-d₄) δ ppm 7.70 (d, 1H, J=2.2 Hz), 7.40-7.19 (m, 6H), 6.80 (d, 1H, J=8.5 Hz), 4.90-4.78 (m, 1H), 4.45-4.20 (m, 5H), 2.90-2.80 (m, 2H), 2.50-2.10 (m, 7H), 2.05-1.40 (m, 11H).

### Example 25

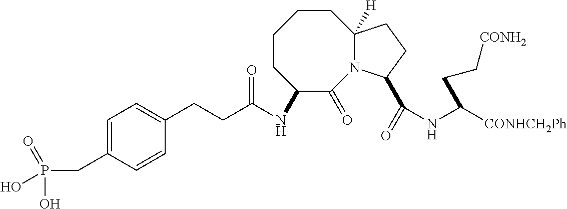

Analytical data: ¹H NMR (300 MHz, CH₃OH-d₄) δ ppm 7.50-7.10 (m, 9H), 4.90-4.75 (m, 1H), 4.50-4.20 (m, 5H), 3.20-2.80 (m, 4H), 2.60-2.35 (m, 4H), 2.30-1.40 (m, 14H).

### Example 26

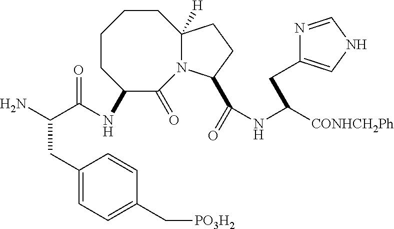

Analytical data: ¹H NMR (300 MHz, CH₃OH-d₄) δ ppm 8.70 (d, 1H, J=1.4 Hz), 8.58 (d, 1H, J=6.8 Hz), 7.40-7.20 (m, 10H), 4.90-4.78 (m, 1H), 4.70-4.55 (m, 1H), 4.50-4.25 (m, 4H), 4.20-4.10 (m, 1H), 3.32-3.20 (m, 3H), 3.12 (d, 2H, J=21.5 Hz), 3.00 (dd, 1H, J=14.2 Hz, 8.2 Hz), 2.35-1.50 (m, 12H); ¹³C NMR (75 MHz, CH₃OH-d₄) δ ppm 173.31, 171.10, 170.57, 168.16, 138.73, 133.78, 133.20, 133.08, 132.77, 132.72, 130.52, 130.43, 130.00, 129.68, 129.64, 128.55, 127.54, 127.31, 117.51, 61.98, 60.66, 54.39, 53.15, 50.24, 43.16, 37.15, 36.27, 35.39, 34.43, 33.61, 32.54, 27.65, 26.70, 25.62, 22.27.

### Example 27

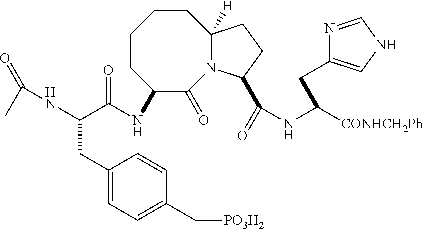

Analytical data: ¹H NMR (300 MHz, CH₃OH-d₄) δ ppm 8.70 (d, 1H, J=1.3 Hz), 7.40-7.10 (m, 10H), 4.90-4.78 (m, 1H), 4.70-4.55 (m, 2H), 4.45-4.25 (m, 4H), 3.35-3.10 (m, 2H), 3.08 (d, 2H, J=21.6 Hz), 2.90-2.80 (m, 1H), 2.30-1.50 (m, 12H), 1.90 (s, 3H); ¹³C NMR (75 MHz, CH₃OH-d₄) δ ppm 173.28, 172.32, 172.14, 171.48, 170.41, 138.75, 135.78, 135.73, 133.83, 131.91, 131.79, 129.98, 129.91, 129.29, 129.25, 128.57, 127.62, 127.36, 117.55, 62.07, 60.49, 54.94, 52.96, 50.23, 43.18, 37.22, 36.29, 35.36, 34.40, 33.57, 32.49, 27.61, 26.71, 25.58, 22.37, 21.39.

### Example 28

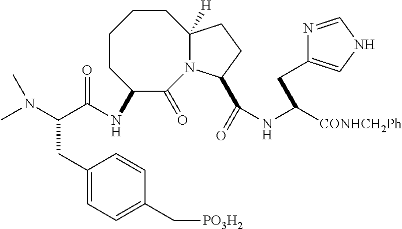

Analytical data: ¹H NMR (300 MHz, CH₃OH-d₄) δ ppm 8.62 (d, 1H, J=1.0 Hz), 8.42 (d, 1H, J=7.2 Hz), 7.35-7.15 (m, 10H), 4.92-4.79 (m, 1H), 4.65-4.50 (m, 1H), 4.45-4.10 (m, 5H), 3.30-3.05 (m, 6H), 2.95 (s, 6H), 2.30-1.50 (m, 12H); ¹³C NMR (75 MHz, CH₃OH-d₄) δ ppm 173.28, 170.67, 170.19, 166.12, 138.72, 133.79, 133.04, 132.93, 132.58, 132.54, 130.61, 130.39, 130.31, 129.96, 129.58, 128.55, 127.51, 127.30, 117.42, 69.07, 61.78, 60.56, 53.22, 50.10, 43.14, 41.40, 36.23, 35.45, 34.69, 34.27, 33.67, 32.51, 27.65, 26.63, 25.45, 22.31.

### Example 29

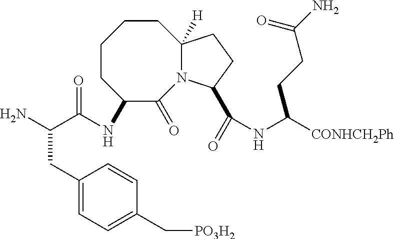

Analytical data: ¹H NMR (300 MHz, CH₃OH-d₄) δ ppm 8.62 (d, 1H, J=6.7 Hz), 7.30-7.15 (m, 9H), 4.50-4.20 (m, 5H), 4.15-4.05 (m, 1H), 3.30-3.20 (m, 1H), 3.10 (d, 2H, J=21.5 Hz), 3.00-2.85 (m, 1H), 2.50-1.45 (m, 16H); ¹³C NMR (75 MHz, CH₃OH-d₄) δ ppm 177.33, 173.56, 172.89, 171.47, 168.48, 139.06, 133.52, 133.39, 133.13, 133.08, 130.87, 130.79, 129.87, 129.84, 128.82, 127.75, 127.51, 62.20, 60.92, 54.85, 53.51, 50.61, 43.91, 37.47, 36.57, 35.66, 34.65, 33.87, 32.84, 31.91, 28.59, 27.96, 25.97, 22.58.

### Example 30

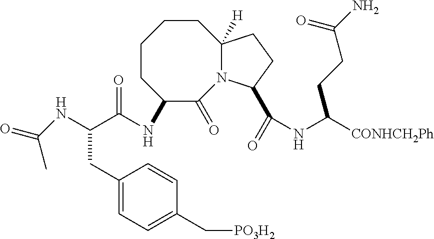

Analytical data: ¹H NMR (300 MHz, CH₃OH-d₄) δ ppm 7.30-7.06 (m, 9H), 4.92-4.80 (m, 1H), 4.65-4.50 (m, 1H), 4.50-4.20 (m, 5H), 3.20-3.00 (m, 1H), 3.08 (d, 2H, J=21.5 Hz), 2.85-2.70 (m, 1H), 2.50-1.45 (m, 16H), 1.85 (s, 3H); ¹³C NMR (75 MHz, CH₃OH-d₄) δ ppm 177.05, 173.36, 172.63, 172.25, 171.61, 138.77, 135.86, 135.81, 131.80, 131.68, 129.96, 129.88, 129.30, 129.26, 128.54, 127.47, 127.22, 61.91, 60.52, 54.89, 53.18, 50.19, 43.05, 37.44, 36.30, 35.36, 34.35, 33.57, 32.51, 31.59, 28.30, 27.66, 25.67, 22.33, 21.36.

### Example 31

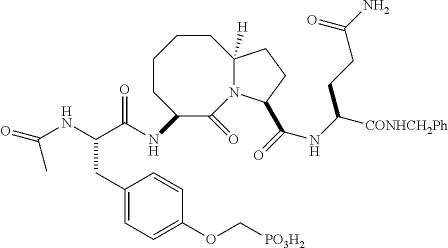

Analytical data: ¹H NMR (300 MHz, CH₃OH-d₄) δ ppm 7.40-7.20 (m, 5H), 7.18 (d, 2H, J=8.7 Hz), 6.92 (d, 2H, J=8.7 Hz), 4.89-4.70 (m, 1H), 4.65-4.22 (m, 6H), 4.20 (d, 2H, J=10.5 Hz), 3.10-2.70 (m, 2H), 2.50-1.40 (m, 16H), 1.90 (s, 3H); ¹³C NMR (75 MHz, CH₃OH-d₄) δ ppm 177.10, 173.38, 172.69, 172.14, 172.06, 171.27, 138.77, 130.40, 130.32, 128.53, 127.47, 127.22, 114.43, 62.13, 61.82, 60.49, 55.28, 53.27, 50.10, 43.04, 37.15, 36.29, 34.56, 32.50, 31.64, 28.29, 27.67, 25.62, 22.33, 21.34.

### Example 32

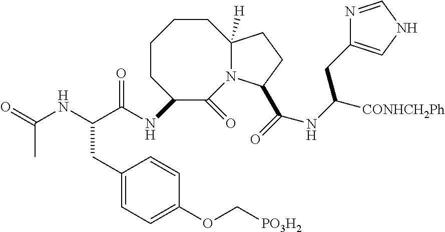

Analytical data: ¹H NMR (300 MHz, CH₃OH-d₄) δ ppm 8.7 (d, 1H, J=1.3 Hz), 7.40-7.20 (m, 6H), 7.15 (d, 2H, J=8.6 Hz), 6.90 (d, 2H, J=8.6 Hz), 4.89-4.70 (m, 1H), 4.65-4.20) (m, 6H), 4.15 (d, 2H, J=10.4 Hz), 3.30-2.80 (m, 4H), 2.50-1.45 (m, 12H), 1.92 (s, 3H); ¹³C NMR (75 MHz, CH₃OH-d₄) δ ppm 73.34, 172.28, 171.93, 171.07, 170.65, 138.72, 133.82, 130.33, 129.97, 128.58, 127.61, 127.35, 117.56, 114.52, 64.59, 62.39, 61.95, 60.48, 55.57, 53.16, 50.13, 43.20, 37.08, 36.28, 34.73, 32.50, 27.62, 26.68, 25.54, 22.38, 21.43.

### Example 33

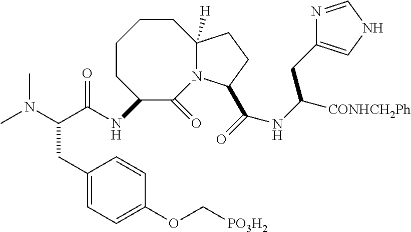

Analytical data: ¹H NMR (300 MHz, CH₃OH-d₄) δ ppm 8.70 (d, 1H, J=1.3 Hz), 7.65 (d, 1H, J=7.5 Hz), 7.30-7.20 (m, 6H), 7.09 (d, 2H, J=8.6 Hz), 6.87 (d, 2H, J=8.6 Hz), 4.90-4.30 (m, 5H), 4.12 (d, 2H, J=10.4 Hz), 4.25-4.00 (m, 2H), 3.34-2.75 (m, 4H), 2.95 (s, 6H), 2.50-1.45 (m, 12H); ¹³C NMR (75 MHz, CH₃OH-d₄) δ ppm 173.65, 171.25, 169.54, 166.18, 138.98, 133.98, 130.70, 130.41, 128.73, 127.73, 127.46, 127.17, 117.72, 115.13, 70.37, 65.30, 63.11, 61.70, 60.28, 60.16, 54.06, 50.06, 43.35, 36.48, 35.65, 34.54, 32.72, 28.03, 26.61, 25.62, 22.59.

### Example 34

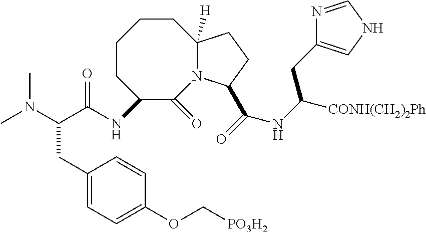

Analytical data: ¹H NMR (300 MHz, CH₃OH-d₄) δ ppm 8.65 (s, 1H), 7.80 (d, 1H, J=7.5 Hz), 7.30-7.15 (m, 6H), 7.10 (d, 2H, J=8.5 Hz), 6.87 (d, 2H, J=8.5 Hz), 4.90-4.72 (m, 2H), 4.50-4.05 (m, 6H), 3.50-2.70 (m, 8H), 3.00 (s, 6H), 2.50-2.10 (m, 2H), 1.90-1.40 (m, 10H); ¹³C NMR (75 MHz, CH₃OH-d₄) δ ppm 173.29, 170.96, 169.32, 166.11, 166.02, 139.35, 133.78, 130.54, 130.02, 128.86, 128.56, 127.04, 126.43, 117.53, 114.91.

### Example 35

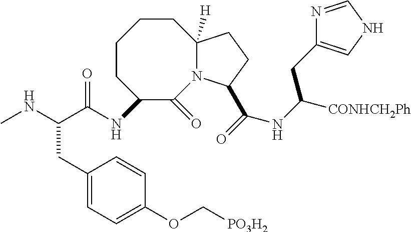

Analytical data: ¹H NMR (300 MHz, CH₃OH-d₄) δ ppm 8.70 (d, 1H, J=1.2 Hz), 7.90 (d, 1H, J=6.9 Hz), 7.40-7.20 (m, 6H), 7.15 (d, 2H, J=8.6 Hz), 6.85 (d, 2H, J=8.6 Hz), 4.90-4.50 (m, 2H), 4.40-4.00 (m, 7H), 3.30-2.85 (m, 4H), 2.62 (s, 3H), 2.50-1.45 (m, 12H).

### Example 36

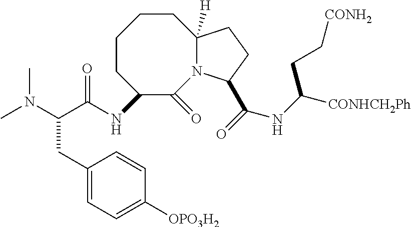

Analytical data: ¹H NMR (300 MHz, CH₃OH-d₄) δ ppm 7.40-7.10 (m, 9H), 4.90-4.70 (m, 1H), 4.50-4.02 (m, 6H), 3.40-3.00 (m, 2H), 2.95 (s, 6H), 2.50-1.40 (s, 16H); ¹³C NMR (75 MHz, CH₃OH-d₄) δ ppm 176.91, 173.08, 172.39, 169.42, 165.52, 138.37, 130.26, 129.56, 128.12, 127.00, 126.79, 120.12, 120.05, 69.21, 61.31, 60.03, 52.91, 49.72, 42.64, 35.93, 34.56, 33.78, 32.03, 31.37, 27.85, 27.23, 25.06, 22.05.

### Example 37

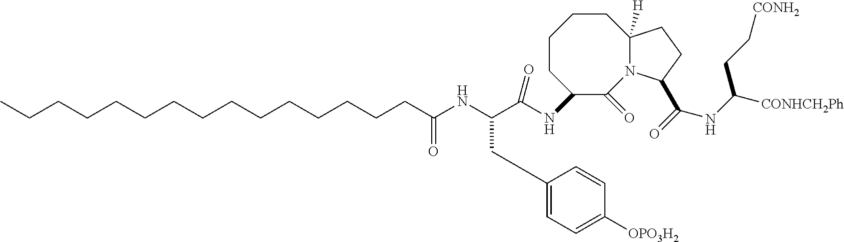

Analytical data: ¹H NMR (300 MHz, CH₃OH-d₄) δ ppm 7.40-7.10 (m, 9H), 4.90-4.75 (m, 1H), 4.70-4.25 (m, 6H), 3.20-2.78 (m, 2H), 2.50-1.20 (m, 46H), 0.90 (t, 3H, J=7.6 Hz).

### Example 38

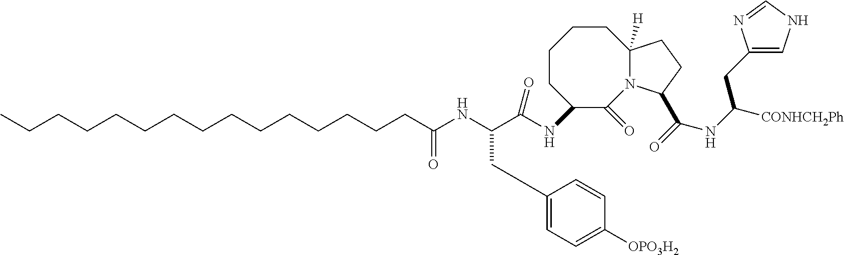

Analytical data: ¹H NMR (300 MHz, CH₃OH-d₄) δ ppm 8.70 (s, 1H), 7.60 (d, 1H, J=6.9 Hz, 7.40-7.02 (m, 10H), 4.70-4.20 (m, 7H), 3.40-2.85 (m, 4H), 2.40-1.20 (m, 42H), 0.90 (t, 3H, J=7.6 Hz).

### Example 39

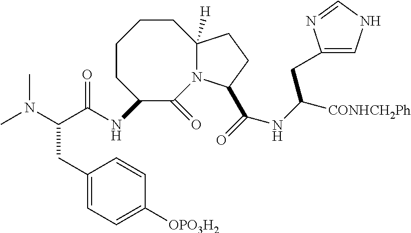

Analytical data: ¹H NMR (300 MHz, CH₃OH-d₄) δ ppm 8.65 (s, 1H), 8.02 (d, 1H, J=7.5 Hz), 7.40-7.10 (m, 10H), 4.80-4.30 (m, 5H), 4.18-4.10 (m, 2H), 3.40-3.20 (m, 4H), 2.98 (s, 6H), 2.50-1.45 (m, 12H); ¹³C NMR (75 MHz, CH₃OH-d₄) δ ppm 173.19, 170.57, 169.15, 165.61, 165.53, 138.33, 133.44, 130.25, 129.62, 129.02, 128.15, 127.05, 126.86, 119.76, 119.69, 117.03, 69.22, 61.41, 59.97, 53.16, 49.48, 41.02, 35.93, 34.77, 33.88, 32.03, 27.25, 26.08, 24.97, 21.92.

### Example 40

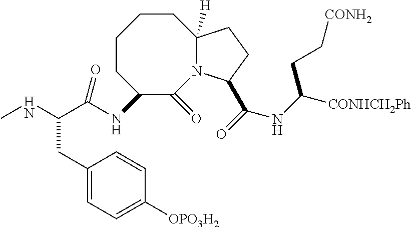

Analytical data: ¹H NMR (300 MHz, CH₃OH-d₄) δ ppm 7.40-7.10 (m, 9H), 4.90-4.70 (m, 1H), 4.50-4.02 (m, 6H), 3.25-3.10 (m, 2H), 2.67 (d, 3H), 2.50-1.50 (m, 16H); ¹³C NMR (75 MHz, CH₃OH-d₄) δ ppm 176.79, 172.99, 172.30, 170.14, 166.21, 138.37, 130.41, 129.51, 128.12, 127.03, 126.80, 120.31, 120.25, 117.45, 62.31, 61.47, 60.09, 52.86, 49.93, 42.63, 35.94, 35.62, 34.22, 32.11, 31.29, 31.22, 27.86, 27.72, 25.51, 21.94.

### Example 41

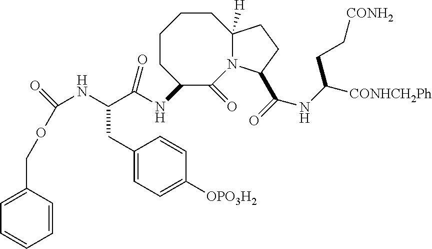

Analytical data: ¹H NMR (CH₃OH-d₄, 300 MHz, 25° C.): 1.31-2.36 (14H, m), 2.73-2.88 (1H, m), 3.15-3.16 (1H, m), 3.88 (1H, br, s), 4.34-4.36 (4H, m), 4.47-4.57 (2H, m), 5.1 (2H, s), 7.11-7.14 (2H, d J=9), 7.22-7.36 (12H, m); ¹³C NMR (CH₃OH-d₄, 75 MHz, 25° C.): 27.7, 27.9, 28.1, 31.0, 31.1, 33.0, 37.2, 43.1, 53.6, 62.3, 66.6, 120.3, 120.3, 127.2, 127.7, 128.0, 128.5, 130.4, 134.0, 137.1, 138.8, 151.0, 157.3, 172.0, 172.1, 173.0, 173.1, 176.8.

### Example 42

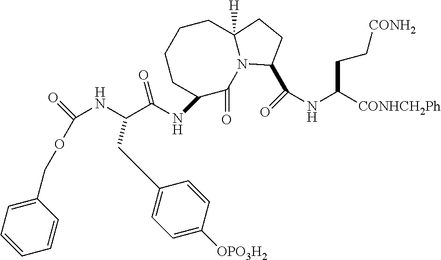

Analytical data: ¹H NMR (CH₃OH-d₄, 300 MHz, 25° C.): 1.31-2.43 (18H, m), 2.78-2.83 (1H, m), 3.02-3.06 (1H, m), 4.24-4.47 (7H, m), 5.1 (2H, s), 7.10-7.34 (14H, m); ¹³C NMR (CH₃OH-d₄, 75 MHz, 25° C.): 20.19, 26.8, 28.1, 28.3, 30.0, 32.0, 35.0, 37.4, 43.1, 53.1, 56.4, 62.3, 63.0, 67.0, 76.2, 77.0, 92.2, 120.1, 120.2, 127.2, 127.5, 127.8, 128.0, 128.5, 128.5, 130.5, 133.5, 137.0, 138.8, 157.2, 172.4, 172.5, 172.7, 173.3, 177.1.

### Example 43

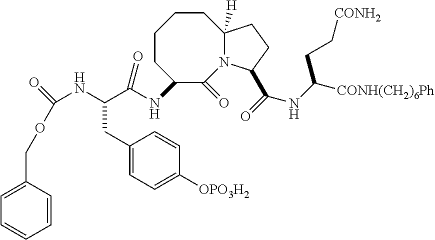

Analytical data: ¹H NMR (CH₃OH-d₄, 300 MHz, 25° C.): δ1.26-1.87 (18H, m), 2.10-2.35 (5H, m), 2.53-2.58 (2H, t J=15 Hz), 2.71-2.77 (1H, m), 3.01-3.10 (1H, m) 3.11-3.15 (2H, t, J=12 Hz) 4.31-4.35 (4H, m), 4.93-4.97 (2H, s), 7.05-7.23 (14H, m); ¹³C NMR (CH₃OH-d₄, 75 MHz, 25° C.): δ20.90, 24.08, 25.28, 26.12, 26.90, 27.43, 27.72, 30.04, 30.17, 34.28, 34.77, 51.51, 58.90, 60.37, 65.05, 118.63, 124.08, 126.16, 126.43, 126.70, 126.83, 126.92, 128.91, 132.16, 135.52, 141.35, 149.26, 155.70, 169.90, 170.87, 171.01, 171.73, 175.53.

### Example 44

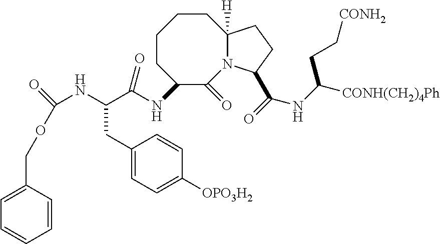

Analytical data: ¹HNMR (CH₃OH-d₄, 300 MHz, 25° C.): δ1.50-2.45 (19H, m), 2.56-2.61 (2H, t J=15 Hz), 2.71-2.82 (1H, dd J=6, 12 Hz), 3.01-3.09 (1H, dd J=6, 15), 3.16-3.21 (2H, t J=15 Hz), 4.24-4.36 (4H, m), 4.93-4.97 (2H, s), 7.03-7.30 (14H, m); ¹³C NMR (CH₃OH-d₄, 75 MHz, 25° C.): δ20.79, 24.09, 26.10, 26.90, 27.27, 27.38, 30.04, 32.86, 33.91, 34.76, 37.59, 48.61, 51.49, 55.00, 58.90, 60.37, 65.05, 118.62, 118.69, 124.19, 126.16, 126.44, 126.75, 126.88, 126.92, 12228.95, 142.32, 135.52, 140.98, 149.11, 150.88, 155.70, 169.93, 170.88, 171.02, 171.73, 175.52, 180.75.

### Example 45

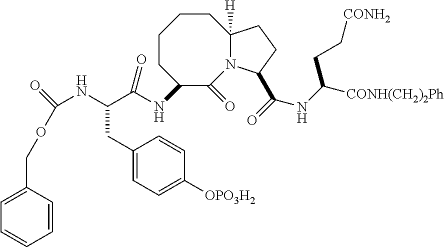

Analytical data: ¹H NMR (CH₃OH-d₄, 300 MHz, 25° C.): δ1.36-2.36 (16H, m), 2.80-2.83 (3H, m), 3.06-3.08 (1H, dd J=4, 15 Hz), 3.40-3.46 (m, 3H), 4.26-4.39 (4H, m), 5.0-5.01 (2H, m), 7.12-7.10 (2H, d, J=6), 7.16-7.35 (12H, m); ¹³C NMR (CH₃OH-d₄, 75 MHz, 25° C.): δ14.47, 20.80, 24.10, 26.07, 26.91, 30.00, 30.92, 32.88, 33.83, 34.78, 35.74, 39.40, 51.45, 55.03, 58.90, 60.35, 65.05, 118.63, 118.69, 124.82, 126.43, 126.97, 127.34, 128.90, 132.12, 135.52, 137.82, 149.12, 155.70, 169.90, 170.89, 171.04, 171.74, 175.50.

### Example 46

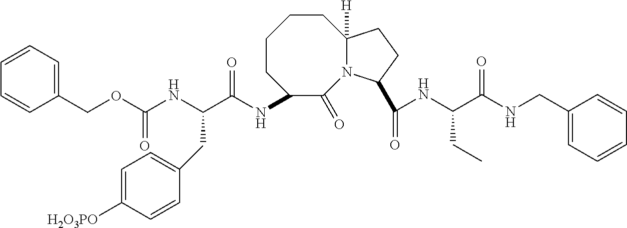

¹H NMR (300 MHz, CD₃OD), δ 7.40-7.24 (m, 10H), 7.22 (d, J=8.5 Hz, 2H), 7.12 (d, J=8.5 Hz, 2H), 5.02 (s, 2H), 4.90-4.80 (m, 1H), 4.55-4.13 (m, 6H), 3.12 (dd, J=4.2, 14.0 Hz, 1H), 2.81 (dd, J=10.0, 14.0 Hz, 1H), 2.30-1.50 (m, 14H), 0.99 (t, J=7.4 Hz, 3H); ¹³C NMR (75 MHz, CD₃OD), δ 172.76, 172.72, 172.03, 171.07, 156.86, 150.23, 150.14, 138.48, 136.69, 133.59, 130.12, 128.09, 127.57, 127.31, 127.11, 126.78, 119.85, 119.78, 66.20, 61.26, 59.97, 56.18, 55.32, 49.80, 42.63, 36.92, 36.00, 34.16, 32.05, 27.05, 25.26, 24.91, 22.10, 9.42.

### Example 47

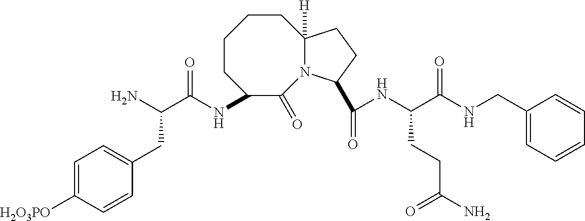

¹H NMR (300 MHz, CD₃OD), δ 7.30-7.15 (m, 9H), 4.85-4.75 (m, 1H), 4.50-4.25 (m, 5H), 4.20-4.05 (m, 1H), 3.17 (dd, J=5.9, 14.0 Hz, 1H), 2.97 (dd, J=8.4, 14.0 Hz), 2.50-1.49 (m, 16H); ¹³C NMR (75 MHz, CD₃OD), δ 178.13, 174.43, 173.72, 171.89, 169.04, 153.24, 139.80, 131.59, 130.98, 129.53, 128.44, 128.21, 121.86, 121.80, 62.91, 61.54, 55.57, 54.31, 51.26, 44.05, 37.90, 37.32, 35.54, 33.54, 32.73, 29.28, 28.67, 26.65, 23.36; Anal. Cacld for: C₃₂H₄₃N₆O₉P.CF₃CO₂H.1.5H₂O: C, 49.33; H, 5.72; N, 10.15. Found: C, 49.38; H, 5.86; 10.18.

### Example 48

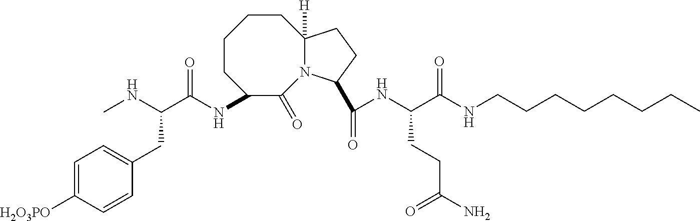

¹H NMR (300 MHz, CD₃OD), δ 7.30-7.10 (m, 4H), 4.90-4.75 (m, 1H), 4.46 (t, J=9.3 Hz, 1H), 4.35-4.20 (m, 2H), 4.05 (t, J=7.1 Hz, 1H), 3.30-3.00 (m, 4H), 2.64 (s, 3H), 2.50-1.49 (m, 18H), 1.48-20 (m, 10H), 0.90 (t, J=6.5 Hz, 3H); ¹³C NMR (75 MHz, CD₃OD), δ 178.22, 174.38, 173.67, 171.39, 167.66, 153.45, 131.59, 130.17, 121.72, 121.66, 63.78, 62.85, 61.46, 54.23, 51.21, 40.43, 37.34, 37.05, 35.71, 33.51, 32.99, 32.79, 32.57, 30.42, 30.41, 30.36, 29.50, 28.71, 27.95, 26.56, 23.72, 23.37, 14.43; Anal. Cacld for: C₃₄H₅₅N₆O₉P.0.5CF₃CO₂H.2H₂O: C, 51.53; H, 7.35; N, 10.30. Found: C, 51.64; H, 7.32; N, 10.23.

### Example 49

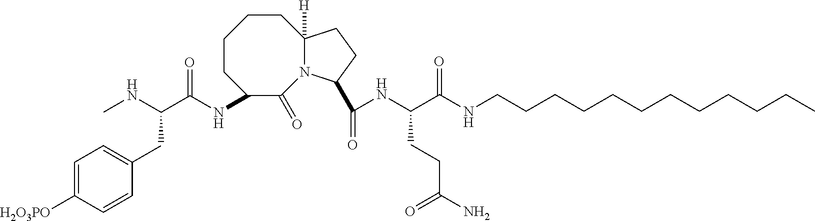

¹H NMR (300 MHz, CD₃OD), δ 7.30-7.10 (m, 4H), 4.90-4.80 (m, 1H), 4.48 (t, J=9.2 Hz, 1H), 4.35-4.20 (m, 2H), 4.07 (t, J=7.2 Hz, 1H), 3.50-3.00 (m, 4H), 2.66 (s, 3H), 2.50-1.48 (m, 16H), 1.47-1.20 (m, 16H), 0.92 (t, J=6.4 Hz, 3H); ¹³C NMR (75 MHz, CD₃OD), δ 176.83, 172.99, 172.29, 170.00, 166.27, 152.06, 151.97, 130.20, 128.80, 120.34, 120.28, 62.39, 61.47, 60.08, 52.85, 49.83, 39.05, 35.97, 35.67, 34.32, 32.14, 31.68, 31.41, 31.19, 29.42, 29.37, 29.09, 28.97, 28.10, 27.33, 26.57, 25.18, 22.35, 21.99, 13.06; Anal. Cacld for: C₃₈H₆₃N₆O₉P.0.5CF₃CO₂H.2H₂O: C, 53.72; H, 7.80; N, 9.64. Found: C, 53.85; H, 7.78; N, 9.52.

### Example 50

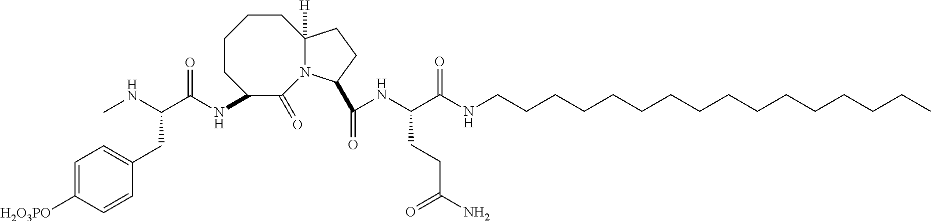

¹H NMR (300 MHz, CD₃OD), δ 7.25-7.08 (m, 4H), 4.88-4.76 (m, 1H), 4.6-4.00 (m, 4H), 3.30-3.01 (m, 4H), 2.70 (s, 3H), 2.50-1.48 (m, 18H), 1.47-1.22 (m, 26H), 0.92 (t, J=6.4 Hz, 3H); ¹³C NMR (75 MHz, CD₃OD), δ 176.82, 172.99, 172.28, 169.96, 166.24, 152.23, 130.09, 128.51, 120.35, 120.28, 62.46, 61.47, 60.05, 52.88, 49.86, 39.04, 35.98, 35.69, 34.37, 32.13, 31.67, 31.44, 31.78, 29.39, 29.35, 29.07, 28.97, 28.10, 27.31, 26.56, 25.18, 22.33, 22.01, 13.03; Anal. Cacld for: C₄₂H₇₁N₆O₉P.0.5CF₃CO₂H.2H₂O: C, 55.65; H, 8.20; N, 9.06. Found: C, 55.81; H, 8.06; N, 8.96.

### Example 51

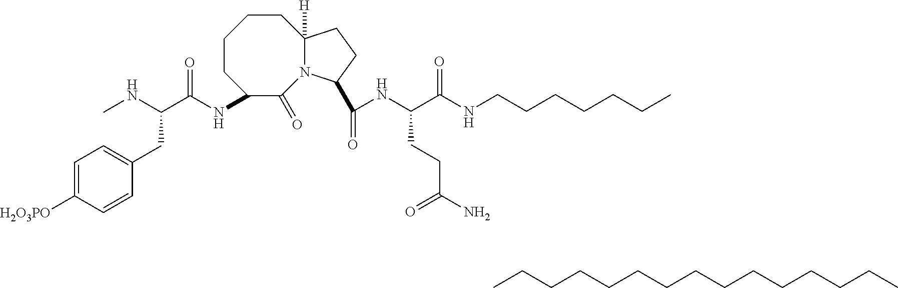

¹H NMR (300 MHz, CD₃OD), δ 7.30-7.04 (m, 4H), 4.90-4.80 (m, 1H), 4.48 (t, J=9.1 Hz, 1H), 4.38-4.20 (m, 2H), 4.03 (t, J=7.2 Hz, 1H), 3.30-3.00 (m, 4H), 2.64 (s, 3H), 2.50-1.20 (m, 52H), 0.90 (t, J=6.5 Hz, 3H); ¹³C NMR (75 MHz, CD₃OD), δ 178.26, 174.48, 173.75, 171.27, 167.69, 153.98, 131.40, 129.57, 121.75, 121.69, 63.90, 62.87, 61.45, 54.37, 51.18, 40.46, 37.40, 37.11, 35.82, 33.55, 33.09, 32.93, 32.55, 30.79, 30.49, 30.39, 29.50, 28.76, 27.99, 26.59, 23.76, 23.43, 14.48; Anal. Cacld for: C₄₆H₇₉N₆O₉P.CF₃CO₂H.2H₂O: C, 55.37; H, 8.13; N, 8.07. Found: C, 55.68; H, 8.27; N, 8.04.

### Example 52

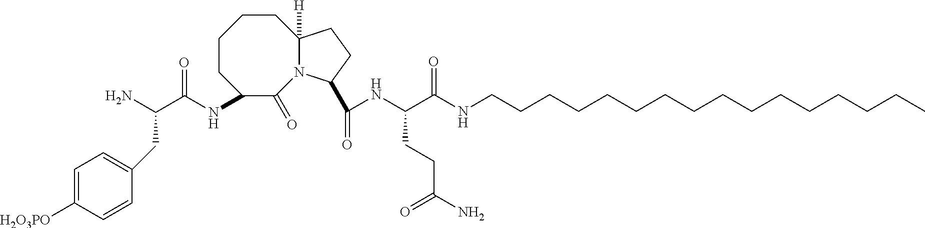

¹H NMR (300 MHz, CD₃OD), δ 7.25-7.06 (m, 4H), 4.88-4.76 (m, 1H), 4.6-4.02 (m, 4H), 3.30-2.80 (m, 4H), 2.50-1.46(m, 18H), 1.47-1.25(m, 26H), 0.92 (t, J=6.4 Hz, 3H).

### Example 53

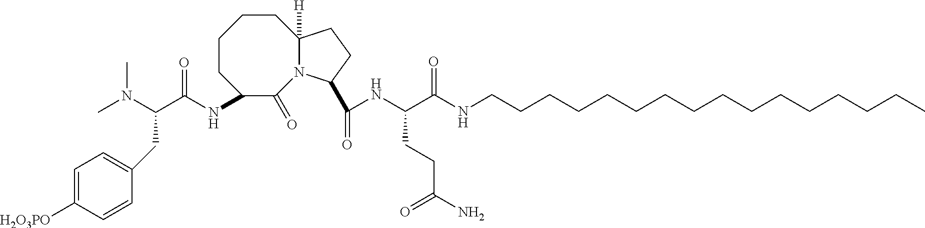

¹H NMR (300 MHz, CD₃OD), δ 7.25-7.06 (m, 4H), 4.78-4.66 (m, 1H), 4.50-4.03 (m, 4H), 3.30-3.04 (m, 4H), 2.97 (s, 6H), 2.50-1.46(m, 18H), 1.47-1.25(m, 26H), 0.92 (t, J=6.4 Hz, 3H).

### Example 54

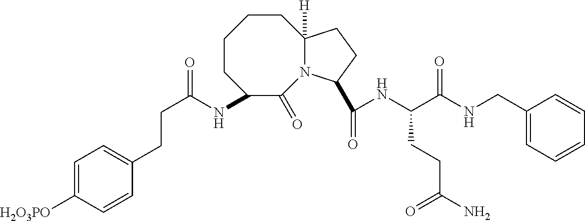

¹H NMR (300 MHz, CD₃OD), δ 7.35-7.20 (m, 5H), 7.19 (d, J=8.6 Hz, 2H), 7.10 (d, J=8.6 Hz, 2H), 4.82-4.70 (m, 1H), 4.45-4.23 (m, 5H), 2.88 (t, J=7.4 Hz, 2H), 2.60-1.42 (m, 18H).

### Example 55

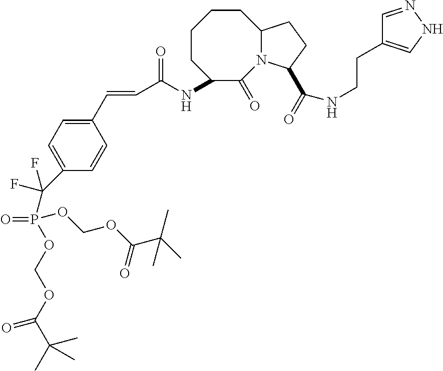

¹H NMR (300 MHz, Acetone-d6), δ 7.82-7.47 (m, 8H), 7.45-7.30 (m, 1H), 6.97 (d, J=15.8 Hz, 1H), 5.01-4.95 (m, 1H), 4.50-4.25 (m, 2H), 3.45-3.28 (m, 2H), 2.70-2.51 (m, 2H), 2.24-1.50 (m, 12H), 1.20 (s, 18H).

### Example 56

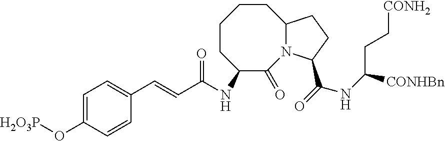

¹H NMR (MeOD-d4, 300 NMR): 7.60-7.40 (m, 3H), 7.30-7.10 (m, 7H), 6.60 (d, J=13 Hz, 1H), 4.50-4.20 (m, 5H), 2.50-1.50 (m, 16H)

MS-ESI: [M+H]⁺=670.22

### Example 57

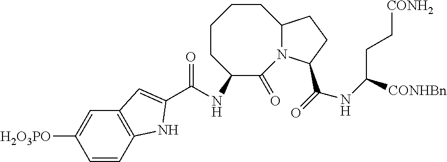

¹H NMR (MeOD-d4, 300 NMR): 8.50-8.35 (m, 2H, N—H), 7.50-7.00 (m, 9H), 5.10-5.00 (m, 1H), 4.50-4.40 (m, 5H), 2.50-1.50 (m, 16H).

MS-ESI: [M+H]⁺=683.20

### Example 58

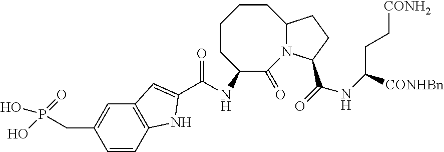

¹H NMR (MeOD-d4, 300 NMR): 7.60-7.40 (m, 2H), 7.30-6.90 (m, 7H), 4.50-4.20 (m, 5H), 3.20 (d, J=21.07 Hz, 2H), 2.50-1.50 (m, 16H).

MS-ESI: [M+H]⁺=681.28

### Example 59

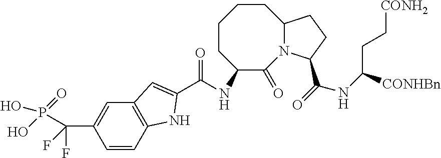

¹H NMR (MeOD-d4, 300 NMR): 7.85 (s, 1H), 7.50-7.40 (m, 2H), 7.30-7.10 (m, 6H), 5.10-5.00 (m, 1H), 4.60-4.40 (m, 5H), 2.50-1.50 (m, 16H)

MS-ESI: [M+H]⁺=717.20

### Example 60

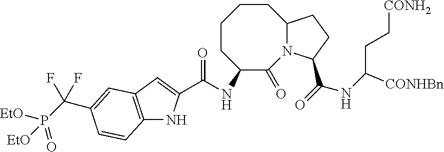

¹H NMR (MeOD-d4, 300 NMR): 7.80 (s, 1H), 7.60-7.50 (m 1H), 7.45-7.35 (m, 1H), 7.20-7.00 (m, 6H), 4.95-4.85 (m, 1H), 4.45-4.20 (m, 5H), 4.15-3.95 (m, 4H), 2.50-1.50 (m, 16H), 1.20-1.00 (, 6H)

MS-ESI: [M+Na]⁺=795.36

### Example 61

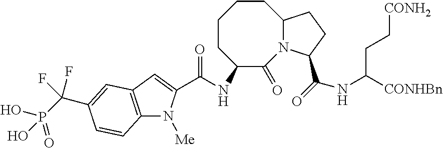

¹H NMR (MeOD-d4, 300 NMR): 7.80 (s, 1H), 7.50 (s, 2H), 7.40-7.05 (m, 5H), 6.98 (s, 1H), 4.40-4.20 (m, 5H), 3.90 (s, 3H), 2.50-1.50 (m, 16H)

MS-ESI: [M+H]⁺=731.21

### Example 62

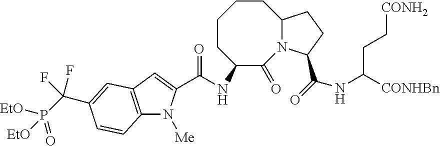

MS-ESI: [M+Na]⁺=809.40

### Example 63

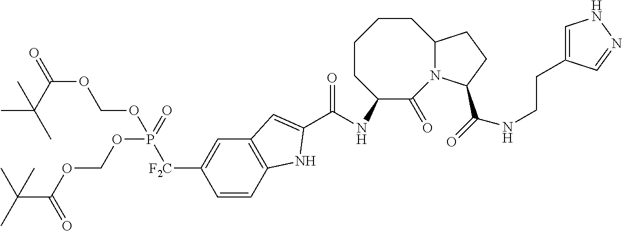

¹H NMR (MeOD-d4, 300 NMR): 8.20-8.10 (m, 1H), 7.80 (s, 1H), 7.60-7.30 (m, 4H), 7.20 (s, 1H), 5.80-5.50 (m, 4H), 5.10-5.00 (m, 1H), 4.45-4.35 (m, 2H), 3.40-3.20 (m, 2H), 2.75-2.65 (m, 2H), 2.40-1.60 (m, 12H), 1.30-1.10 (m, 18H)

MS-ESI: [M+Na]⁺=843.27

### Example 64

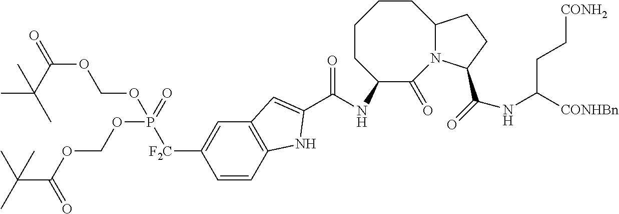

¹H NMR (MeOD-d4, 300 NMR): 8.40-8.20 (m, 1H, N—H), 7.80 (s, 1H), 7.50-7.45 (m, 1H), 7.30-7.20 (m, 1H), 7.25-7.00 (m, 6H), 5.70-5.40 (m, 4H), 5.05-4.90 (m 1H), 4.40-4.10 (m, 6H), 2.50-1.50 (m, 16H), 1.10 (s, 18H)

MS-ESI: [M+Na]⁺=967.40

### Example 65

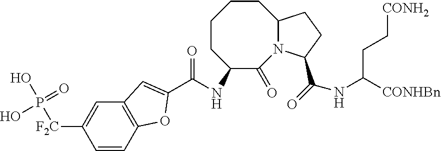

¹H NMR (300 NMR): 8.00 (s, 1H), 7.80-7.60 (m, 2H), 7.60-7.50 (m, 1H), 7.40-7.00 (m, 5H), 5.10-5.00 (m, 1H), 4.80-4.70 (m, 1H), 4.50-4.20 (m, 4H), 2.50-1.50 (m, 16H)

MS-ESI: [M+H]⁺=718.20

### Example 66

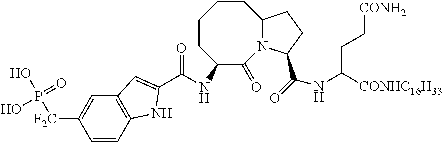

¹H NMR (MeOD-d4, 300 NMR): 7.80 (s, 1H), 7.60-7.30 (m, 2H), 7.00 (s, 1H), 5.10-5.00 (m, 1H), 4.50-4.10 (m, 4H), 3.30-3.10 (m, 2H), 2.50-1.50 (m, 18H), 1.40-1.10 (m, 26H), 0.8 (t, J=6.04 Hz, 3H)

MS-ESI: [M+H]⁺=851.30

### Example 67

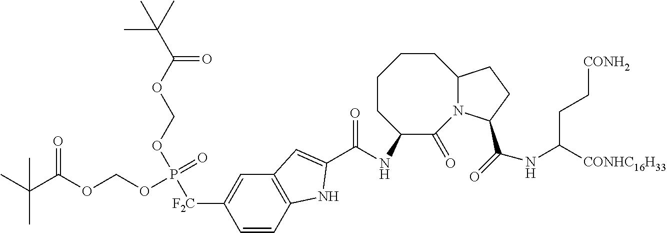

¹H NMR (MeOD-d4, 300 NMR): 7.80 (s, 1H), 7.70-7.30 (m, 2H), 7.20 (s, 1H), 5.80-5.50 (m, 4H), 5.10-5.00 (m, 1H), 4.60-4.40 (m, 4H), 3.20-3.00 (m, 2H), 2.50-1.50 (m, 18H), 1.40-1.10 (m, 26H), 1.20 (s, 18H), 0.8 (t, J=6.04 Hz, 3H)

MS-ESI: [M+Na]⁺: 1101.60

### Example 68

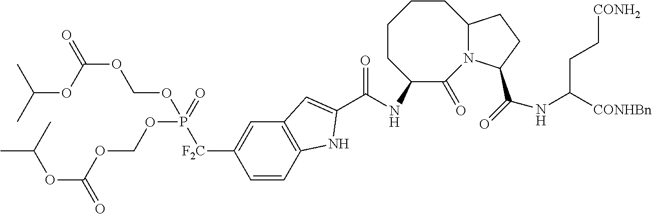

¹H NMR (MeOD-d4, 300 NMR): 7.80 (s, 1H), 7.50-7.40 (m, 1H), 7.30-7.20 (m, 1H), 7.20-7.00 (m, 6H), 5.60-5.40 (m, 4H), 5.00-4.90 (m, 1H), 4.80-4.70 (m, 2H), 4.40-4.10 (m, 6H), 2.50-1.50 (m, 16H), 1.30-1.10 (m, 12H)

MS-ESI: [M+Na]⁺=971.33

### Example 69

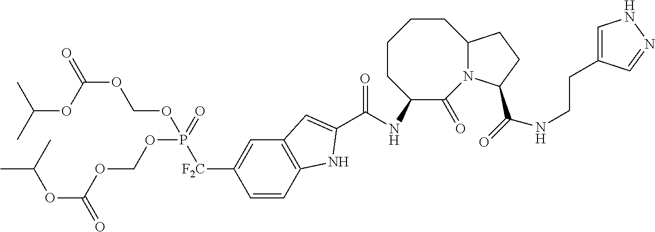

¹H NMR (MeOD-d4, 300 NMR): 7.80 (s, 1H), 7.60-7.20 (m, 4H), 1.2) (s, 1H), 5.60-5.40 (m, 4H), 5.00-4.90 (m, 1H), 4.80-4.70 (m, 2H), 4.40-4.10 (m, 2H), 3.30-3.10 (m, 2H), 2.70-2.50 (m, 2H), 2.20-1.50 (m, 12H), 1.20-1.00 (m, 12H),

MS-ESI: [M+Na]⁺=847.26

### Example 70

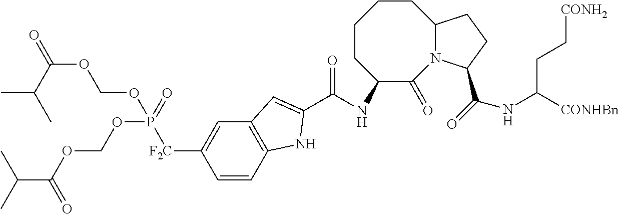

¹H NMR (MeOD-d4, 300 NMR): 7.85 (s, 1H), 7.60-7.50 (m, 1H), 7.40-7.30 (m, 1H), 7.35-7.10 (m, 6H), 5.80-5.60 (m, 4H), 5.10-5.00 (m,)H), 4.60-4.50 (m, 6H), 3.60-1.50 (m, 18H), 1.15-1.05 (m, 12H)

MS-ESI: [M+Na]⁺=939.29

### Example 71

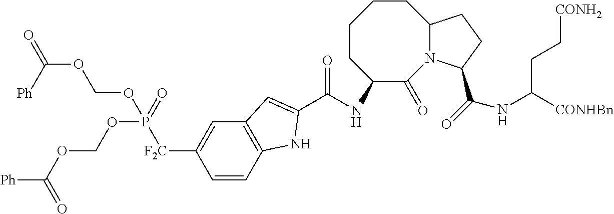

¹H NMR (MeOD-d4, 300 NMR): 7.95-7.80 (m, 4H), 7.80-7.75 (s, 1H), 7.65-7.50 (m, 2H), 7.50-7.20 (m,)1H), 7.10 (s, 1H), 6.00-5.80 (m, 4H), 4.50-4.20 (m, 6H), 2.50-1.50 (m, 16H)

MS-ESI: [M+Na]⁺=1007.28

### Example 72

### In Vitro Competitive STAT3 Binding Assay

To quantitatively determine the binding affinities of STAT3 inhibitors to STAT3 protein, a sensitive and quantitative in vitro binding assay was developed based upon fluorescence polarization (FP) method. For this FP assay, 5-carboxyfluorescein (5-Fam) was coupled to a phosphopeptide (phospho-Tyr-Leu-Pro-Gln-Thr-Val-amide) with two beta-alanine and one glycine residues as the spacer between 5-Fam and the phosphopeptide to obtain 5-Fam-beta-Ala-beta-Ala-Gly-phospho-Tyr-Leu-Pro-Gln-Thr-Val-amide as the fluorescently tagged tracer, which was named as STT2 peptide. The K_{d} value of the STT2 peptide to STAT3 protein was determined to be 47 nM in saturation experiments. The recombinant human STAT3 protein (residues 122-722) fused to His-tag was stable and soluble.

Competitive binding experiments were carried out using 150 nM of STAT3 protein and 5 nM of STT2 peptide with different concentrations of a tested STAT3 inhibitor. For each experiment, two controls were included. One control contained STAT3 protein and STT2 peptide, and the second control contained STT2 peptide only. The polarization values were measured after 2-3 hours of incubation using an ULTRA READER (Tecan U.S. Inc., Research Triangle Park, N.C.). IC₅₀ values, the inhibitor concentration at which 50% of the fluorescently tagged STT2 peptide is displaced by STAT3 inhibitors, was determined from a plot using nonlinear least-squares analysis using GRAPHPAD PRISM software (GraphPad Software, Inc., San Diego, Calif.).

FP experiments were performed in 96-well, black round-bottom plates (Microfluor 2, Fisher Scientific) using the Ultra plate reader (Tecan). To each well, 5 nM of fluorescein-labeled probe (GO300-FL) and 50 nM of recombinant STAT3 (127-722 amino acid) protein were added to a final volume of 125 μl in the assay buffer (50 mM NaCl, 10 mM Hepes pH 7.5, 1 mM EDTA pH 8.0, 0.1% Nonidet, 2 mM DTT). The plate was mixed on a shaker for 15 min and incubated at room temperature for 3 h to reach equilibrium. The polarization values in millipolarization (mP) units were measured at an excitation wavelength of 485 nm and an emission wavelength of 530 nm. All experimental data were analyzed using Prism 3.0 software (GraphPad Software), and the inhibition constants were determined by nonlinear curve fitting as the concentration of the STAT3 at which 50% of the ligand is bound.

### Example 73

### Cell Growth Assay

The effect of STAT3 compounds on cell growth was evaluated by a WST-8 [2-(2-methoxy-4-nitrophenyl)-3-(4-nitrophenyl)-5-(2,4-disulfophenyl)-2H-tetrazolium, monosodium salt assay (Dojindo Molecular Technologies, Inc). Cells (3000-4000 cells in each well) were cultured in 96-well tissue culture plates in medium (200 μL) containing various concentrations of STAT3 compounds for 4 days. At the end of incubation, WST-8 dye (20 μL) was added to each well and incubated for an additional 1-3 h, and then the absorbance was measured in a microplate reader (Molecular Devices) at 450 nm. Cell growth inhibition was evaluated as the ratio of the absorbance of the sample to that of the control.

TABLE 1
Summary of binding affinities to STAT3 of designed compounds and their cellular
activity in inhibition of cell growth in cancer cell lines with high levels of activated STAT3.

| | | | Inhibition of | |
|---|---|---|---|---|
| | | | cell growth | |
| | | Binding | IC₅₀ [μM] | |
| | | affinities | MDA-MB- | MDA-MB- |
| | | to STAT3 | 231 cancer | 468 cancer |
| Example | Structure | IC₅₀ [μM] | cell line | cell line |
| | 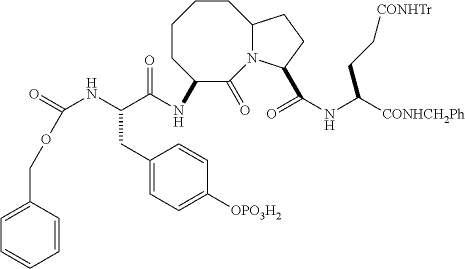 | <20 | <50 | <50 |
| 41 | 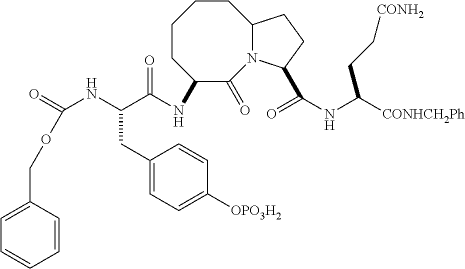 | <1 | >50 | >100 |
| | 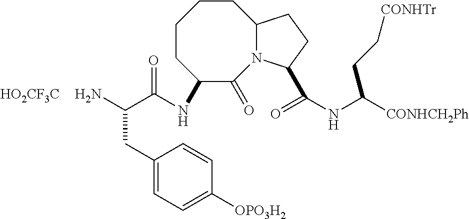 | <10 | <10 | <10 |
| | 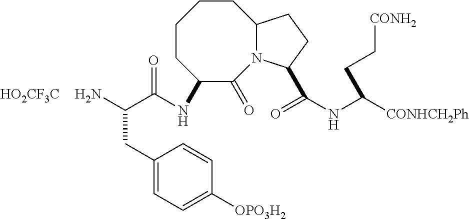 | <1 | >20 | >100 |
| | 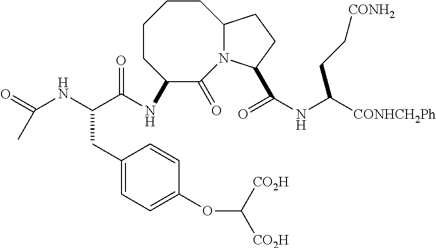 | <10 | >50 | >100 |
| 1 | 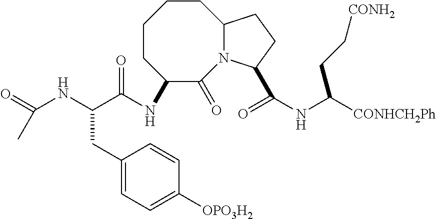 | <1 | >20 | >100 |
| | 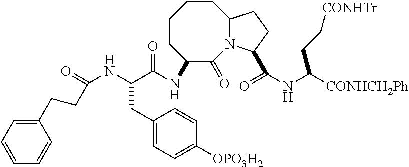 | <20 | >100 | >50 |
| 2 | 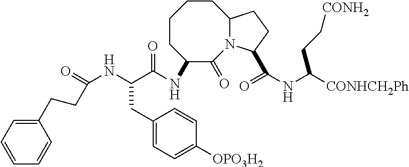 | <1 | >20 | >100 |
| 3 | 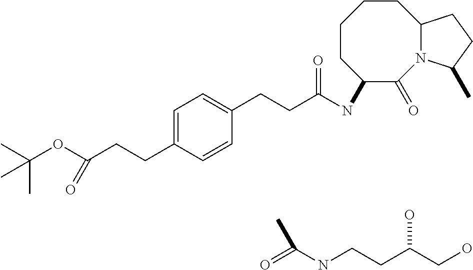 | >100 | >30 | >100 |
| 4 | 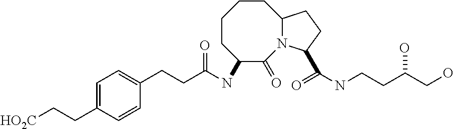 | >100 | >30 | >100 |
| 5 | 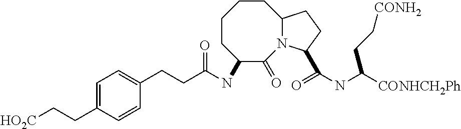 | >100 | >50 | >100 |
| 6 | 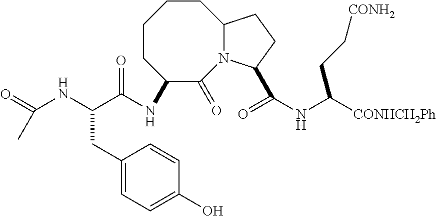 | >100 | >100 | >100 |
| | 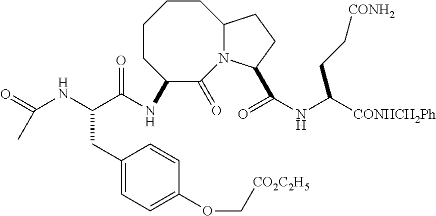 | >100 | >100 | >100 |
| | 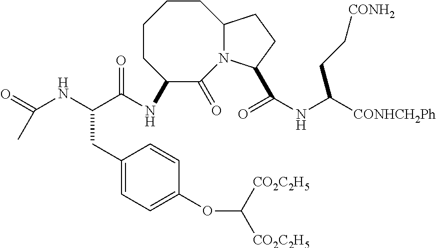 | >100 | >30 | >100 |
| 7 | 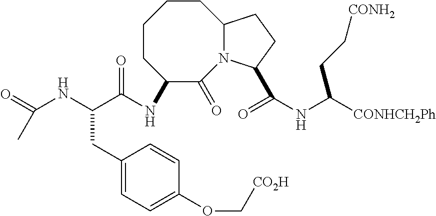 | >50 | >100 | >100 |
| 8 | 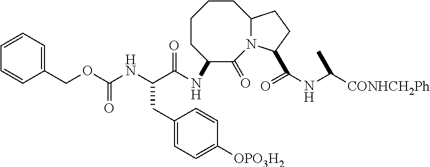 | >20 | >10 | >10 |
| 9 | 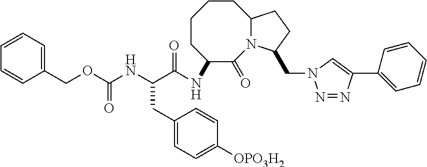 | >20 | >5 | >5 |
| 10 | 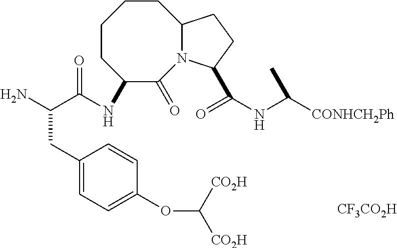 | >100 | >50 | >100 |
| | 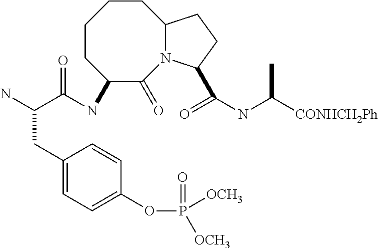 | >100 | >50 | >100 |
| 11 | 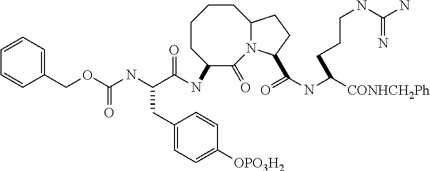 | >5 | >50 | >100 |
| 12 | 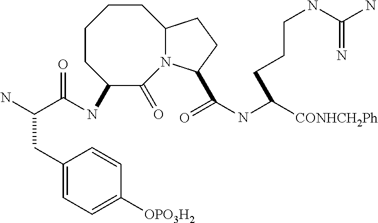 | >5 | <50 | >100 |
| 13 | 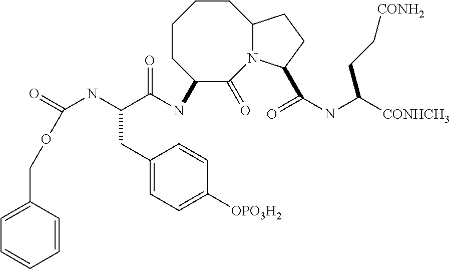 | <1 | >100 | >100 |
| 14 | 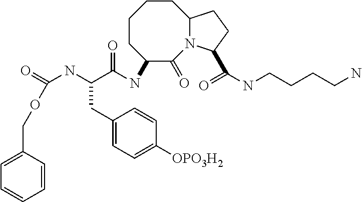 | <10 | >100 | >100 |
| | 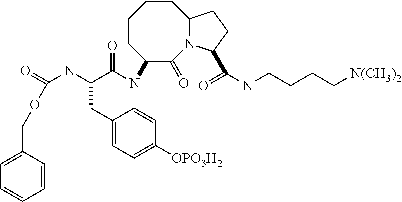 | <30 | >100 | >100 |
| 15 | 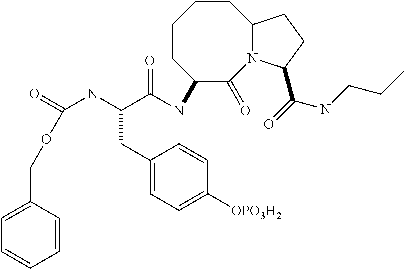 | <30 | >30 | >100 |
| 16 | 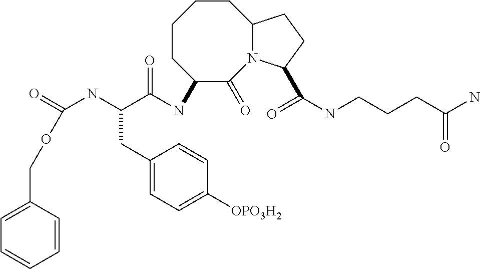 | <30 | >100 | >100 |
| 17 | 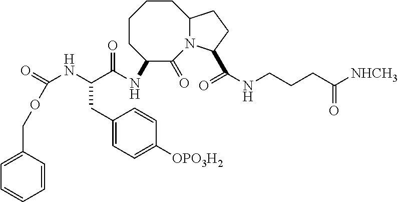 | <30 | >100 | >100 |
| 18 | 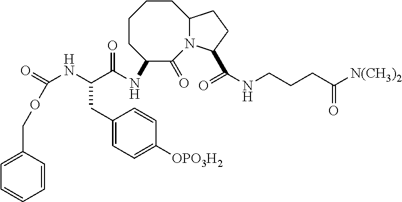 | <30 | >100 | >100 |
| 19 | 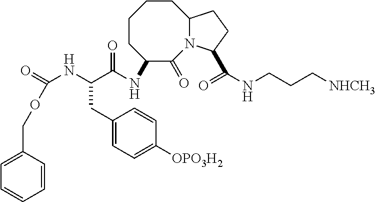 | <100 | >100 | >100 |
| 20 | 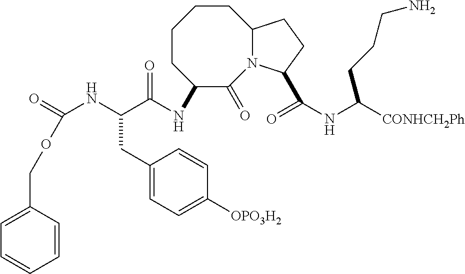 | <10 | >50 | >100 |
| 21 | 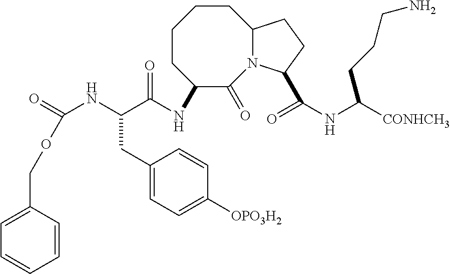 | <10 | >100 | >100 |
| 22 | 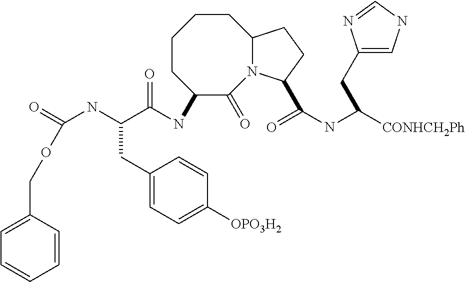 | <10 | >50 | >100 |
| 23 | 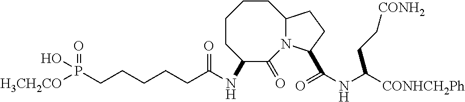 | >100 | >100 | NT |
| 24 | 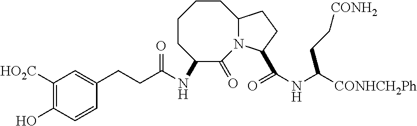 | >100 | >100 | >100 |
| 25 | 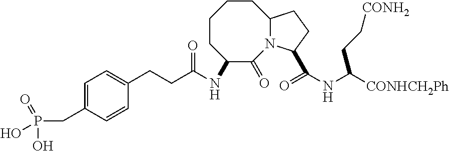 | >100 | >100 | >100 |
| | 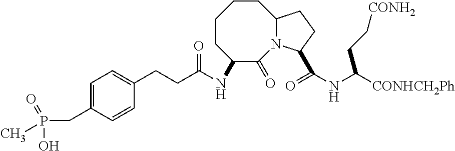 | >100 | >100 | >100 |
| | 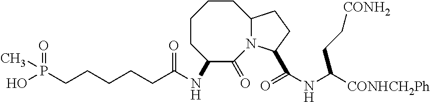 | >100 | >100 | >100 |
| | 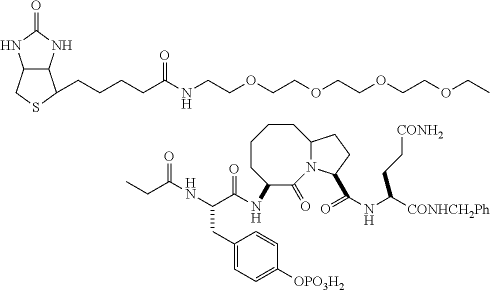 | <1 | NT | NT |
| | 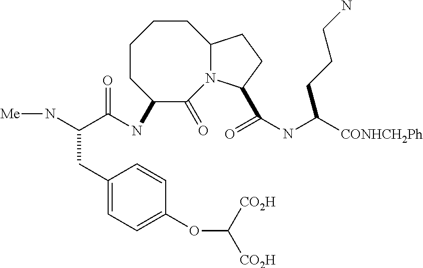 | >10 | >100 | >100 |
| | 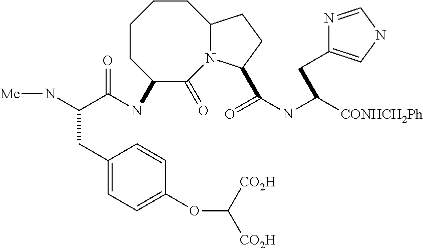 | >10 | >100 | >100 |
| | 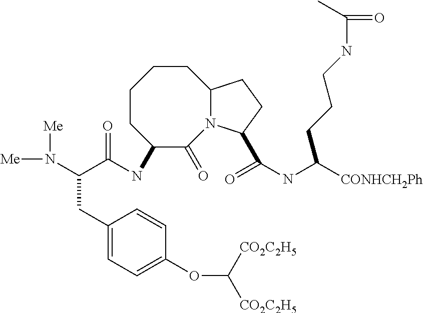 | >10 | >100 | >100 |
| | 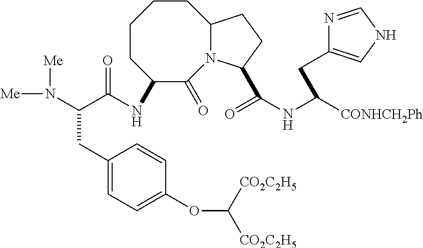 | >10 | >100 | >100 |
| | 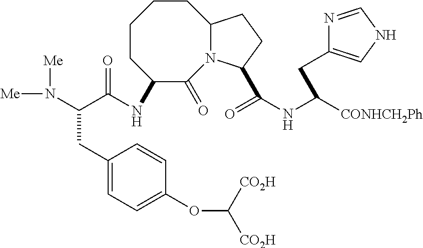 | >10 | >100 | >100 |
| | 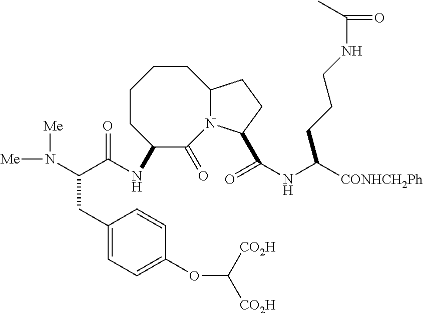 | >10 | >100 | >100 |
| 26 | 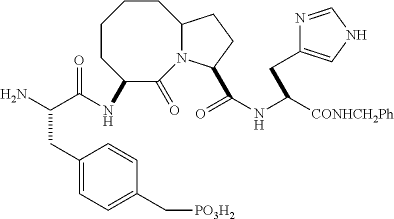 | >10 | >100 | >100 |
| 27 | 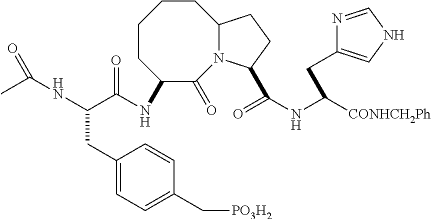 | >10 | >100 | NT |
| 28 | 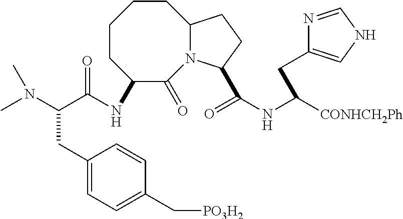 | >10 | NT | >100 |
| 29 | 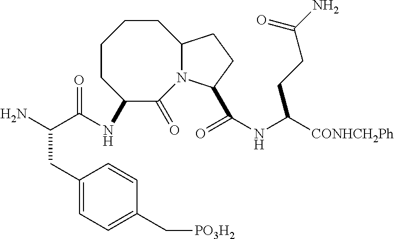 | <30 | >100 | >100 |
| 30 | 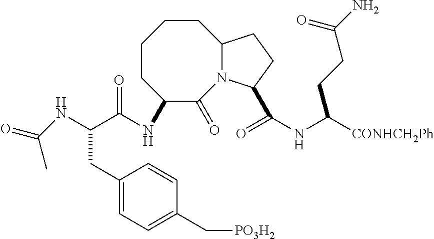 | <30 | >100 | >100 |
| 31 | 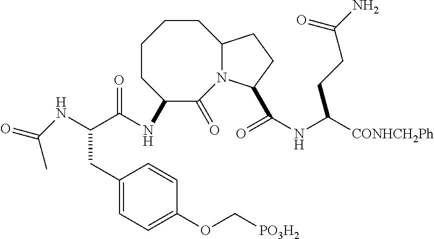 | <10 | >100 | NT |
| | 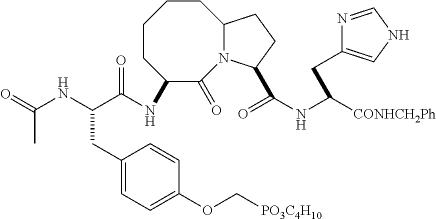 | >100 | >100 | >100 |
| | 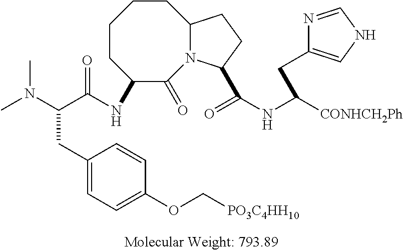 | >100 | >100 | >100 |
| 32 | 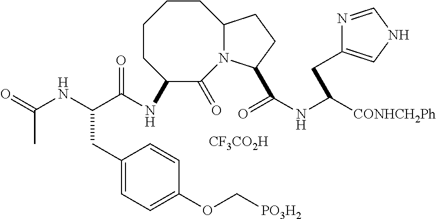 | <30 | >100 | >100 |
| 33 | 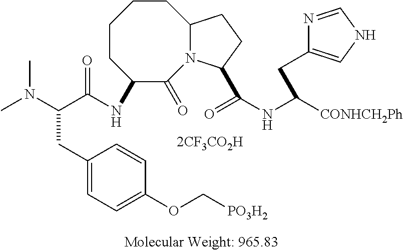 | >100 | >100 | >100 |
| 34 | 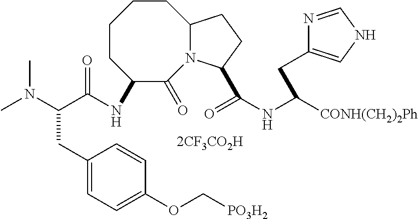 | <30 | >100 | >100 |
| 35 | 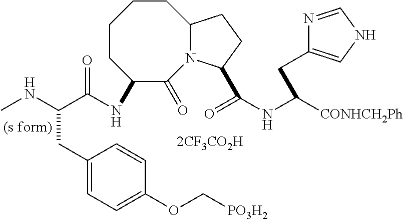 | <100 | >100 | >100 |
| | 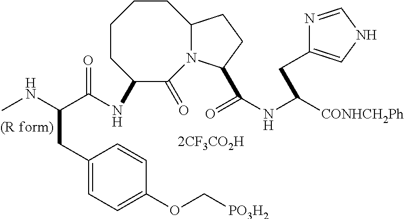 | <100 | >100 | >100 |
| | 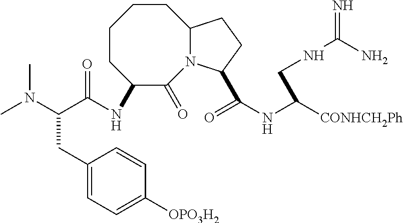 | <100 | >50 | >100 |
| | 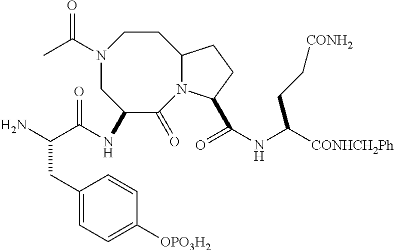 | <5 | >50 | >100 |
| | 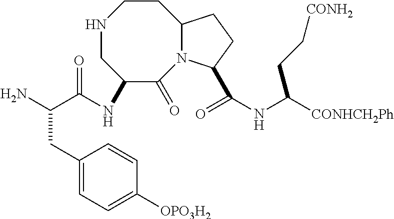 | <100 | >100 | >100 |
| 36 | 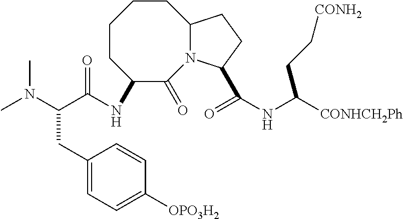 | <5 | >100 | >100 |
| 37 | 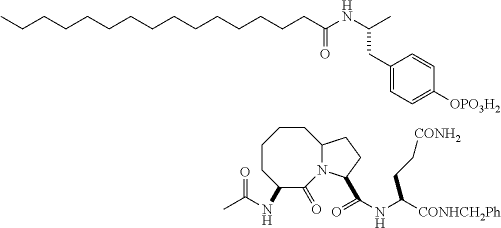 | <10 | <50 | <50 |
| 38 | 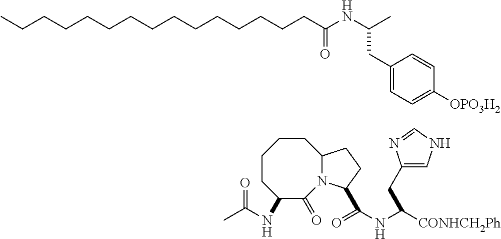 | <100 | <50 | <60 |
| 39 | 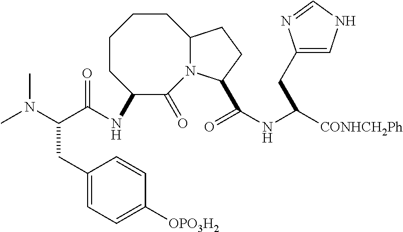 | <10 | >100 | >100 |
| 40 | 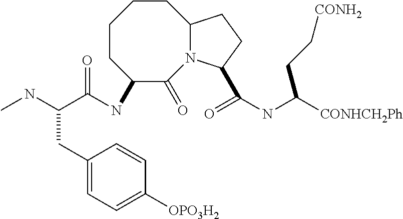 | <1 | >30 | >100 |
| | 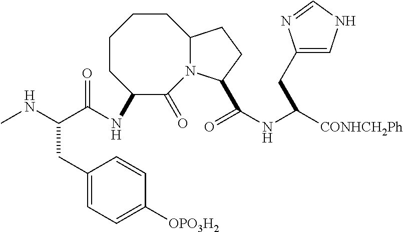 | <30 | >50 | >100 |
| | 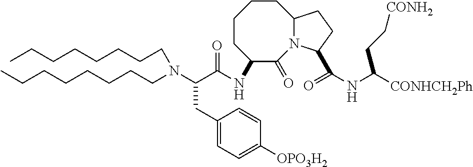 | <100 | >100 | NT |
| | 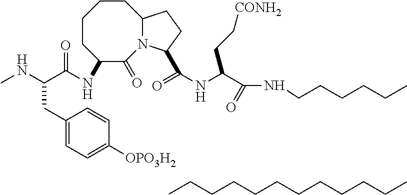 | <1 | <20 | <20 |
| | 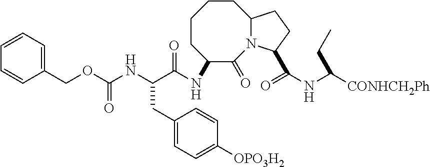 | >10 | >30 | >30 |
| | 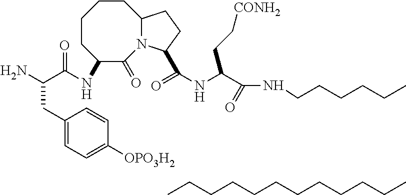 | >5 | <50 | <50 |
| | 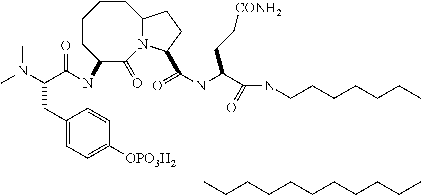 | <5 | <20 | <20 |
| | 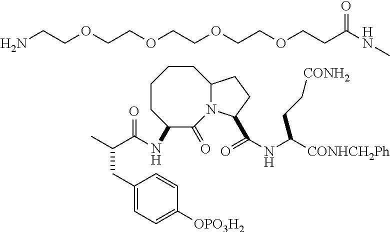 | <5 | >100 | >100 |
| | 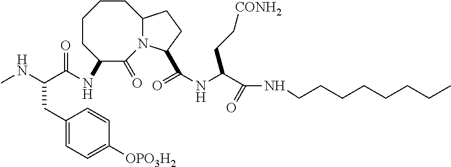 | <5 | >100 | >100 |
| | 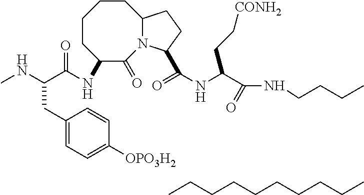 | <5 | <30 | <20 |
| | 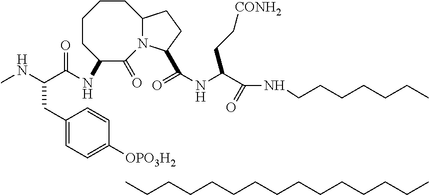 | <5 | <30 | <30 |
| | 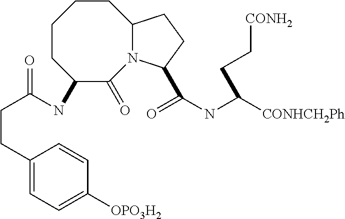 | <5 | >100 | >100 |
| | 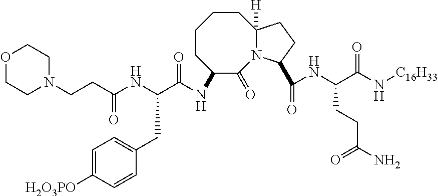 | <5 | NT <40 | NT <40 |
| | 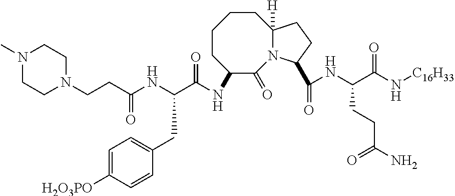 | <5 | >20 | >20 |
| | 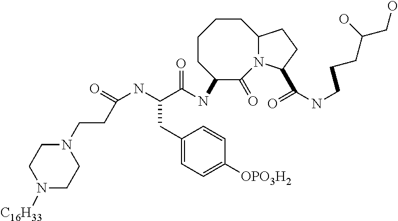 | <10 | <20 | <20 |
| | 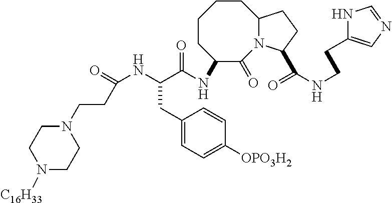 | <10 | <10 | <10 |
| | 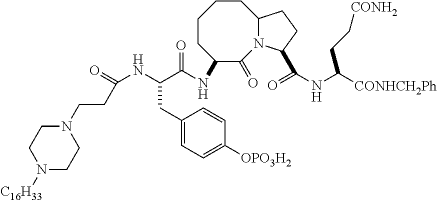 | <1 | <10 | <10 |
| | 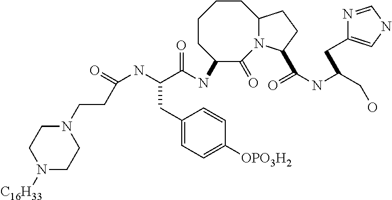 | <5 | <20 | <20 |
| | 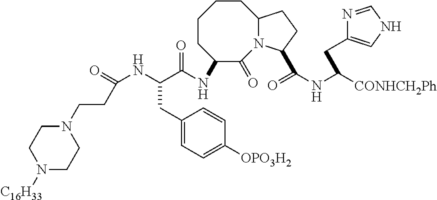 | <5 | <30 | <30 |
| | 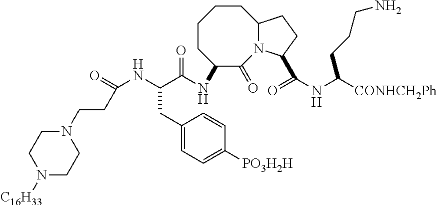 | <5 | <50 | <50 |
| | 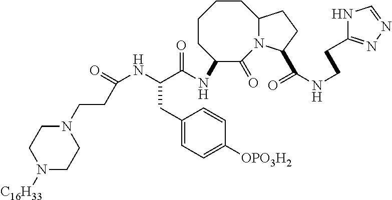 | <5 | <20 | <20 |
| | 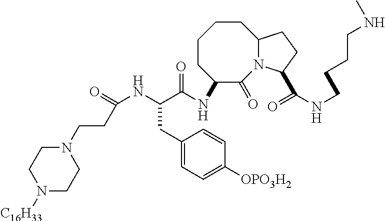 | <10 | <50 | <50 |
| | 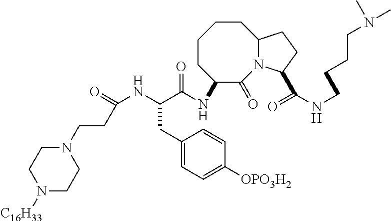 | <20 | <100 | <50 |
| | 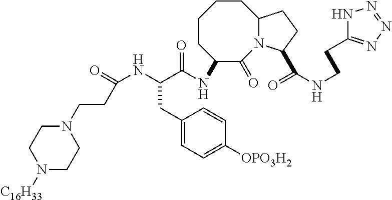 | NT | <50 | <50 |
| | 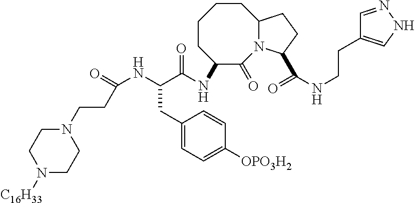 | <5 | <20 | <20 |
| | 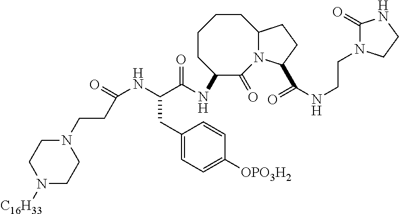 | <5 | <30 | <20 |
| | 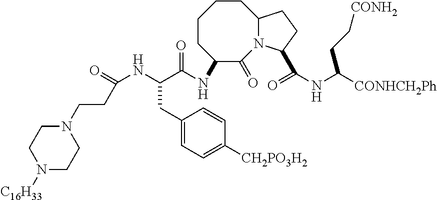 | >100 | >100 | >100 |
| | 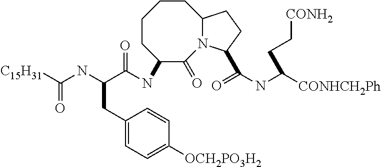 | <5 | <100 | <50 |
| 56 | 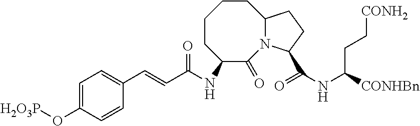 | <1 | >100 | >100 |
| 57 | 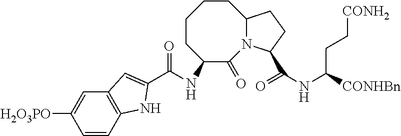 | <1 | >100 | >100 |
| 58 | 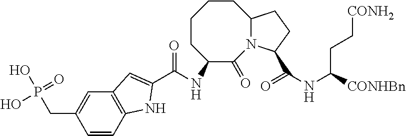 | <10 | >100 | >100 |
| 59 | 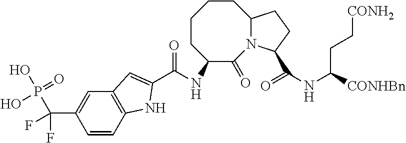 | <1 | NT | >100 |
| 61 | 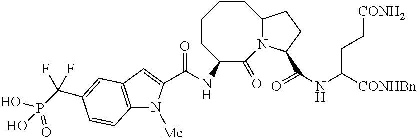 | <5 | NT | >100 |
| 65 | 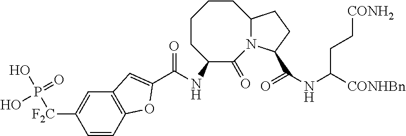 | >10 | NT | >100 |
| 66 | 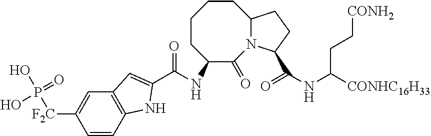 | <1 | NT | >100 |

NT, not tested

TABLE 2
Summary of binding affinities to STAT3 of designed pro-drugs and their cellular
activity in inhibition of cell growth in cancer cell lines with high levels of activated STAT3.

| | | | | |
|---|---|---|---|---|
| | 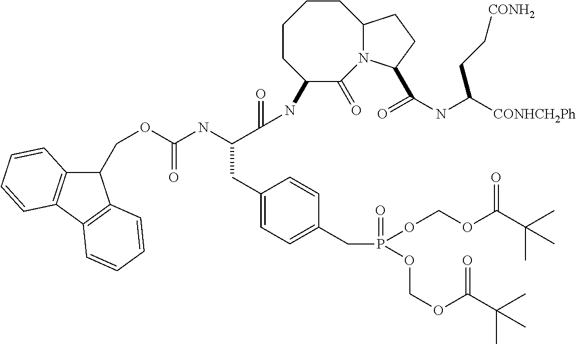 | NT | <10 | <10 |
| | 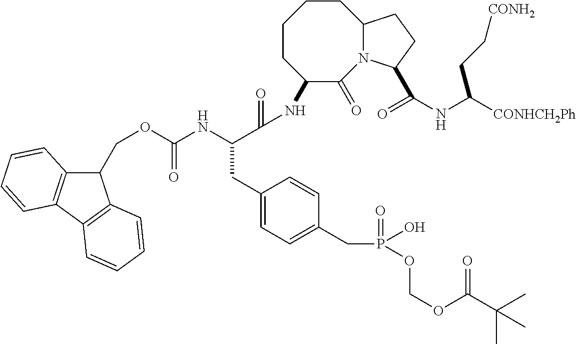 | NT | NT | NT |
| | 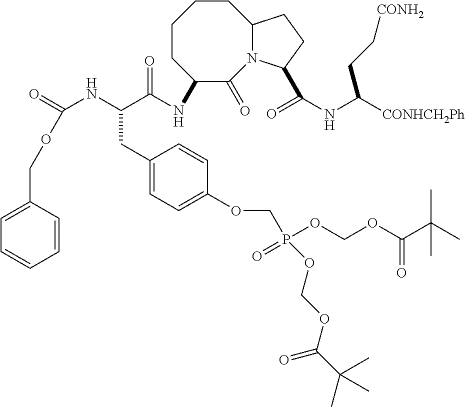 | NT | >50 | >50 |
| | 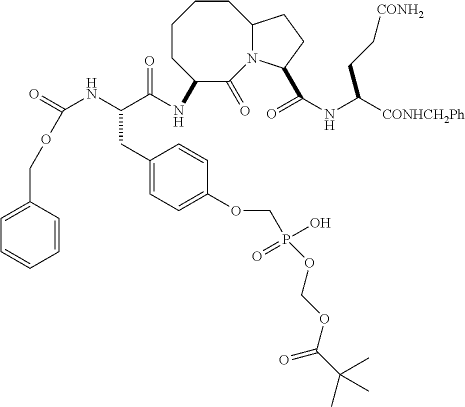 | NT | >50 | >50 |
| | 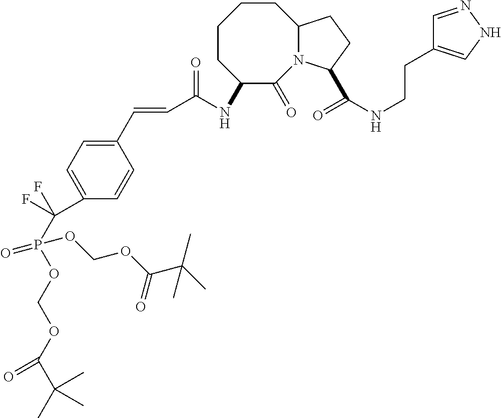 | NT | NT | <30 |
| | 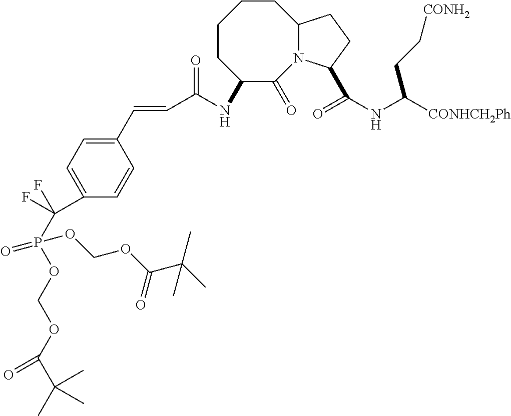 | NT | NT | >100 |
| | 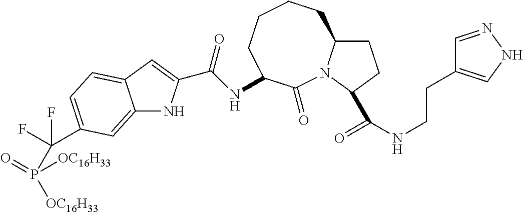 | NT | NT | >100 |
| | 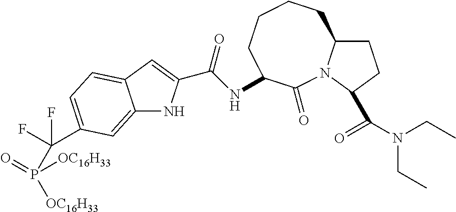 | NT | NT | >100 |
| | 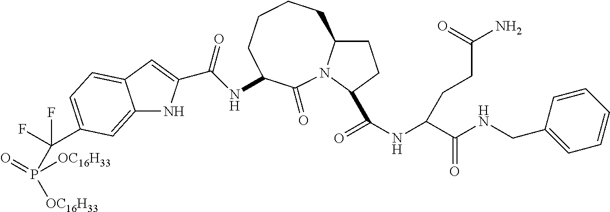 | NT | NT | <30 |
| | 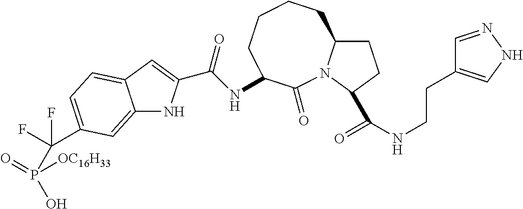 | NT | NT | >100 |
| | 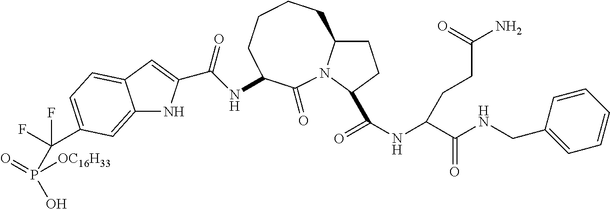 | NT | NT | >50 |
| | 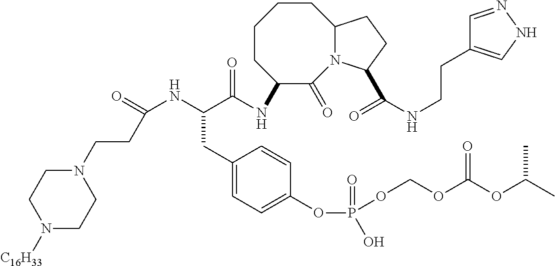 | NT | NT | NT |
| 62 | 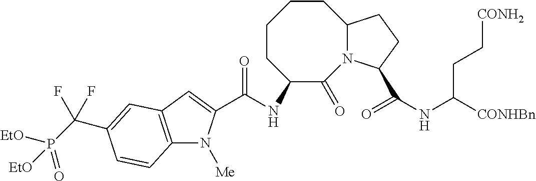 | NT | NT | >100 |
| 63 | 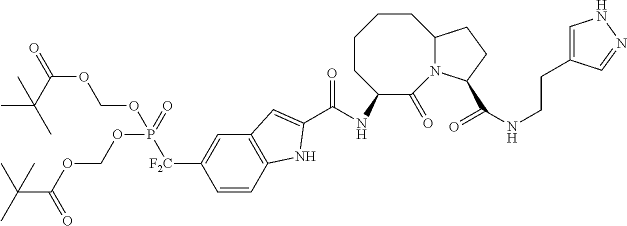 | NT | NT | <50 |
| 64 | 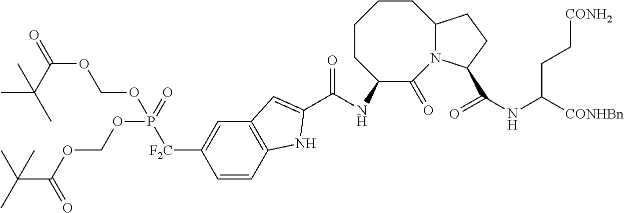 | NT | NT | >100 |
| 67 | 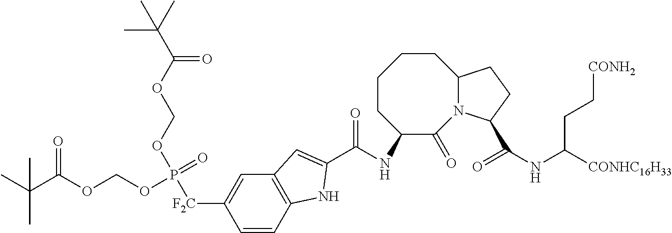 | NT | NT | >100 |
| 68 | 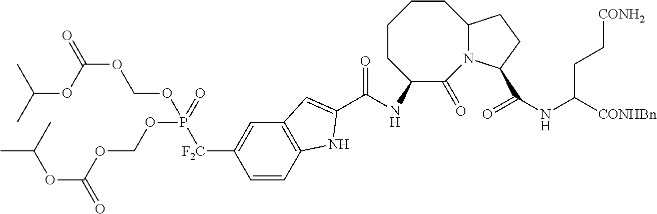 | NT | NT | >100 |
| 69 | 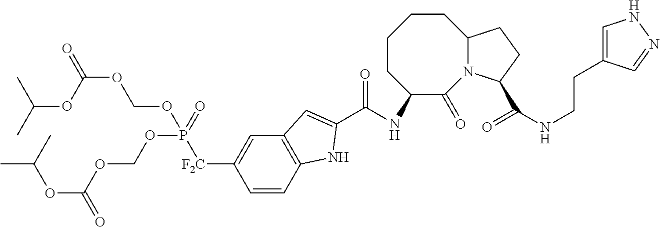 | NT | NT | >100 |
| 70 | 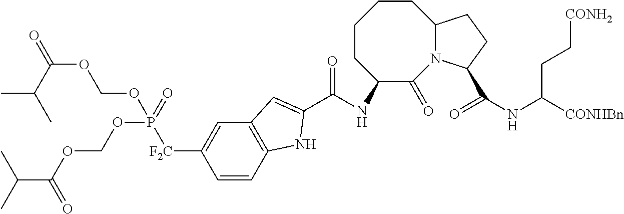 | NT | NT | NT |
| | 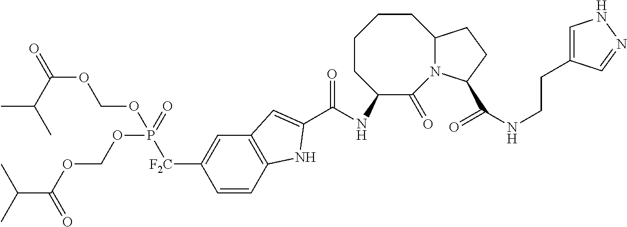 | NT | NT | NT |
| 71 | 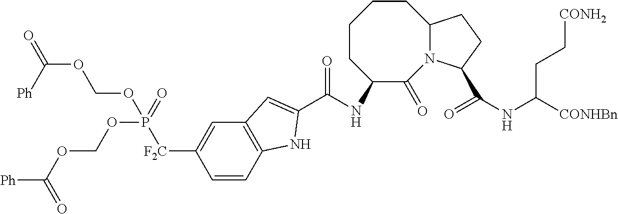 | NT | NT | NT |
| | 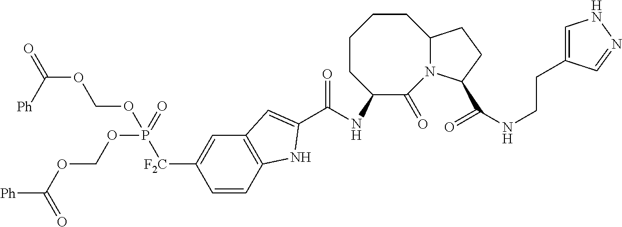 | NT | NT | NT |

NT, not tested

Compounds having an affinity for STAT3 have been disclosed previously. Some of these prior compounds possess moderate affinities and inhibitory effects on STAT3, and therefore have been proposed as suitable for treating diseases such as a cancer. Unfortunately, these compounds have not proved satisfactory. Consequently, an ongoing need exists to provide new compounds that preferably have a high affinity for STAT3 and a good pharmacological profile, e.g., a high bioavailability and good metabolic stability. The present STAT3 inhibitors have been designed to meet these beneficial properties.

In summary, a series of compounds of structural formula (I) and (II) have been designed, synthesized, and evaluated for their binding to STAT3. The present invention therefore identifies potent STAT3 inhibitors, that have a therapeutic potential for the treatment of cancers, for example, and other conditions in which inhibition of STAT3 is desirable.

The present invention also can be applied to cell populations ex vivo. For example, the present STAT3 inhibitor can be used ex vivo to determine the optimal schedule and/or dosing of administration of a present STAT3 inhibitor for a given indication, cell type, patient, and other parameter. Information gleaned from such use can be used for experimental purposes or in the clinic to set protocol for in vivo treatment. Other ex vivo uses for which the invention is suited are apparent to those skilled in the art.

### Appendix A

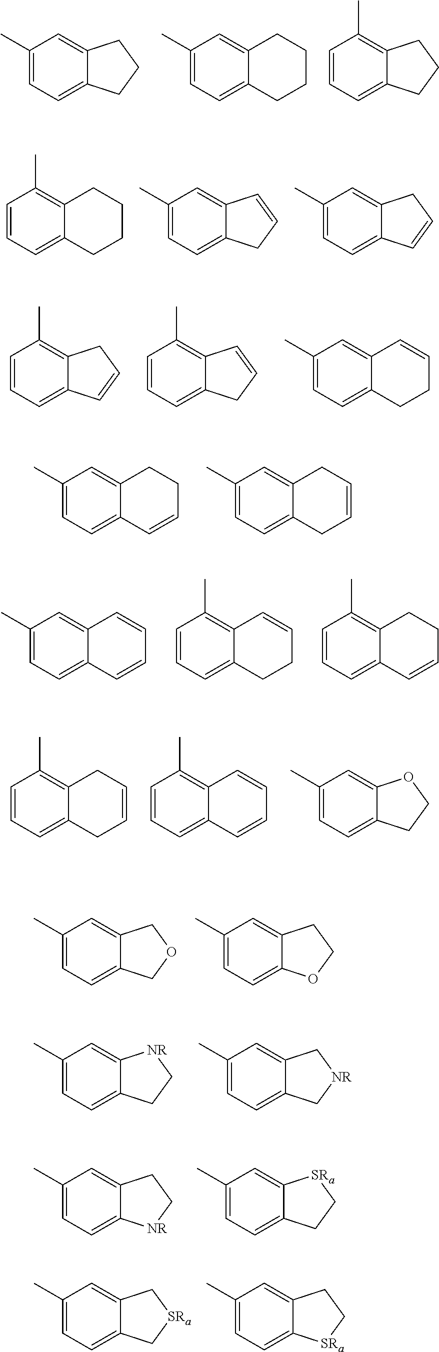

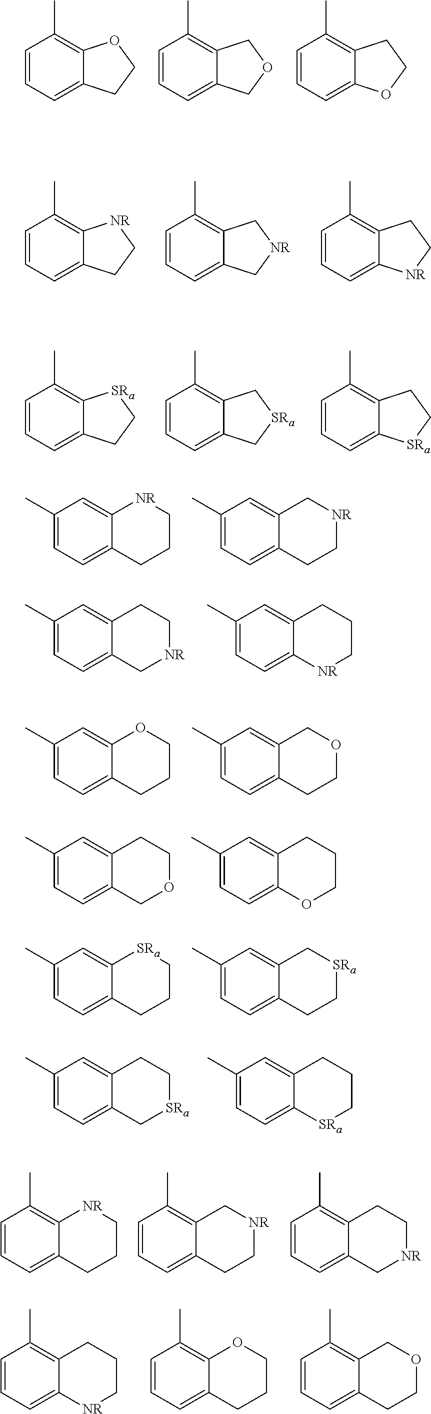

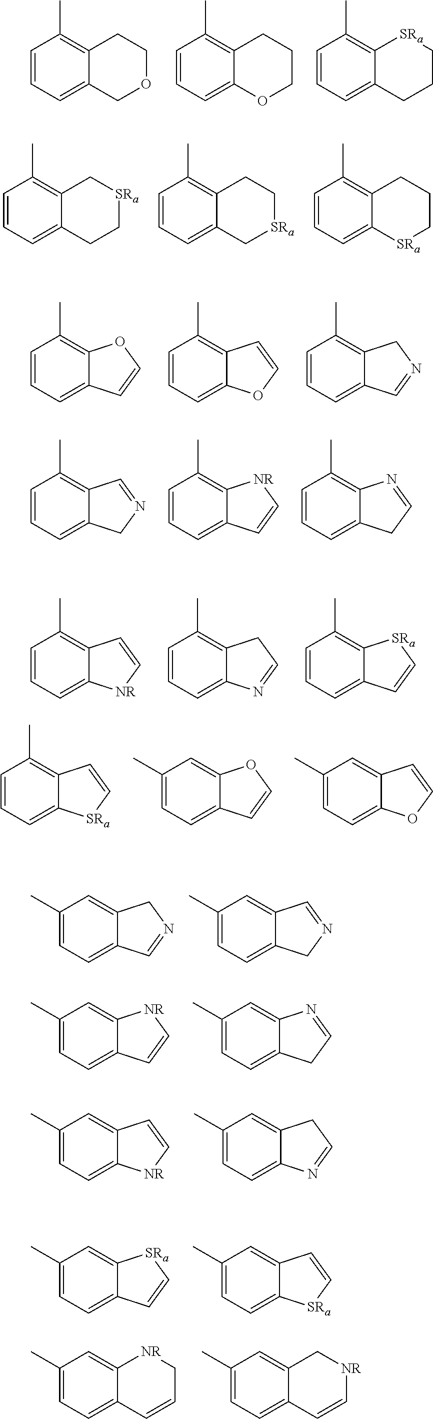

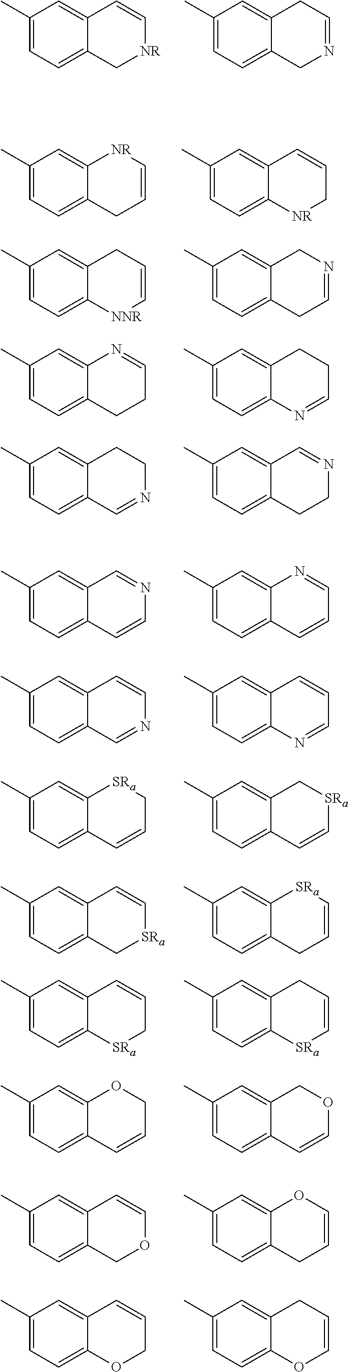

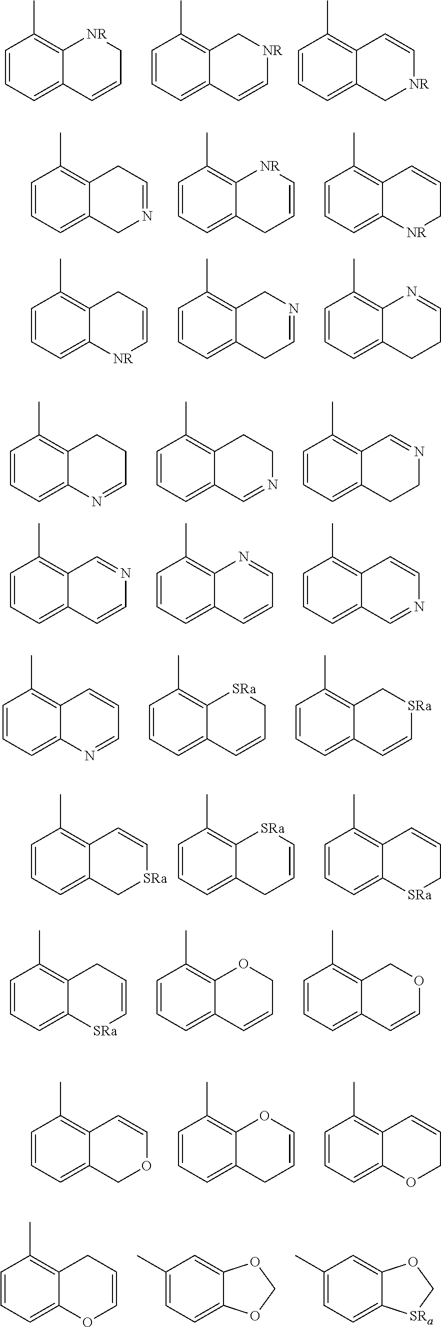

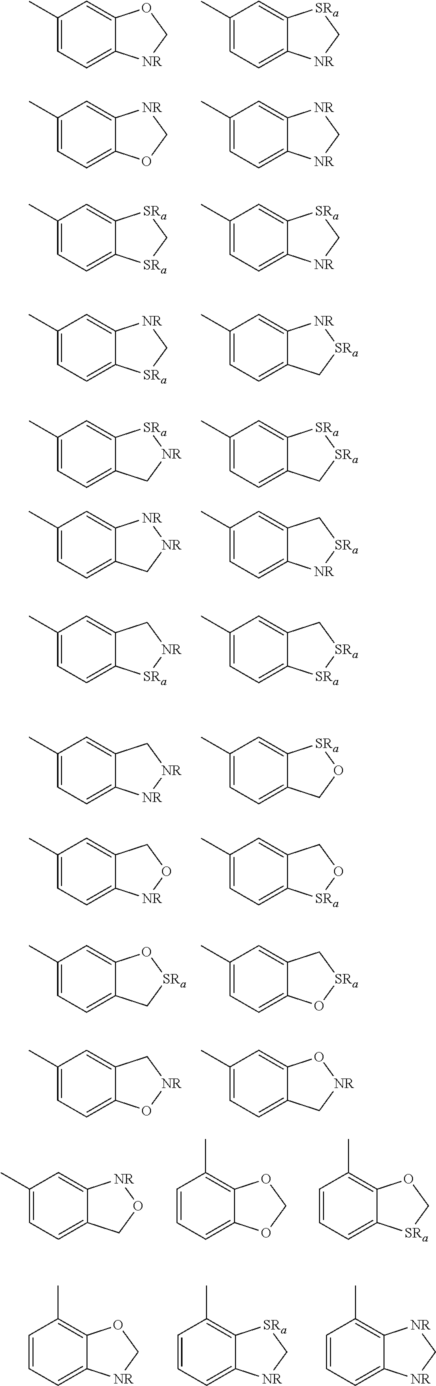

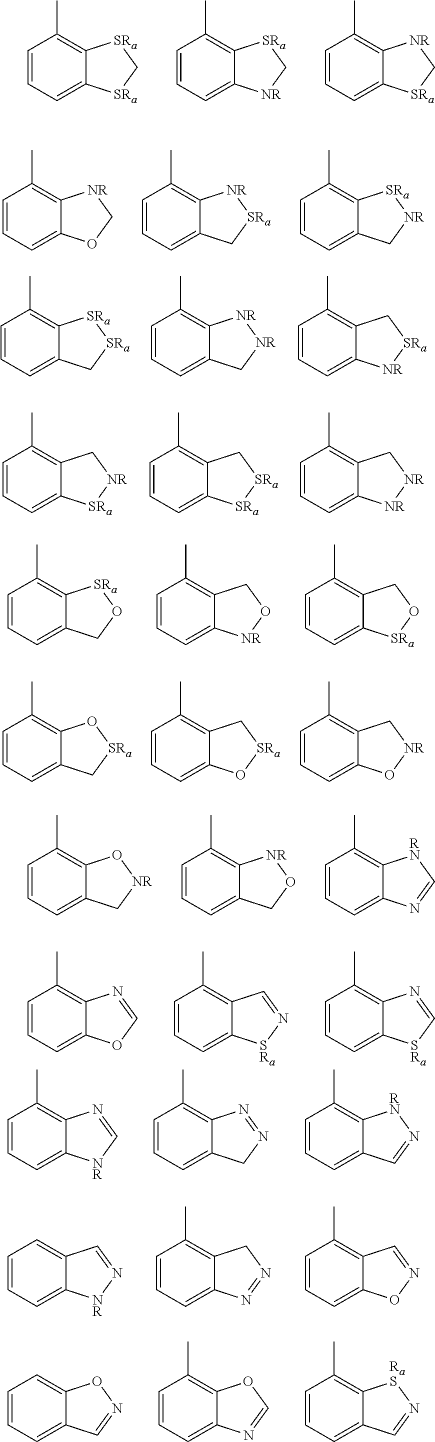

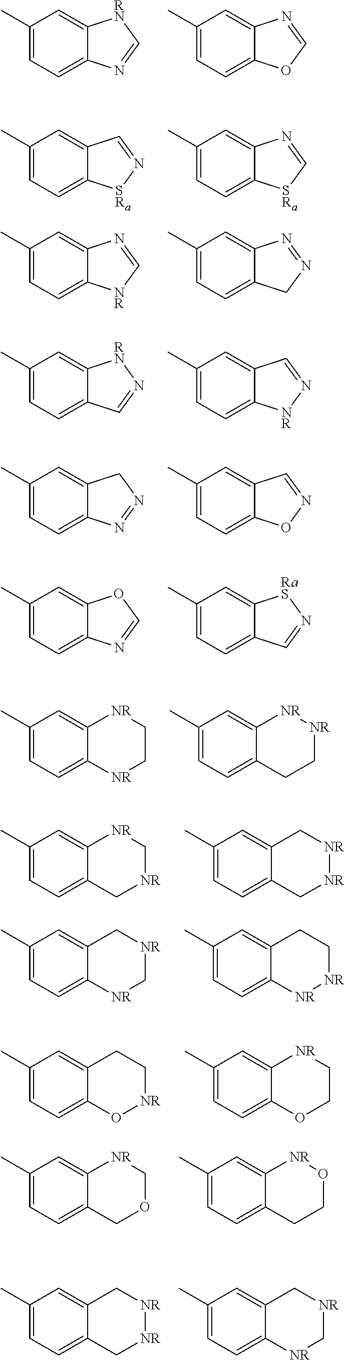

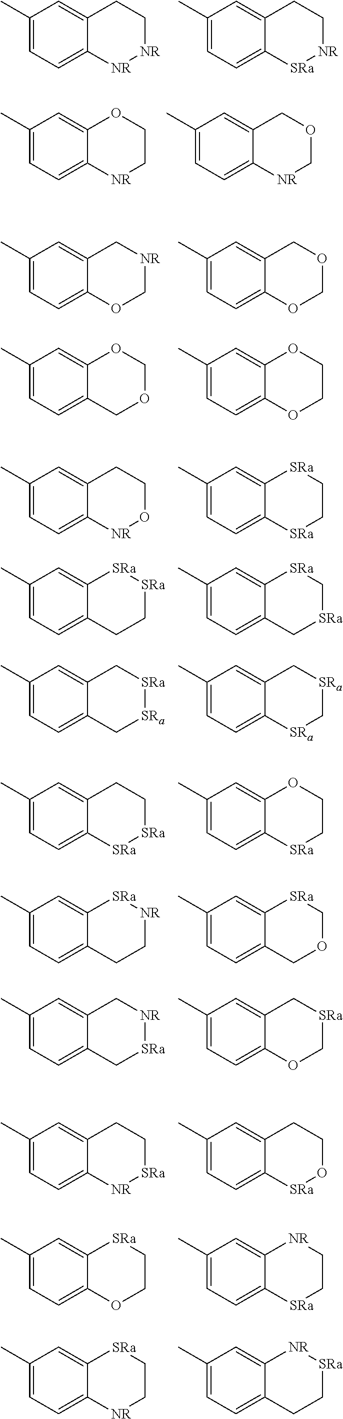

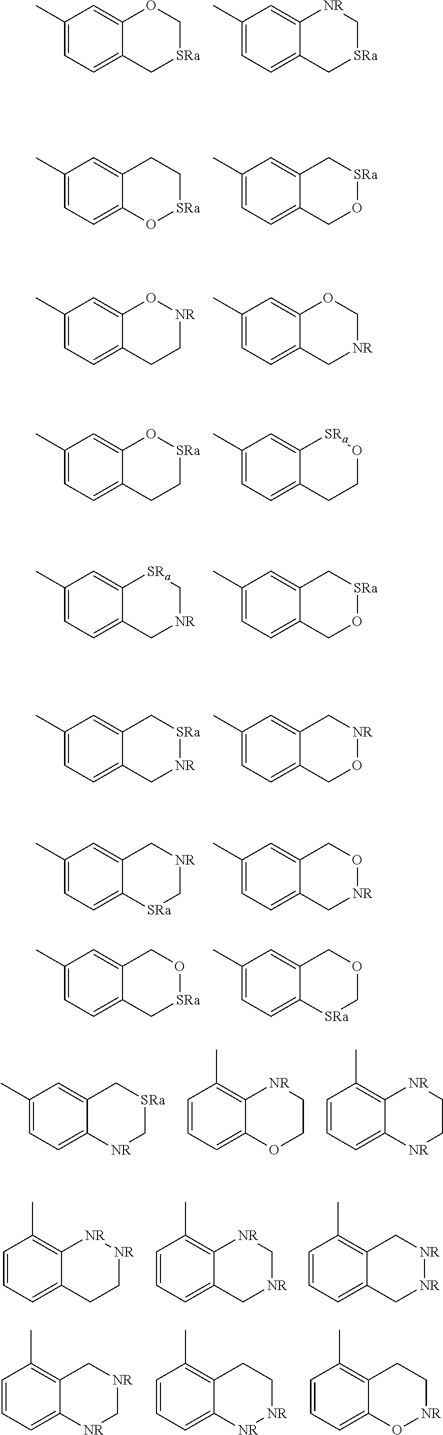

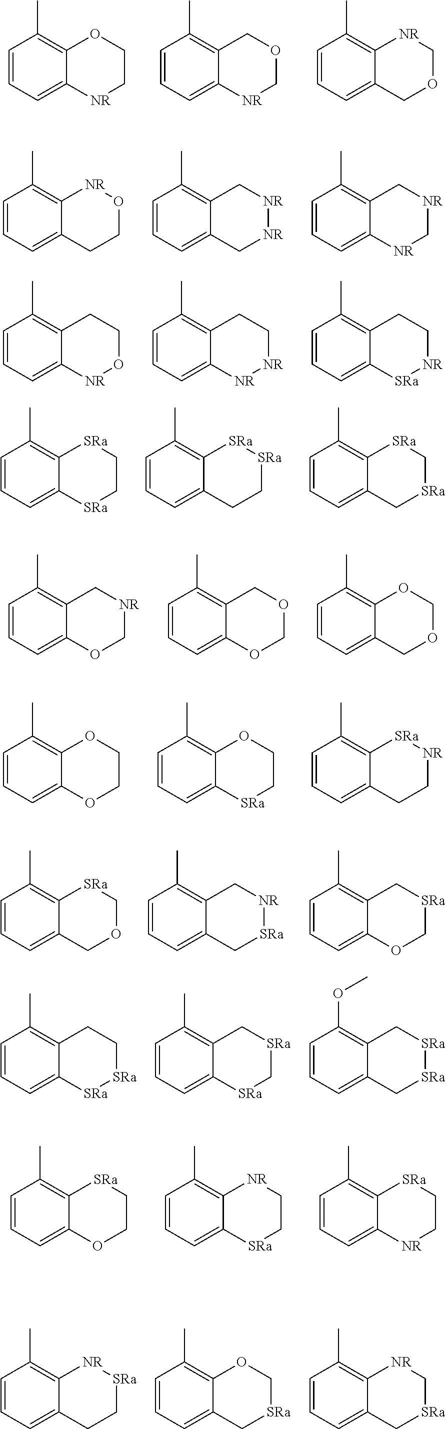

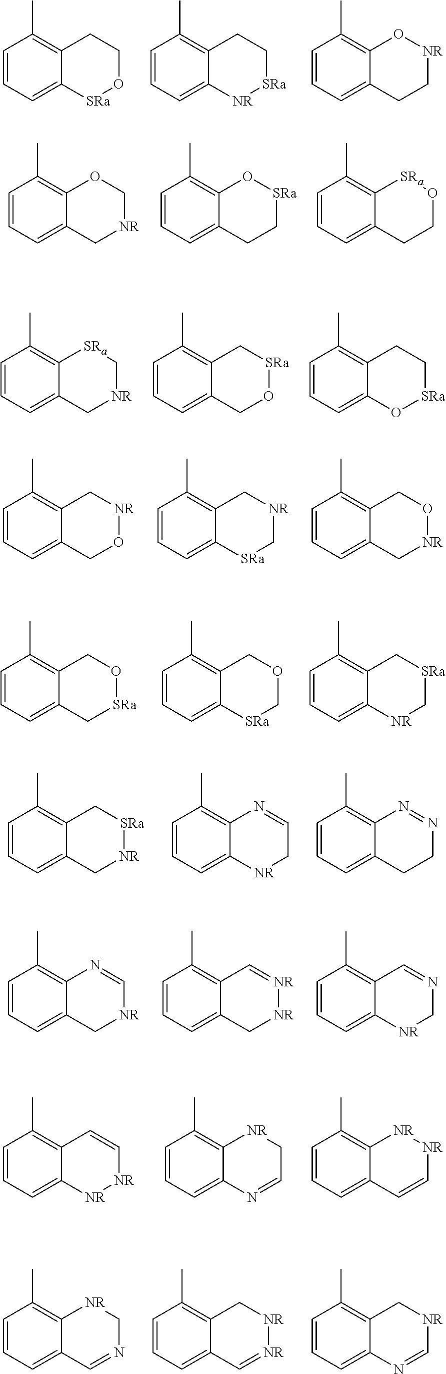

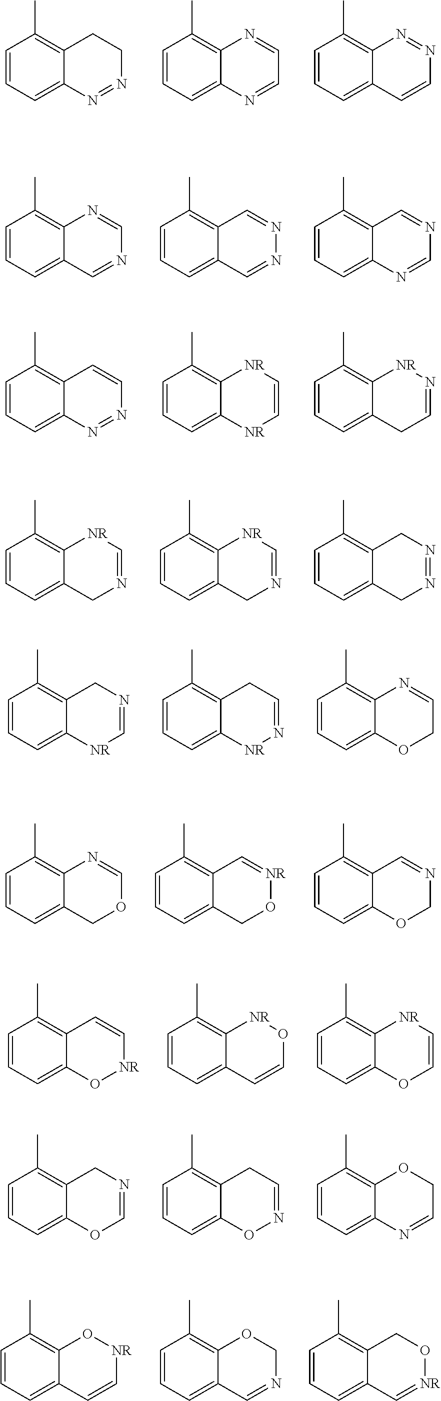

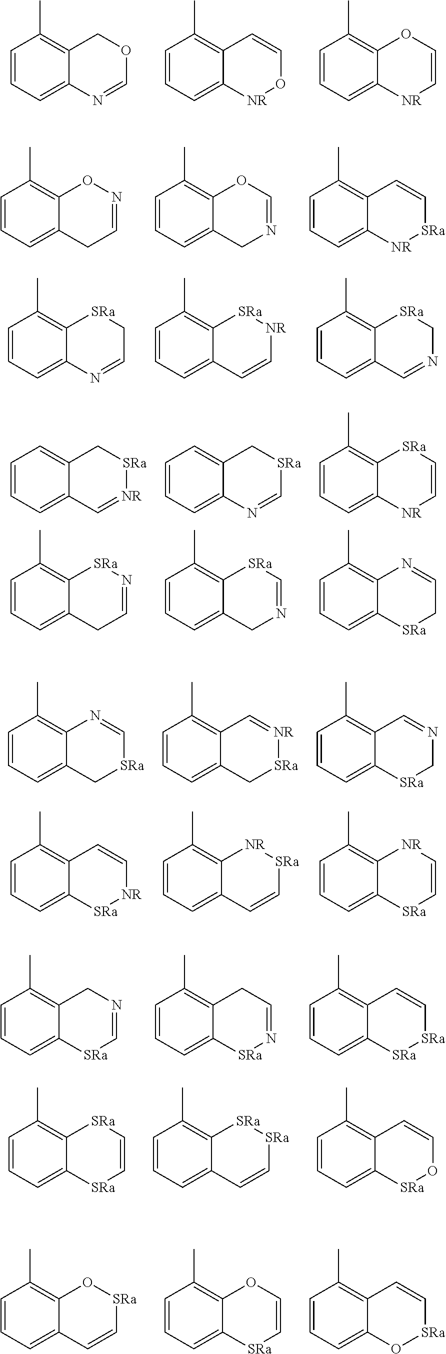

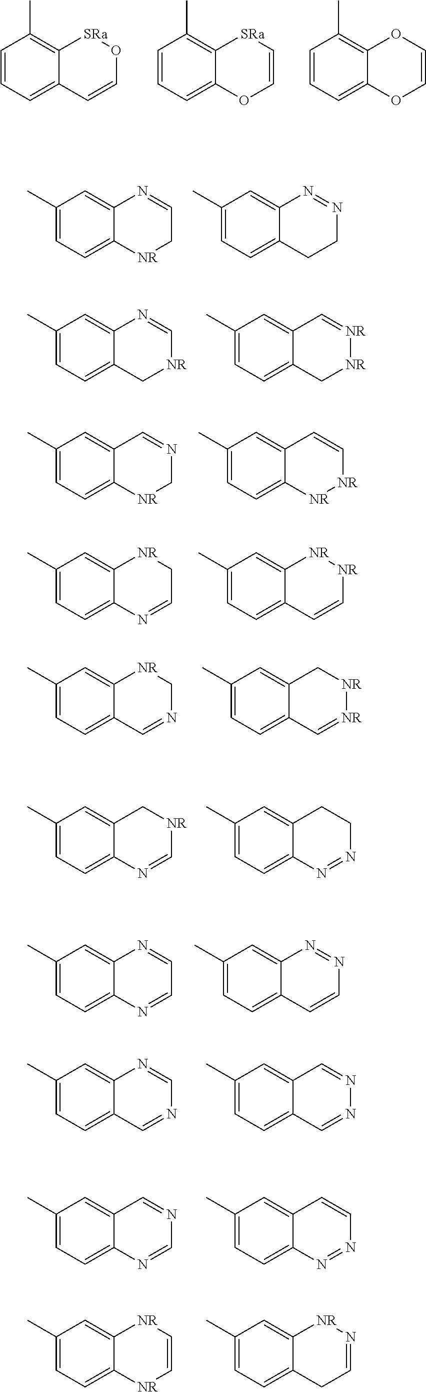

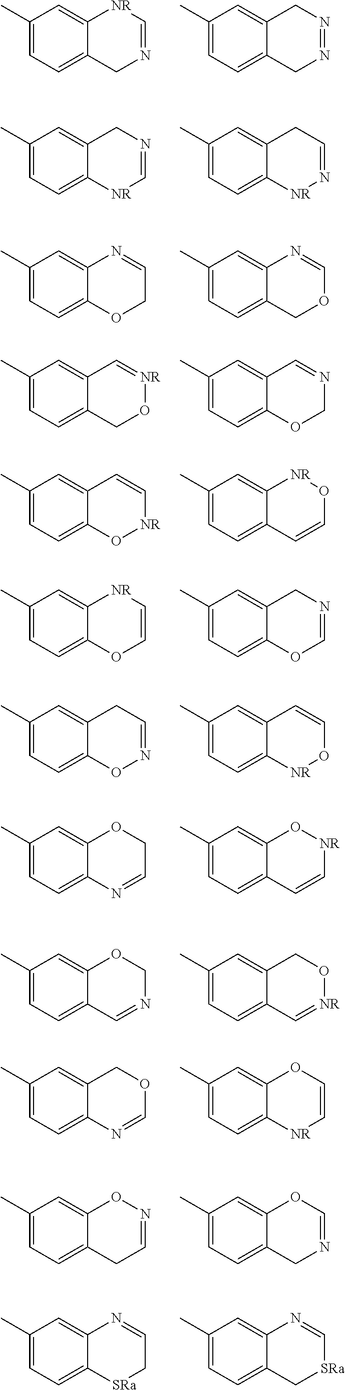

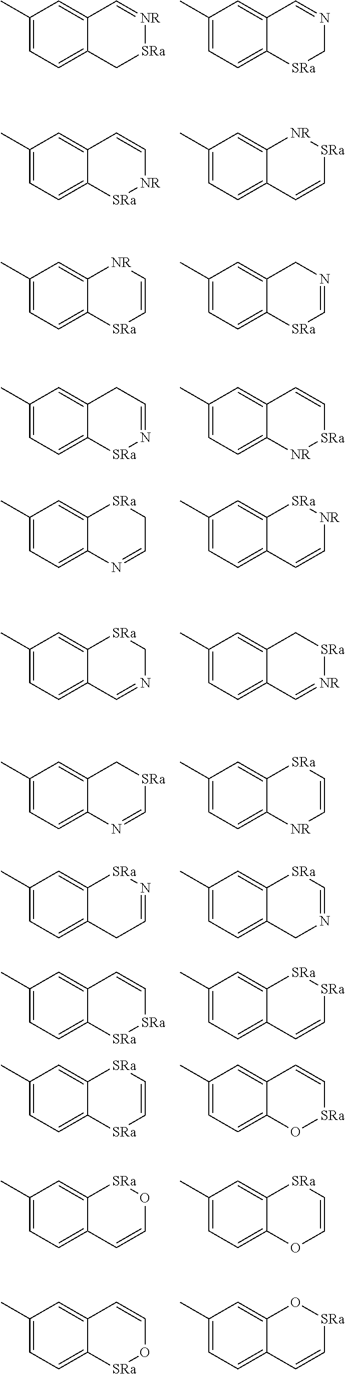

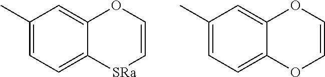

wherein R and Rₐ are selected from the group consisting of hydrogen, C₁₋₆alkyl, C₃₋₈cycloalkyl, heterocycloalkyl, aryl, and heteroaryl.

What is claimed:

1. A compound having a structural formula 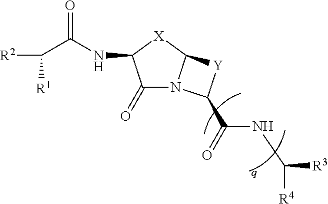
wherein X is (CH₂)ₙ and n is 1-6, wherein one CH₂ can be substituted by a heteroatom and CH₂ optionally can be substituted;
Y is (CH₂)ₘ and m is 1-3, wherein one CH₂ can be substituted by a heteroatom and CH₂ can be substituted;
q is 0 or 1;
R¹ is 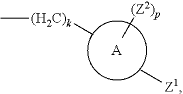
A is phenyl or a 5 or 6-membered heteroaryl ring, k is 0, 1, 2, and p is 0 or 1, or R¹ is (CH₂)₁₋₆P(O)(OR^{a})₂;
Z¹, Z², independently, are OPO(OR^{a})₂, CH₂PO₃(R^{a})₂, OCH₂PO₃(H)(R^{a}), OCHFPO₃(R^{a})₂, (CH₂)₁₋₆CO₂R^{a}, (CH₂)₁₋₆P(O)(OH)(R^{a}), OCF₂PO₃(R^{a})₂, OCH(COOR^{a})₂, O(CH₂)₁₋₃CH(COOR)₂, O(CH₂)₁₋₃COOR^{a}, O(CH₂)₁₋₃COR^{a}, OR^{a}, CON(R^{a})₂, or COOR^{a};
R² is H, NR^{a}R^{b}, NR^{a}C(═O)R^{b}, NR^{a}SOR^{b}, NR^{a}SO₂R^{b}, NR^{a}C(═O)OR^{b}, NR^{a}C(═O)NR^{b}R^{c}, NR^{a}C(═S)NR^{b}R^{c}, or NR^{a}C(═NH)NR^{b}R^{c};
or R² is null and R¹ is 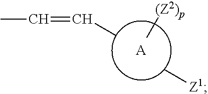
or R¹ and R² are taken together with the carbon atom to which they are attached to fowl a 5- to 10-membered monocyclic or bicyclic heteroaryl group substituted having a Z¹ group;
R³ is (CH₂)ⱼC(═O)NR^{a}R^{b}, (CH₂)ⱼNR^{a}C(═O)R^{b}, (CH₂)ⱼC(═O)R^{a}, (CH₂)ⱼNR^{a}(C═O)NR^{b}R^{c}, (CH₂)ⱼCH(OH)CH₂OR^{a}, C₁₋₆alkyl, NR^{a}C(═O)OR^{b}, NR^{a}R^{b}, (CH₂)ⱼNR^{a}(═NH)R^{b}R^{c}, C₁₋₆alkylNR^{a}R^{b}, (CH₂)ⱼNR^{a}C(═S)NR^{b}R^{c}, (CH₂)ⱼNR^{a}NR^{b},R³ is (CH₂)ⱼC(═O)NR^{a}R^{b}, (CH₂)ⱼNR^{a}C(═O)R^{b}, (CH₂)ⱼC(═O)R^{a}, (CH₂)ⱼNR^{a}C(═O)NR^{b}R^{c}, (CH₂)ⱼCH(OH)CH₂OR^{a}, C₁₋₆alkyl, NR^{a}C(═O)OR^{b}, NR^{a}R^{b}, C₁₋₆alkylNR^{a}R^{b}, (CH₂)ⱼNR^{a}C(═S)NR^{b}R^{c}, (CH₂)ⱼNR^{a}R^{b}, 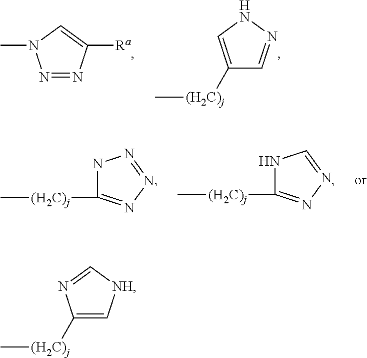
and j is 1, 2, 3, or 4;
R⁴ is H, R^{a}, or CONR^{a}R^{b}; and
R^{a}, R^{b}, R^{e}, independently, is hydrogen, alkyl, substituted alkyl, aryl, substituted aryl, heteroaryl, substituted heteroaryl, C₃₋₈cycloalkyl, heterocycloalkyl, C₁₋₆alkyleneheterocycloalkyl, substituted C₁₋₆alkyleneheterocycloalkyl, C₁₋₆alkylenearyl, substituted C₁₋₆alkylenearyl, C₁₋₆alkyleneheteroaryl, substituted C₁₋₆alkyleneheteroaryl, and (CH₂)₁₋₃(OCH₂)₁₋₃(OCH₂CH₂)₁₋₆NHR^{d}; and
R^{d} is hydrogen or 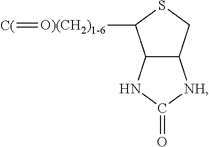
or a pharmaceutically acceptable salt, prodrug, hydrate, or solvate thereof.

2. The compound of claim 1 having a structure 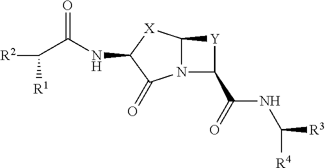
wherein X is (CH₂)ₙ and n is 1-6, wherein one CH₂ can be substituted by a O, S, or NR^{a}, and CH₂ optionally can be substituted;
Y is (CH₂)ₘ and m is 1-3, wherein one CH₂ can be substituted by a O, S, or NR^{a}, and CH₂ can be substituted;
R¹ is 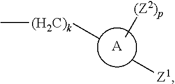
A is phenyl or a 5 or 6-membered heteroaryl ring, k is 0, 1, 2, and p is 0 or 1, or R¹ is (CH₂)₁₋₆P(O)(OR^{a})₂;
Z¹, Z², independently, are OPO(OR^{a})₂, CH₂PO₃(R^{a})₂, OCH₂PO₃(H)(R^{a}), OCHFPO₃(R^{a})₂, (CH₂)₁₋₆CO₂R^{a}, (CH₂)₁₋₆P(O)(OH)(R^{a}), OCF₂PO₃(R^{a})₂, OCH(COOR^{a})₂, O(CH₂)₁₋₃CH(COOR^{a})₂, O(CH₂)₁₋₃COOR^{a}, O(CH₂)₁₋₃COR^{a}, OR^{a}, CON(R^{a})₂, or COOR^{a};
R² is H, NR^{a}R^{b}, NR^{a}C(═O)R^{b}, NR^{a}SOR^{b}, NR^{a}SO₂R^{b}, NR^{a}C(═O)OR^{b}, NR^{a}C(═O)NR^{b}R^{c}, NR^{a}C(═S)NR^{b}R^{c}, or NR^{a}C(═NH)NR^{b}R^{c};
or R² is null and R is 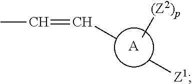
or R¹ and R² are taken together with the carbon atom to which they are attached to form a 5- to 10-membered monocyclic or bicyclic heteroaryl group substituted having a Z¹ group;
R³ is (CH₂)ⱼC(═O)NR^{a}R^{b}, (CH₂)ⱼNR^{a}C(═O)R^{b}, (CH₂)ⱼC(═O)R^{a}, (CH₂)ⱼNR^{a}C(═O)NR^{b}R^{c}, (CH₂)ⱼCH(OH)CH₂OR^{a}, C₁₋₆alkyl, NR^{a}C(═O)OR^{b}, NR^{a}R^{b}, (CH₂)ⱼNR^{a}(═NH)R^{b}R^{c}, C₁₋₆alkylNR^{a}R^{b}, (CH₂)ⱼNR^{a}C(═S)NR^{b}R^{c}, (CH₂)NR^{a}R^{b}, (CH₂)ⱼNR^{a}C(═S)NR^{b}R^{c}, (CH₂)ⱼNR^{a}R^{b}, 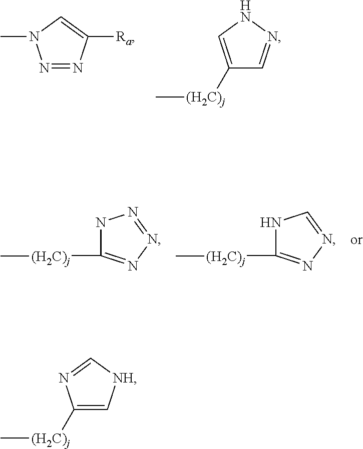
and j is 1, 2, 3, or 4;
R⁴ is H, R^{a}, or CONR^{a}R^{b}; and
R^{a}, R^{b}, R^{c}, independently, is hydrogen, alkyl, substituted alkyl, aryl, substituted aryl, heteroaryl, substituted heteroaryl, C₃₋₈cycloalkyl, heterocycloalkyl, C₁₋₆olkyleneheterocycloalkyl, substituted C₁₋₆alkyleneheterocycloalkyl, C₁₋₆alkylenearyl, substituted C₁₋₆alkylenearyl, C₁₋₆alkyleneheteroaryl, substituted C₁₋₆alkyleneheteroaryl, and (CH₂)₁₋₃(OCH₂)₁₋₃(OCH₂CH₂)₁₋₆NHR^{d}; and
R^{d} is hydrogen or 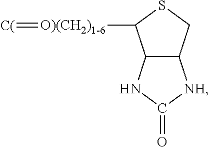
or a pharmaceutically acceptable salt, prodrug, hydrate, or solvate thereof.

3. The compound of claim 1 wherein one or more CH₂ group of X, Y, or both, independently, is substituted with halo, CF₃, OCF₃, OH, alkoxy, NO₂, CN, alkylamino, or amino.

4. The compound of claim 1 wherein the A ring is selected from the group consisting of phenyl, furanyl, thienyl, pyrrolyl, oxazolyl, thiazolyl, imidazolyl, pyrazolyl, isoxazolyl, isothiazolyl, 1,2,3,-oxadiazolyl, 1,2,3,-triazolyl, 1,3,4-thiadiazolyl, 1,2,4-oxadiazolyl, 1,2,5-oxadiazolyl, 1,3,4-oxadiazolyl, 1,2,3,4-oxatriazolyl, 1,2,3,5-oxatriazolyl, pyridinyl, pyridazinyl, pyrazinyl, and 1,3,5-triazinyl.

5. The compound of claim 1 wherein the A ring is phenyl.

6. The compound of claim 1 wherein the k is 1 or 2.

7. The compound of claim 1 wherein the Z¹ is selected from the group consisting of OPO₃(R^{a})₂, OCH(CO₂R^{a})₂, (CH₂)₂CO₂R^{a}, OR^{a}, OCH₂CO₂R^{a}, and (CH₂)₁₋₄PO₃(R^{a})₂.

8. The compound of claim 7 wherein the Z¹ is selected from the group consisting of OPO₃H₂, OCH(CO₂H)₂, (CH₂)₂CO₂(tBu), (CH₂)₂CO₂H, OH, OCH₂CO₂C₂H₅, OCH(CO₂C₂H₅)₂, OCH₂CO₂H, OPO(OCH₃)₂, CH₃PO₃H₂, CH₂P(O)(OH)(CH₃), (CH₂)₄P(O)(OH)(CH₃), OCH₂PO₃H₂, and OCH₂PO₃(H)(C₄H₁₀).

9. The compound of claim 1 wherein the Z² is CO₂R^{a}.

10. The compound of claim 1 wherein R² is selected from the group consisting of H, N(R^{a})₂, and NR^{a}C(═O)R^{b}.

11. The compound of claim 10 wherein R² is selected from the group consisting of H, NH₂, NHC(═O)CH₃, N(CH₃)₂, NHCH₃, NHC(═O)(CH₂)₁₄CH₃, N[(CH₂)₇CH₃]₂,

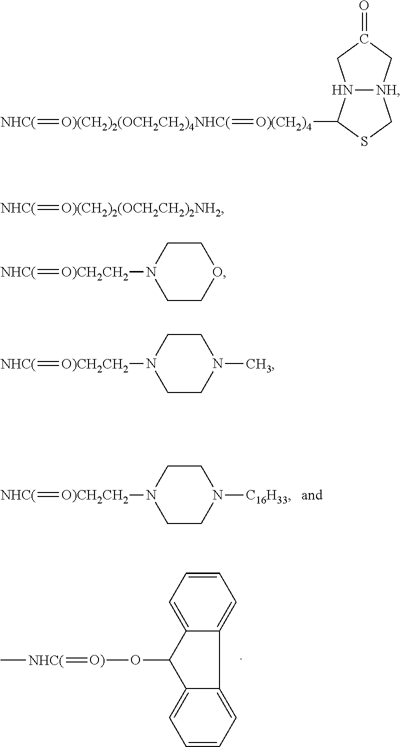

12. The compound of claim 1 wherein R³ is selected from the group consisting of (CH₂)ⱼC(═O)NR^{a}R^{b}, C₁₋₆alkyl, NR^{a}R^{b}, (CH₂)ⱼCH(OH)CH₂OR^{a}, NR^{a}C(═O)OR^{b}, 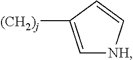
(CH₂)ⱼNR^{a}R^{b}, (CH₂)ⱼNR^{a}C(═O)R^{b}, (CH₂)ⱼNR^{a}(═NH)NR^{b}R^{c}, and

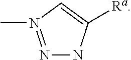

13. The compound of claim 12 wherein R³ is selected from the group consisting of (CH₂)₂C(═O)NH₂, CH₂CH(OH)CH₂OH, CH₃, CH₃CH₂, NH₂, NHC(═O)OCH₂C₆H₅, (CH₂)₃NH₂, (CH₂)₃N(CH₃)₂, (CH₂)₃NHC(═O)CH₃, (CH₂)₁₋₃NH(═NH)NH, (CH₂)₃NH₂, (CH₂)₂C(═O)NH(CH₃), (CH₂)₂C(═O)N(CH₃)₂,

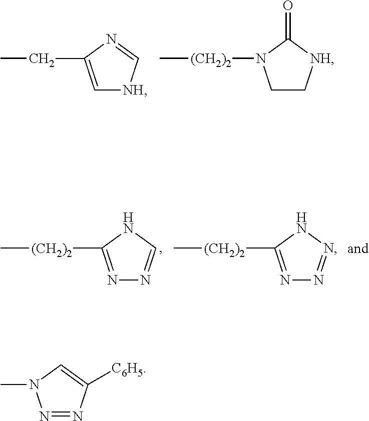

14. The compound of claim 1 wherein R⁴ is selected from the group consisting of H or C(═O)NR^{a}R^{b}.

15. The compound of claim 14 wherein R⁴ is selected from the group consisting of H, C(═O)NHCH₂C₆H₅, C(═O)NHCH₂CH₂C₆H₅, C(═O)NH(CH₂)₄C₆H₅, C(═O)NH(CH₂)₆C₆H₅, and C(═O)NHCH₃.

16. The compound of claim 1 wherein or R² is null and R¹ is

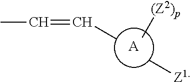

17. The compound of claim 1 wherein R¹ and R² are taken together with the carbon atom they are attached to form a 5- to 10-member monocyclic or bicyclic heteroaryl group substituted having a Z¹ group.

18. The compound of claim 1 wherein the heteroaryl group is selected from the group consisting of

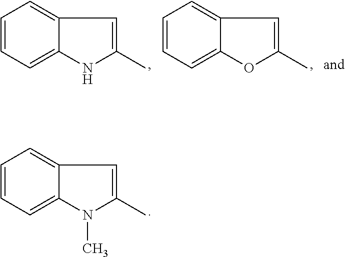

19. The compound of claim 1 having a structure 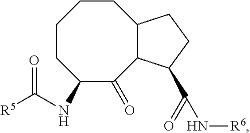
wherein
R⁵ is 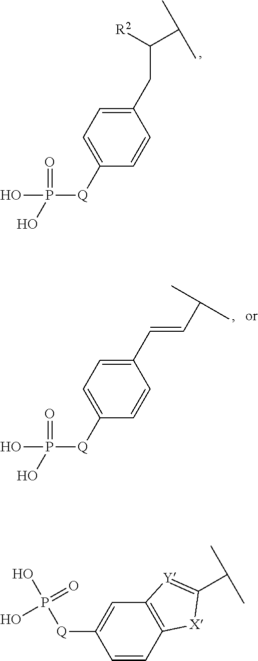
Q is O, CH₂, OCH₂, CF₂, CFH;
X′ is O, NH;
Y′ is CH, N, O; and
R⁶ is

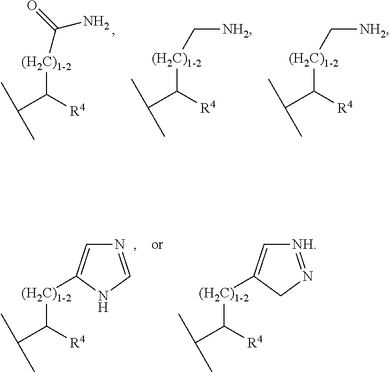

20. A compound selected from the group consisting of the listing of compounds in Table 1 and Examples 1-54 above.

21. A compound selected from the group consisting of the listing of compounds in Table 2 and Examples 55-71 above.

22. A method of treating a disease or condition wherein inhibition of STAT3 provides a benefit comprising administering a therapeutically effective amount of a compound of claim 1 to an individual in need thereof.

23. The method of claim 22 further comprising administering a therapeutically effective amount of a second therapeutic agent useful in the treatment of the disease or condition.

24. The method of claim 23 wherein the compound of claim 1 and the second therapeutic agent are administered simultaneously.

25. The method of claim 23 wherein the compound of claim 1 and the second therapeutic agent are administered separately.

26. The method of claim 22 wherein the disease or condition is a cancer.

27. The method of claim 26 further comprising administering a therapeutically effective amount of one or more of a chemotherapeutic agent and radiation.

28. A composition comprising (a) compound of claim 1, (b) an optional second therapeutic agent useful in the treatment of a disease or condition wherein inhibition of STAT3 provides a benefit, and (c) an excipient and/or pharmaceutically acceptable carrier.

29. The composition of claim 28 comprising a second therapeutic agent.

30. The composition of claim 29 wherein the second therapeutic agent comprises a chemotherapeutic agent useful in the treatment of a cancer.
